SEQUENCE LISTING (28 sequences)

SEQ ID NO: 1 - DNA, 21 bp, artificial sequence
  shRNA
cccatcatactttaatgtgta

SEQ ID NO: 2 - DNA, 21 bp, artificial sequence
  shRNA sequence
gccctggaactgtgtttagaa

SEQ ID NO: 3 - DNA, 21 bp, artificial sequence
  shRNA sequence
cggtaaattcattcgtatcaa

SEQ ID NO: 4 - DNA, 21 bp, artificial sequence
  shRNA sequence
gcacacattgacacagccaat

SEQ ID NO: 5 - DNA, 21 bp, artificial sequence
  shRNA sequence
gccatacaacaaataccgctt

SEQ ID NO: 6 - DNA, 21 bp, artificial sequence
  shRNA sequence
gcgatactactcagggcttat

SEQ ID NO: 7 - DNA, 21 bp, artificial sequence
  shRNA sequence
caacaagatgaagagcaccaa

SEQ ID NO: 8 - DNA, 19 bp, artificial sequence
  primer
gtggccttgtcgctgtctt

SEQ ID NO: 9 - DNA, 23 bp, artificial sequence
  primer
gcgcttggagtgatagaaatctg

SEQ ID NO: 10 - DNA, 21 bp, artificial sequence
  primer
ctgcgatgaagagcatggttt

SEQ ID NO: 11 - DNA, 20 bp, artificial sequence
  primer
ccataggcgatttctgggac

SEQ ID NO: 12 - DNA, 21 bp, artificial sequence
  primer
atgcgtccaccaagaagctga

SEQ ID NO: 13 - DNA, 22 bp, artificial sequence
  primer
agcaatcatcctctgcagctcc

SEQ ID NO: 14 - DNA, 24 bp, artificial sequence
  primer
ttcggccttctcctcgctgtcgtc

SEQ ID NO: 15 - DNA, 24 bp, artificial sequence
  primer
tggcgtaagtgagcattctggtga

SEQ ID NO: 16 - DNA, 19 bp, artificial sequence
  primer
tgtgtgcgacactgtgctc

SEQ ID NO: 17 - DNA, 21 bp, artificial sequence
  primer
tcggctaggtagcggtagtag

SEQ ID NO: 18 - DNA, 23 bp, artificial sequence
  primer
gatcagtcaacgggggacataaa

SEQ ID NO: 19 - DNA, 23 bp, artificial sequence
  primer
cttgcgctcatcttaggctttgt

SEQ ID NO: 20 - DNA, 21 bp, artificial sequence
  primer
gtgagcgcttcccagatgaga

SEQ ID NO: 21 - DNA, 20 bp, artificial sequence
  primer
tgccggagtcgacaatgatg

SEQ ID NO: 22 - protein, 448 aa, human
IKVGRDYVQKAQTKEQADFAIEALAKATYERMFRWLVLRINKALDKTKRQGASFIGILDIAGFEIFDLNSFEQLCINYTNEKLQQLFNHTMFILEQEEYQREGIEWNFIDFGLDLQPCIDLIEKPAGPPGILALLDEECWFPKATDKSFVEKVMQEQGTHPKFQKPKQLKDKADFCIIHYAGKVDYKADEWLMKNMDPLNDNIATLLHQSSDKFVSELWKDVDRIIGLDQVAGMSETALPGAFKTRKGMFRTVGQLYKEQLAKLMATLRNTNPNFVRCIIPNHEKKAGKLDPHLVLDQLRCNGVLEGIRICRQGFPNRVVFQEFRQRYEILTPNSIPKGFMDGKQACVLMIKALELDSNLYRIGQSKVFFRAGVLAHLEEERDLKITDVIIGFQACCRGYLARKAFAKRQQQLTAMKVLQRNCAAYLKLRNWQWWRLFTKVKPLLQVS

SEQ ID NO: 23 - protein, 448 aa, canine
IKVGRDYVQKAQTKEQADFAIEALAKATYERMFRWLVLRINKALDKTKRQGASFIGILDIAGFEIFDLNSFEQLCINYTNEKLQQLFNHTMFILEQEEYQREGIEWNFIDFGLDLQPCIDLIEKPAGPPGILALLDEECWFPKATDKSFVEKVVQEQGTHPKFQKPKQLKDKADFCIIHYAGKVDYKADEWLMKNMDPLNDNIATLLHQSSDKFVSELWKDVDRIIGLDQVAGMSETALPGAFKTRKGMFRTVGQLYKEQLAKLMATLRNTNPNFVRCIIPNHEKKAGKLDPHLVLDQLRCNGVLEGIRICRQGFPNRVVFQEFRQRYEILTPNSIPKGFMDGKQACVLMIKALELDSNLYRIGQSKVFFRAGVLAHLEEERDLKITDVIIGFQACCRGYLARKAFAKRQQQLTAMKVLQRNCAAYLKLRNWQWWRLFTKVKPLLQVS

SEQ ID NO: 24 - protein, 448 aa, mouse
IKVGRDYVQKAQTKEQADFAIEALAKATYERMFRWLVLRINKALDKTKRQGASFIGILDIAGFEIFDLNSFEQLCINYTNEKLQQLFNHTMFILEQEEYQREGIEWNFIDFGLDLQPCIDLIEKPAGPPGILALLDEECWFPKATDKSFVEKVVQEQGTHPKFQKPKQLKDKADFCIIHYAGKVDYKADEWLMKNMDPLNDNIATLLHQSSDKFVSELWKDVDRIIGLDQVAGMSETALPGAFKTRKGMFRTVGQLYKEQLAKLMATLRNTNPNFVRCIIPNHEKKAGKLDPHLVLDQLRCNGVLEGIRICRQGFPNRVVFQEFRQRYEILTPNSIPKGFMDGKQACVLMIKALELDSNLYRIGQSKVFFRAGVLAHLEEERDLKITDVIIGFQACCRGYLARKAFAKRQQQLTAMKVLQRNCAAYLRLRNWQWWRLFTKVKPLLNSI

SEQ ID NO: 25 - protein, 448 aa, rat
IKVGRDYVQKAQTKEQADFAIEALAKATYERMFRWLVLRINKALDKTKRQGASFIGILDIAGFEIFDLNSFEQLCINYTNEKLQQLFNHTMFILEQEEYQREGIEWNFIDFGLDLQPCIDLIEKPAGPPGILALLDEECWFPKATDKSFVEKVVQEQGTHPKFQKPKQLKDKADFCIIHYAGKVDYKADEWLMKNMDPLNDNIATLLHQSSDKFVSELWKDVDRIIGLDQVAGMSETALPGAFKTRKGMFRTVGQLYKEQLAKLMATLRNTNPNFVCCIIPNHEKKAGKLDPHLVLDQLRCNGVLEGIRICRQGFPNRVVFQEFRQRYEILTPNSIPKGFMDGKQACVLMIKALELDSNLYRIGQSKVFFRSGVLAHLEEERDLKITDVIIGFQACCRGYLARKAFAKRQQQLTAMKVLQRNCAAYLRLRNWQWWRLFTKVKPLLNSI

SEQ ID NO: 26 - protein, 448 aa, chicken
IKVGRDYVQKAQTKEQADFAIEALAKATYEQMFRWLVMRINKALDKTKRQGASFIGILDIAGFEIFELNSFEQLCINYTNEKLQQLFNHTMFILEQEEYQNEGIEWNFIDFGLDLQPCIDLIEKPAGPPGILALLDEECWFPKATDKSFVEKVVQEQGTHPKFQKPKQLKDKADFCIIHYAGKVDYKADEWLMKNMDPLNDNIATLLHQSSDKFVSELWKDVDRIVGLDQVAGMSETALPGAFKTRKGMFRTVGQLYKEQLAKLMATLRNTNPNFVRCIIPNHEKKAGKLDPHLVLDQLRCNGVLEGIRICRQGFPNRVVFQEFRQRYEILTPNAIPKGFMDGKQACVLMIKALELDSNLYRIGQSKVFFRAGVLAHLEEERDLKITDVIIGFQACCRGYLARKAFAKRQQQLTAMKVLQRNCAAYLKLRNWQWWRLFTKVKPLLQVS

SEQ ID NO: 27 - protein, 427 aa, Dyctyostelium discoideum
ILAGRDLVAQHLNVEKSSSSRDALVKALYGRLFLWLVKKINNVLCQERKAYFIGVLDISGFEIFKVNSFEQLCINYTNEKLQQFFNHHMFKLEQEEYLKEKINWTFIDFGLDSQATIDLIDGRQPPGILALLDEQSVFPNATDNTLITKLHSHFSKKNAKYEEPRFSKTEFGVTHYAGQVMYEIQDWLEKNKDPLQQDLELCFKDSSDNVVTKLFNDPNIASRAKKGANFITVAAQYKEQLASLMATLETTNPHFVRCIIPNNKQLPAKLEDKVVLDQLRCNGVLEGIRITRKGFPNRIIYADFVKRYYLLAPNVPRDAEDSQKATDAVLKHLNIDPEQYRFGITKIFFRAGQLARIEEAREQRISEIIKAIQAATRGWIARKVYKQAREHTVAARIIQQNLRAYIDFKSWPWWKLFSKARPLLKRR

SEQ ID NO: 28 - protein, 1960 aa, human
MAQQAADKYLYVDKNFINNPLAQADWAAKKLVWVPSDKSGFEPASLKEEVGEEAIVELVENGKKVKVNKDDIQKMNPPKFSKVEDMAELTCLNEASVLHNLKERYYSGLIYTYSGLFCVVINPYKNLPIYSEEIVEMYKGKKRHEMPPHIYAITDTAYRSMMQDREDQSILCTGESGAGKTENTKKVIQYLAYVASSHKSKKDQGELERQLLQANPILEAFGNAKTVKNDNSSRFGKFIRINFDVNGYIVGANIETYLLEKSRAIRQAKEERTFHIFYYLLSGAGEHLKTDLLLEPYNKYRFLSNGHVTIPGQQDKDMFQETMEAMRIMGIPEEEQMGLLRVISGVLQLGNIVFKKERNTDQASMPDNTAAQKVSHLLGINVTDFTRGILTPRIKVGRDYVQKAQTKEQADFAIEALAKATYERMFRWLVLRINKALDKTKRQGASFIGILDIAGFEIFDLNSFEQLCINYTNEKLQQLFNHTMFILEQEEYQREGIEWNFIDFGLDLQPCIDLIEKPAGPPGILALLDEECWFPKATDKSFVEKVMQEQGTHPKFQKPKQLKDKADFCIIHYAGKVDYKADEWLMKNMDPLNDNIATLLHQSSDKFVSELWKDVDRIIGLDQVAGMSETALPGAFKTRKGMFRTVGQLYKEQLAKLMATLRNTNPNFVRCIIPNHEKKAGKLDPHLVLDQLRCNGVLEGIRICRQGFPNRVVFQEFRQRYEILTPNSIPKGFMDGKQACVLMIKALELDSNLYRIGQSKVFFRAGVLAHLEEERDLKITDVIIGFQACCRGYLARKAFAKRQQQLTAMKVLQRNCAAYLKLRNWQWWRLFTKVKPLLQVSRQEEEMMAKEEELVKVREKQLAAENRLTEMETLQSQLMAEKLQLQEQLQAETELCAEAEELRARLTAKKQELEEICHDLEARVEEEEERCQHLQAEKKKMQQNIQELEEQLEEEESARQKLQLEKVTTEAKLKKLEEEQIILEDQNCKLAKEKKLLEDRIAEFTTNLTEEEEKSKSLAKLKNKHEAMITDLEERLRREEKQRQELEKTRRKLEGDSTDLSDQIAELQAQIAELKMQLAKKEEELQAALARVEEEAAQKNMALKKIRELESQISELQEDLESERASRNKAEKQKRDLGEELEALKTELEDTLDSTAAQQELRSKREQEVNILKKTLEEEAKTHEAQIQEMRQKHSQAVEELAEQLEQTKRVKANLEKAKQTLENERGELANEVKVLLQGKGDSEHKRKKVEAQLQELQVKFNEGERVRTELADKVTKLQVELDNVTGLLSQSDSKSSKLTKDFSALESQLQDTQELLQEENRQKLSLSTKLKQVEDEKNSFREQLEEEEEAKHNLEKQIATLHAQVADMKKKMEDSVGCLETAEEVKRKLQKDLEGLSQRHEEKVAAYDKLEKTKTRLQQELDDLLVDLDHQRQSACNLEKKQKKFDQLLAEEKTISAKYAEERDRAEAEAREKETKALSLARALEEAMEQKAELERLNKQFRTEMEDLMSSKDDVGKSVHELEKSKRALEQQVEEMKTQLEELEDELQATEDAKLRLEVNLQAMKAQFERDLQGRDEQSEEKKKQLVRQVREMEAELEDERKQRSMAVAARKKLEMDLKDLEAHIDSANKNRDEAIKQLRKLQAQMKDCMRELDDTRASREEILAQAKENEKKLKSMEAEMIQLQEELAAAERAKRQAQQERDELADEIANSSGKGALALEEKRRLEARIAQLEEELEEEQGNTELINDRLKKANLQIDQINTDLNLERSHAQKNENARQQLERQNKELKVKLQEMEGTVKSKYKASITALEAKIAQLEEQLDNETKERQAACKQVRRTEKKLKDVLLQVDDERRNAEQYKDQADKASTRLKQLKRQLEEAEEEAQRANASRRKLQRELEDATETADAMNREVSSLKNKLRRGDLPFVVPRRMARKGAGDGSDEEVDGKADGAEAKPAE

(19) United States
(12) Patent Application Publication
(10) Pub. No.: US 2015/0148411 A1
(43) Pub. Date: May 28, 2015

(54) COMPOSITIONS AND METHODS FOR DIAGNOSIS AND THERAPY OF DISORDERS RELATED TO ALTERATIONS OF MYH9

(71) Applicant: THE ROCKEFELLER UNIVERSITY, NEW YORK, NY (US)

(72) Inventors: Daniel Schramek, New York, NY (US); Elaine Fuchs, New York, NY (US)

(21) Appl. No.: 14/553,256

(22) Filed: Nov. 25, 2014

Related U.S. Application Data
(60) Provisional application No. 61/908,498, filed on Nov. 25, 2013.

(51) Int. Cl. C12Q 1/68; G01N 33/574

(52) U.S. Cl. CPC C12Q 1/6886; G01N 33/5748; G01N 33/57415; G01N 33/57407; C12Q 2600/106; C12Q 2600/156; G01N 2333/82; G01N 2333/4712
USPC 514/460; 435/6.14; 435/6.11; 435/7.4; 435/6.12

(57) ABSTRACT

Provided are compositions and methods related to mutations in the Myh9 gene for aiding in diagnosing a subject as having an aggressive form of a cancer, for identifying an individual as a candidate for treatment with a nuclear export inhibitor, for determining whether tumor cells have defective p53 nuclear transportation, and for treating an individual diagnosed with an aggressive cancer.

### CROSS REFERENCE TO RELATED APPLICATIONS

This application claims priority to U.S. provisional application No. 61/908,498, filed on Nov. 25, 2013, the disclosure of which is incorporated herein by reference.

### STATEMENT REGARDING FEDERALLY SPONSORED RESEARCH

This invention was made with government support under Grant No. RD3-AR 27883 awarded by the National Institutes of Health. The government has certain rights in the invention.

### COLOR DRAWINGS

The patent or application file contains at least one drawing executed in color. Copies of this patent or patent application publication with color drawing(s) will be provided by the Office upon request and payment of the necessary fee.

### BACKGROUND

Head and neck squamous cell carcinomas (HNSCCs) are the 6th most common human cancer worldwide, with frequent, often aggressive recurrence and poor prognosis. While there are some established genetic/epigenetic alterations that are positively correlated with HNSCCs, there is an ongoing and unmet need for improved methods of diagnosing and staging HNSCCs, as well as for improved approaches to prophylaxis and therapy of such cancers. The present disclosure is related to these needs.

### SUMMARY OF THE DISCLOSURE

In one embodiment the present disclosure comprises a method diagnosing or aiding in the diagnosis of whether a subject has an aggressive form of a cancer. The method generally comprises testing a sample of a tumor obtained or derived from the subject to determine a mutation in the Myh9 gene (encodes myosin-IIA) or low expression of the Myh9 gene relative to a reference. The low expression can be determined from mRNA and/or protein. In embodiments, 5% or less expression of the Myh9 gene at the mRNA and/or protein level relative to a reference is considered to be low expression. The presence of the mutation and/or the low expression is a diagnosis, or aids in the diagnosis that the individual has an aggressive form of cancer. In embodiments, the mutation is any mutation that disrupts the ATPase function of the myosin-IIA. In embodiments, the mutation is selected from the group consisting of A454V, E457K, E465Q, N470S, E530K, T538K, D567N, G696S, L812X, E1131M, S1163X, K1249E, F1261L, A1351P, L1411P, L1485P, and combinations thereof. Testing the sample in certain embodiments comprises determining a polynucleotide sequence of the Myh9 gene by use of any of a variety of amplification reactions that include use of synthetic DNA primers and/or the formation of cDNA and amplification reactions that comprise cDNA segments. In embodiments, testing the sample comprises detecting a complex of a detectably labeled agent, such as an antibody, which is specifically hybridized to a MYH9 protein comprising one or more of the mutations. In embodiments, the aggressive cancer determined by the method is a squamous cell carcinoma of the head and neck or a skin cancer or a breast cancer.

In another aspect the disclosure includes a method for identifying an individual as a candidate for treatment with a nuclear export inhibitor comprising testing a sample of a tumor from the subject to determine a mutation in the Myh9 gene and/or low expression of the Myh9 gene relative to a reference, wherein the presence of the mutation in the Myh9 and/or the low expression of the Myh9 gene relative to a reference indicates that the individual is a candidate for therapy with a nuclear export inhibitor.

In another aspect the disclosure includes a method for determining whether tumor cells have defective p53 nuclear transportation comprising testing tumor cells for a mutation in the Myh9 gene, wherein the presence of the mutation in the Myh9 gene determines that the cells have defective p53 nuclear transportation.

In another aspect the disclosure includes a method for treating an individual diagnosed with an aggressive cancer, wherein the aggressive cancer comprises cancer cells which comprise a mutation in the Myh9 gene. The method of treating comprises administering to the individual a composition comprising an effective amount of a nuclear export inhibitor.

### BRIEF DESCRIPTION OF THE FIGURES

The patent or application file contains at least one drawing executed in color. Copies of this patent or patent application publication with color drawing(s) will be provided by the Office upon request and payment of the necessary fee.

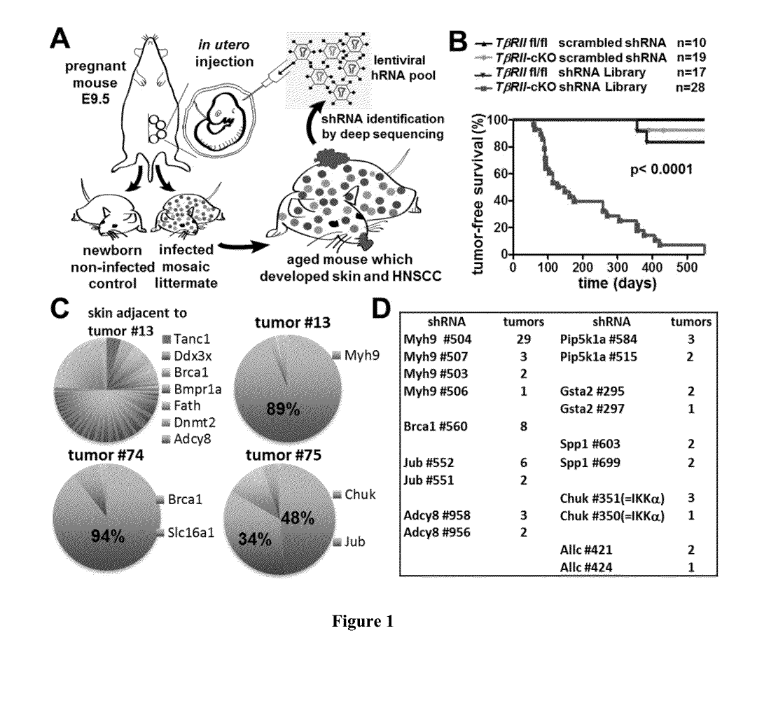

FIG. 1 shows an example of direct in vivo shRNA screen for HNSCC tumor suppressors (A) Schematic of the pooled shRNA screen. (B) Tumor-free survival for mice of the indicated genotype transduced at E9.5 with shRNA library targeting putative HNSCC genes. (n=number per group; p<0.0001, log-rank test) (C) Representative pie charts compiling DNA-sequencing analyses of individual tumors compared to surrounding healthy skin. Charts show % representation of a particular shRNA relative to the total. (D) The nine top-scoring tumor-suppressor candidates and corresponding tumor numbers in which their shRNAs were found to be significantly enriched.

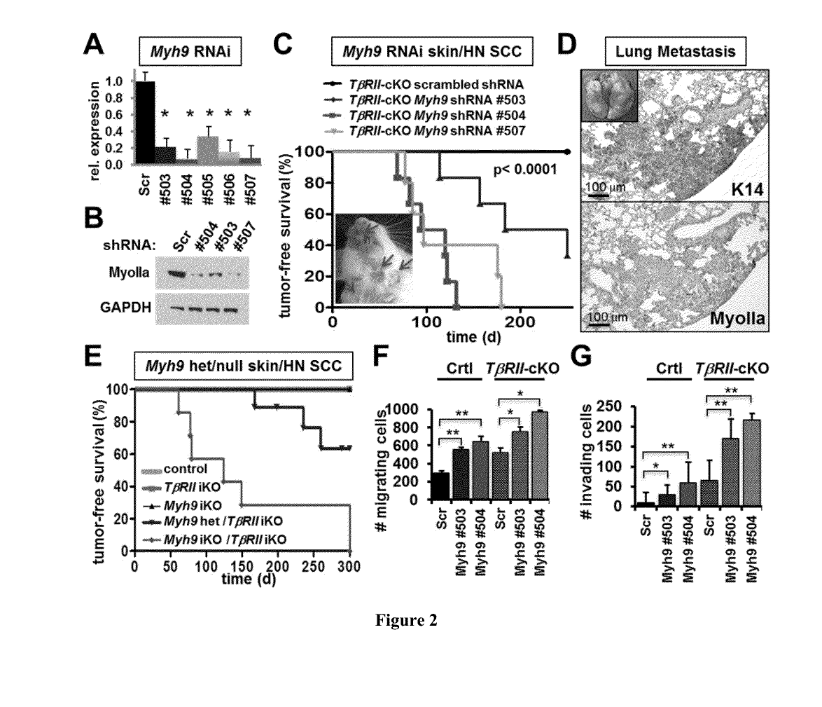

FIG. 2 shows an example of functional validation of Myh9 as a bona fide tumor suppressor and regulator of migration/invasion. (A) Quantitative RT-PCR of Myh9 mRNA in cultured primary murine keratinocytes infected with various Myh9-shRNA lentiviruses. Values are normalized to scrambled-control shRNAs. (n=3±SEM *p<0.005) (B) Immunoblot analysis of protein lysates from epidermal keratinocytes of newborn mice transduced in utero with indicated Myh9-shRNAs. (C) Tumor-free survival of mice of the indicated genotype and shRNA transduction. (n=6 for each genotype, p<0.0001, log-rank test between scrambled- and each Myh9 shRNA-infected cohort). Insert shows numerous skin lesions (arrows) on 4 month-old Myh9-shRNA transduced TβRII-cKO mouse. Scale bars, 30 μm. (D) Myh9-knockdown results in widespread pulmonary SCC metastases in and around blood vessels in the lungs of TβRII-cKO mice. Of note, metastasic lesions are immunoreactive for epithelial keratin 14 and negative for myosin-IIa. (E) Tumor-free survival of Myh9/TβRII inducible knockout (iKO) as well as Myh9 heterozygous/TβRII iKO and control mice (n=6,p<0.001, log-rank test). (F and G) Transwell migration assays through Boyden chambers coated with (F) fibronectin (migration assay) or with (G) Matrigel ECM (invasion assay). Myh9-deficiency markedly increases migration and invasion towards fibroblast-conditioned medium (bottom chamber), irrespective of TβRII-cKO status. (n=3±SEM *p<0.05 and **p<0.005, two-tailed t test between scrambled and each Myh9-knockdown construct).

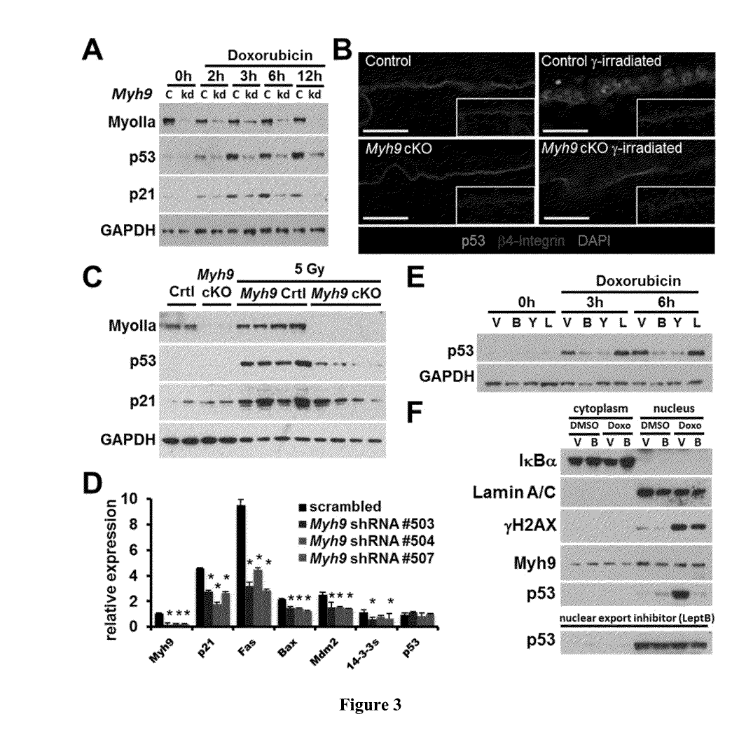

FIG. 3 demonstrates a representative non-canonical role for myosin-IIa in nuclear retention of activated p53. (A) Myh9-shRNA knockdown (kd) but not scrambled-shRNA shRNA control (c) diminishes p53 activation and target p21 expression in response to DNA-damage-response inducer doxorubicin (1 μM). Myosin-IIa and GAPDH levels are shown as controls. (B and C) Lack of nuclear p53 in Myh9-cKO versus control (Ctrl) littermate skins 6 hours after γ-irradiation (5Gy). (B) Immunofluorescence; (C) immunoblot analysis. Myosin-IIa and GAPDH levels are shown as controls. (D) qPCR of p53 target gene transcripts illustrate the relative magnitude of the effects of Myh9-knockdown on the p53 pathway. (E) p53 immunoblot of lysates from DDR-induced keratinocytes treated with vehicle (V), blebbistatin (B), Rock inhibitor Y27632 (Y) or latrunculinB (L). GAPDH levels are shown as controls. (F) Nuclear p53 is not retained when DDR-induced Myh9-knockdown primary keratinocytes are exposed to blebbistatin (B). Lamin A/C, IκBα and H2AXγ are controls for nuclear, cytoplasmic fractions and DDR, respectively. Nuclear export inhibitor Leptomycin B rescues the ability of Myh9-deficient cells to retain p53 in the nucleus.

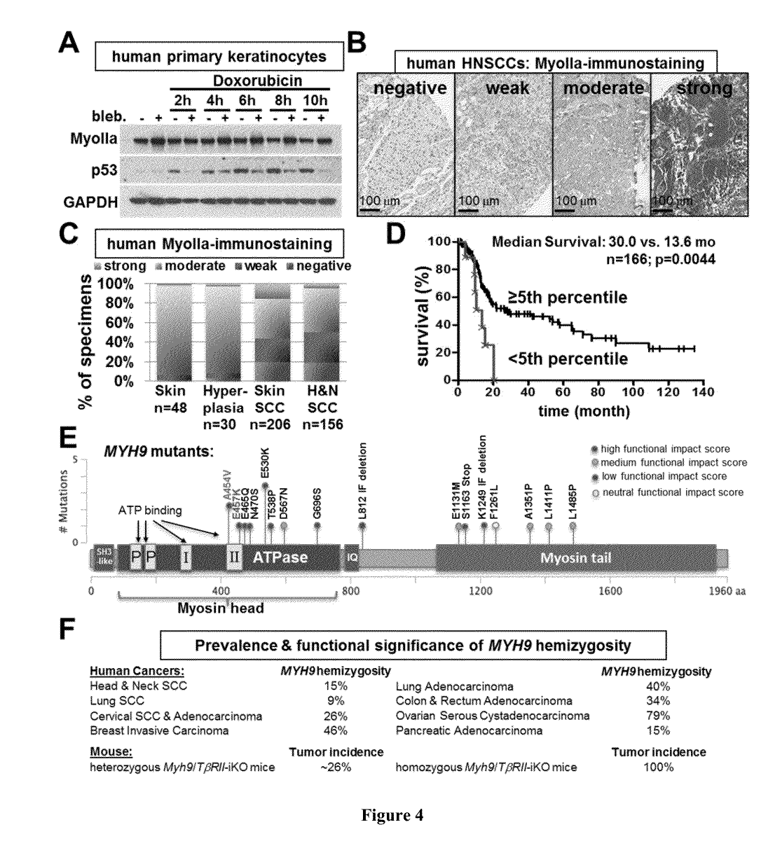

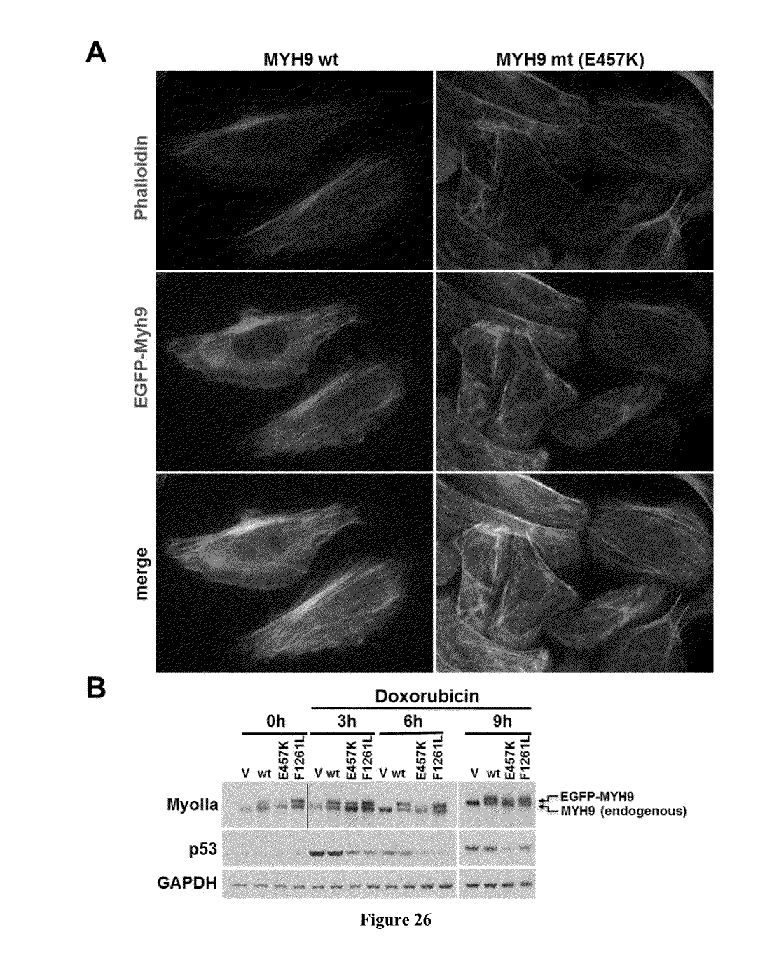

FIG. 4 shows MYH9 is a bona fide tumor suppressor in human HNSCC. (A) p53 induction in primary human keratinocytes treated with myosin ATPase inhibitor blebbistatin (4μM) and with DDR-inducer doxorubicin (Dox; 1 μM). GAPDH, loading control. (B) Representative images of myosinIIa-immunostained human HNSCCs displaying negative, weak, moderate or strong staining patterns. (C) Myosin-IIa quantifications on human healthy skins, skin SCCs and HNSCCs (n=362 patient samples analyzed). Note that a substantial fraction of cases show absent or reduced myosin-IIa. (D) Decreased MYH9 expression correlates with shortened survival. Kaplan-Meier analysis comparing overall survival of TCGA HNSCC patients partitioned according to the lowest (<5^{th} percentile) MYH9 expression versus the rest (≧5^{th} percentile) (n=166, p=0.0044, log-rank test). (E) Schematic of human myosin-IIa delineating the N-terminal SH3-like domain, the myosin head domain with the ATPase function, the ATP binding pockets P-loop (P) and switch region I and II (I and II), the IQ-calmodulin binding domain and the myosin tail. Missense mutations as well as deletions are given with their respective functional impact score overhead. Note that most of the mutations are within the ATPase domain clustering in and around the switch-II region (p=0.0015; Fisher test corrected for false discovery rate). Of note, mutations of the conserved A454 (blue) residue have been shown in Dictyostelium myosin to abrogate ATPase function. E457K (red) was tested and shown to have an effect on DDR-induced p53 activation (FIG. 26). (F) List delineating various cancer types with their respective percentage of MYH9 hemizygous loss as well as the percentage of Myh9 heterozygous and inducible knock-out mice that develop skin and/or head and neck SCCs on a TGFβRII-cKO backround (Myh9-dependent).

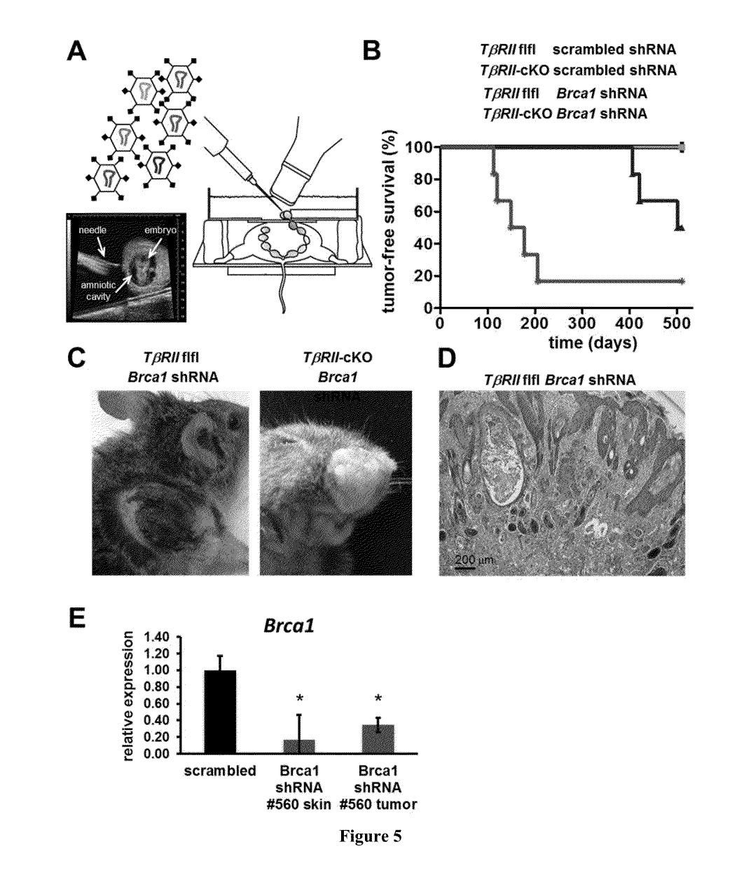

FIG. 5 shows an example of a strategy for using lentiviral-mediated in utero delivery of shRNAs to screen and study the effects of tumor suppressors on squamous cell carcinoma formation in vivo (A) Schematic to develop chimeric mice whose epidermis, glandular and oral epithelia are specifically, stably and clonally transduced with lentiviral construct harboring a fluorescently-tagged histone reporter gene and a desired shRNA driven by a U6 promoter. Non-invasive lentiviral infection of single-layered surface ectoderm is achieved by ultrasound-guided in utero injections into the amniotic sac of an E9.5 embryo (Beronja et al., 2010). (B) Kaplan-Meier analysis of tumor-free survival of mice of the indicated genotype transduced with an shRNA that efficiently targets Brcal-knockdown. (n=6 for each genotype, p<0.0062, log-rank test between TβRII-cKO vs. TβRII fl/fl mice infected with shRNA targeting Brcal). Note that on a TβRII-cKO background, Brcal shRNA-mediated initiation of tumor growth is greatly accelerated. (C) Representative images of Brcal shRNA transduced TβRII fl/fl and TβRII-cKO mice showing lesions on backskin as well as in oral cavity, respectively. (D) Representative section of a Brcal knockdown tumor isolated from a TβRII fl/fl animal showing a well-differentiated SCC. (E) In vivo knockdown efficiency of Brcal shRNA #560 in skin and in SCC tumors as measured by quantitative RT-PCR. (n=3±SEM *p<0.05).

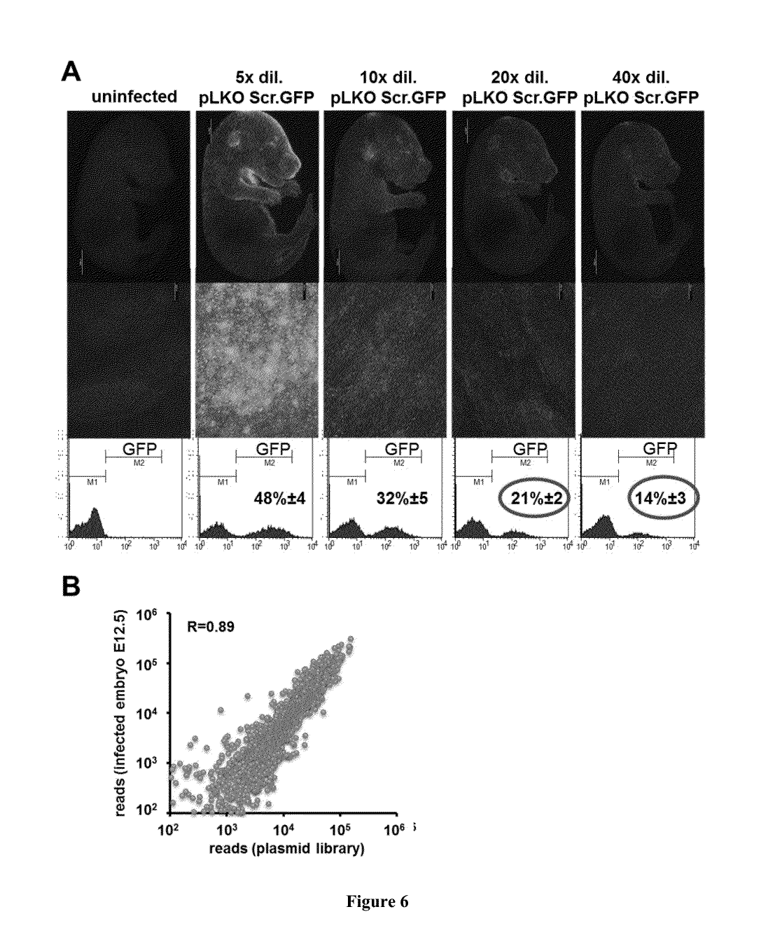

FIG. 6 shows an example of determining suitable viral titer and measuring lentiviral shRNA library representation. (A) Control lentivirus (pLKO), harboring an H2B-GFP reporter transgene and a U6-driven scrambled shRNA control (Scr) expression vector was used in a dilution series to determine the appropriate dilution/titer required to selectively and stably transduce about 15-20% of surface ectoderm keratinocytes in vivo by ultrasound-guided in utero delivery to the amniotic sacs of living E9.5 embryos. Fluorescence activated cell sorting (FACS) analyses of epidermal keratinocytes isolated from transduced pups at E18.5 were used for quantifications. Comparative quantitative RT-PCR was then used to estimate the required dilution of the test lentiviral shRNA library needed to give rise to 15-20% of infection (not shown). Control lentivirus as well as the test lentiviral shRNA library had an initial titer of ˜6×10⁹ cfu/ml and were diluted 40× for all subsequent infections. (B) Scatter plot of Illumina sequencing data, illustrating good correlation between the number of reads per shRNA in DNA isolated from the lentiviral plasmid library versus the actual shRNA representation in DNA isolated from transduced epidermal keratinocytes of mouse embryos 3 days after infection with the lentiviral library (R=non-parametric (Spearman) correlation coefficient).

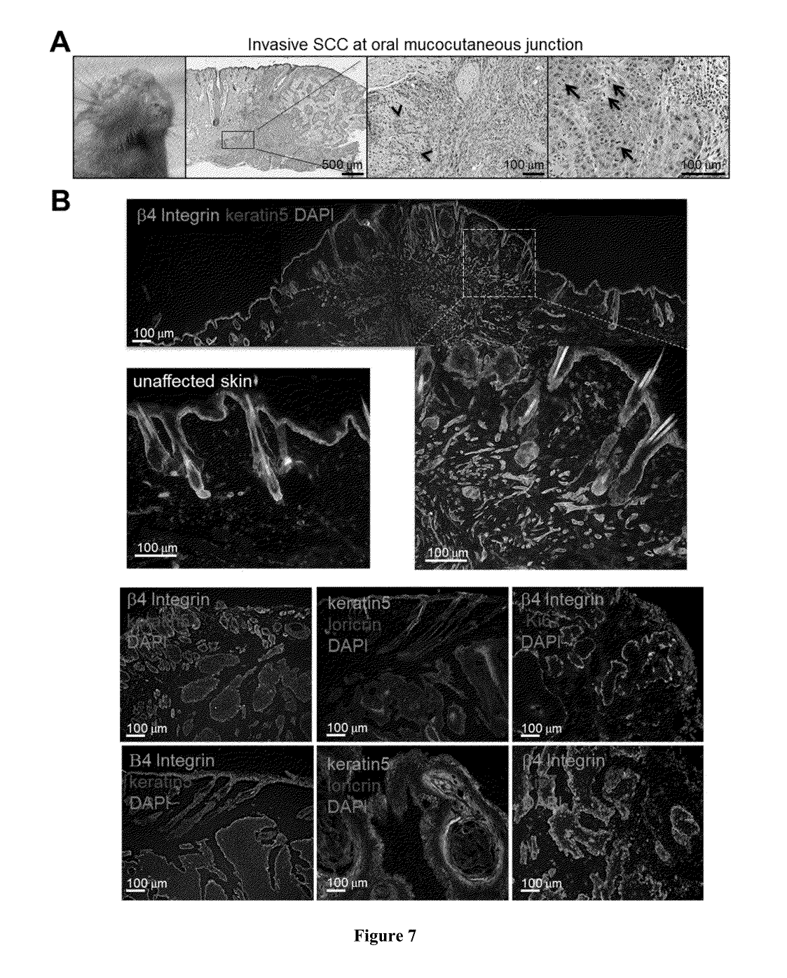

FIG. 7 shows an example of SCC formation in TβRII-cKO mice infected with the shRNA library (A) Histological sections of invasive SCC from oral cavity/lip of a transduced TβRII-cKO mouse. Different magnifications accentuate tumor heterogeneity, with well-differentiated areas (typified by keratin pearls) adjacent to poorly-differentiated areas. Note invasion into subcutaneous muscle (arrowheads) as well as moderate atypia characterized by anisokaryosis and anisocytosis, hyperchromasia, and frequent large and prominent nucleoli. Mitoses were on average 10× more frequent than the surrounding WT tissue (arrows). (B) Representative immunofluorescence analyses for basal markers Keratin 5 and β4-integrin, differentiation marker Loricin, and proliferation marker Ki67 on tumor sections from adult TβRII-cKO mice that had been infected with the lentiviral shRNA library at E9.5 in utero.

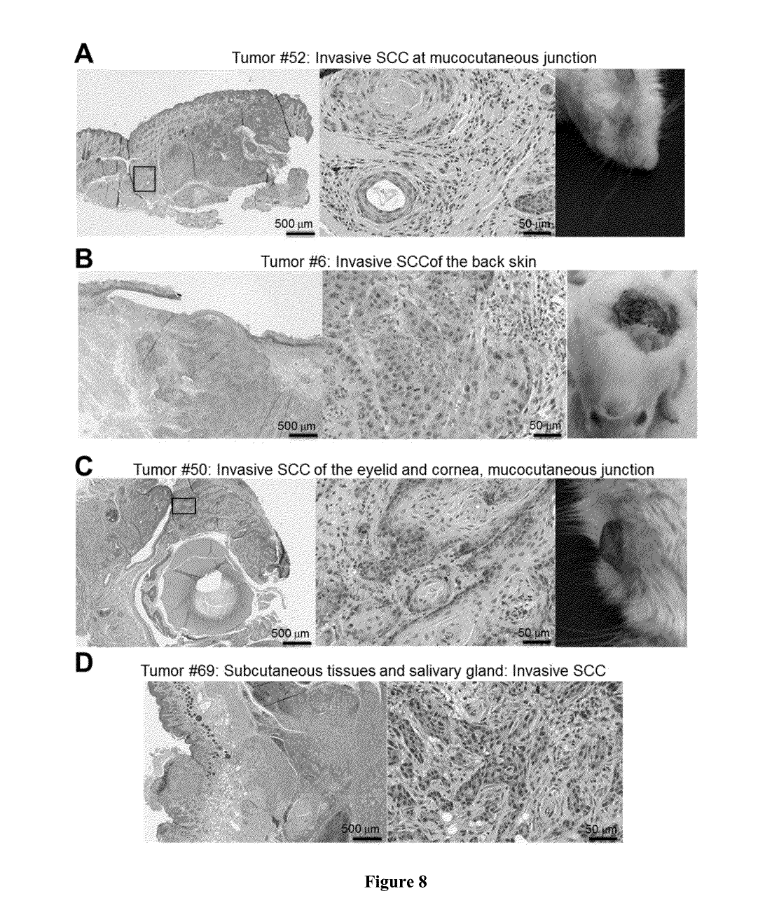

FIG. 8 shows an example of SCC formation in adult TβRII-cKO mice derived from embryos whose surface ectoderm was infected with the shRNA library (A to D) Representative H&E images of tumor sections showing invasive SCC arising from various transduced epithelial tissues as indicated. (A) At the mucocutaneous junction, a poorly demarcated neoplasm has invaded the dermis. The SCC is composed of nests and cords of basal cells exhibiting signs of squamous differentiation, notably eosinophilic keratin pearls. Some nests show evidence of stroma invasion associated with a desmoplastic stroma. Cellular atypia are minimal and mitoses are not observed within well-differentiated areas. The overlying epidermis is moderately hyperplastic and hyperkeratotic. The tumor is infiltrated by numerous neutrophils. (B) Backskin squamous cell carcinoma invading the underlying dermis and subcutaneous tissue. The SCC is well-demarcated, but in several areas, cells have detached from the main tumor and invaded into subcutaneous tissues. Invasive regions are characterized by small nests and cords of basal cells that have broken through the basement membrane and invaded adjacent stroma and muscle. This contrasts with nests of well-differentiated stratified squamous epithelium in the infundibular regions that are replete with keratinization. Throughout the tumor are scattered moderate to marked atypia characterized by fourfold anisokaryosis and anisocytosis, hyperchromasia, and variation in nucleolar size with frequent large and prominent nucleoli. Mitoses are prevalent at ˜38/ten 400× fields. (C) In this example, both cornea and eyelid are enlarged and their architecture is distorted by a poorly demarcated neoplasm composed of nests and cord of basal cells showing squamous differentiation and formation of keratin pearls. Some nests show evidence of stromal invasion associated with a desmoplastic stroma. Cellular atypia are minimal and mitoses are not observed. The overlying epidermis is moderately hyperplastic and hyperkeratotic. The tumor is infiltrated by numerous neutrophils. The cornea and conjunctiva are infiltrated by numerous neutrophils. In one eye, the lens is present in the section and shows swelling and liquefaction of lens fibers and posterior migration of lens epithelium. These tumors were often large, with involvement of both cornea and eyelids. The conjunctivitis and keratitis are ocular changes that appear to be secondary to expansion of the eyelid. (D) An SCC that has invaded subcutaneous tissues and the salivary gland. The tumor is a poorly demarcated and infiltrative neoplasm, composed of basal-like cells forming nests and cords supported by desmoplastic stroma. Cells are polygonal, have indistinct borders, and display a moderate amount of eosinophilic cytoplasm. They have ovoid nuclei with finely stippled chromatin and small nucleoli. There is threefold anisokaryosis, and an average of 12 mitoses per 400× fields. The skin shows a focally extensive area of epidermal hyperplasia, with focal epidermal ulceration with serocellular crusting. The dermis is infiltrated by moderate numbers of neutrophils and macrophages, and fewer lymphocytes.

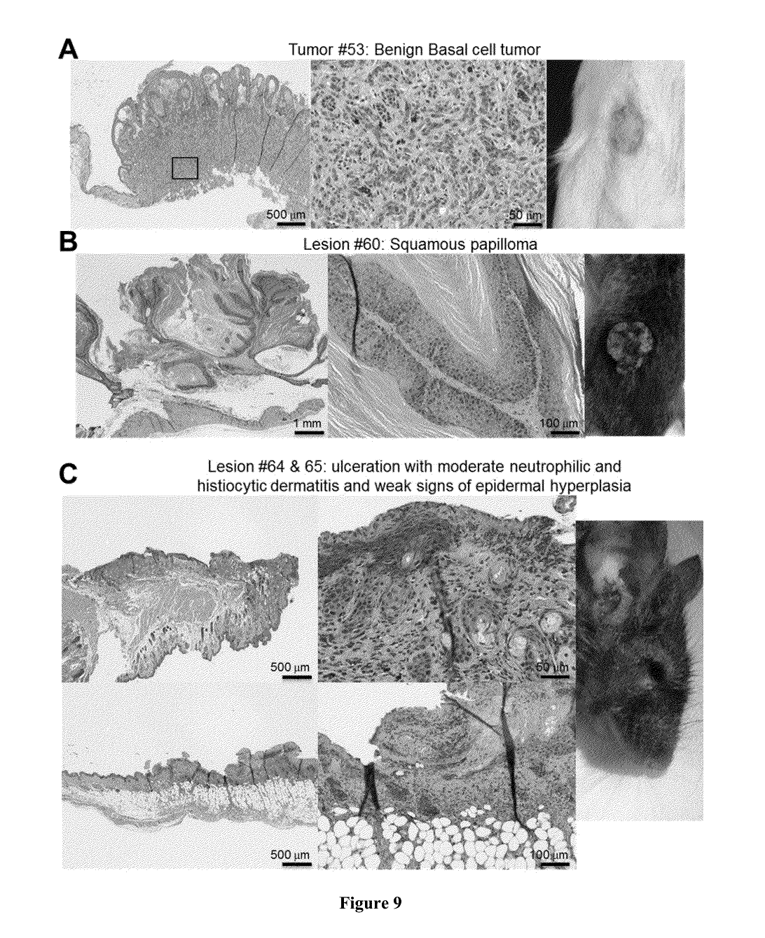

FIG. 9 shows an example of formation of benign lesions in TβRII-cKO mice derived from embryos whose surface ectoderm was infected with the shRNA library (A to C) Representative H&E images of sections from affected TβRII-cKO epithelial tissues of mice that were transduced as embryos with the lentiviral shRNA library. (A) Neoplasm of basal cell tumor that appeared to be benign based on histologic features. Note the well-demarcated epidermal neoplasm that extends deep into the underlying dermis. It is composed of thin cords and nests of basaloid cells surrounded by fibrous stroma. Epithelial cells display indistinct borders, a small amount of amphophilic cytoplasm, and oval nuclei with finely stippled chromatin and multiple small nucleoli. An average of 3 mitoses were seen for every ten 400× fields. Overlying epidermis and infundibular epithelium show moderate hyperplasia and orthokeratotic hyperkeratosis. A few mm from this tumor is a well-demarcated region of deep dermal and subcutaneous fibrosis. (B) This squamous papilloma displays an exophytic, well demarcated neoplasm, composed of a branching papillary structure and markedly proliferative, but well differentiated, epidermis. Note marked orthokeratotic hyperkeratosis supported by thin stalks of fibrovascular stroma. The proliferative epidermis shows occasional mild dysplasia. The stroma is focally infiltrated by moderate numbers of melanophages and/or melanocytes, and moderate numbers of lymphocytes. (C) Some lesions showed no signs of malignancy. In this example, only ulceration are seen, with moderate neutrophilic and histiocytic dermatitis and weak signs of epidermal hyperplasia, indicating that these lesions are likely to be preneoplastic. Note focally extensive areas of mild epidermal hyperplasia, with multifocal epidermal ulceration associated and serocellular crusting and dermal necrosis. The superficial, mid and deep dermis is multifocally infiltrated by small to moderate numbers of neutrophils and macrophages, and fewer lymphocytes.

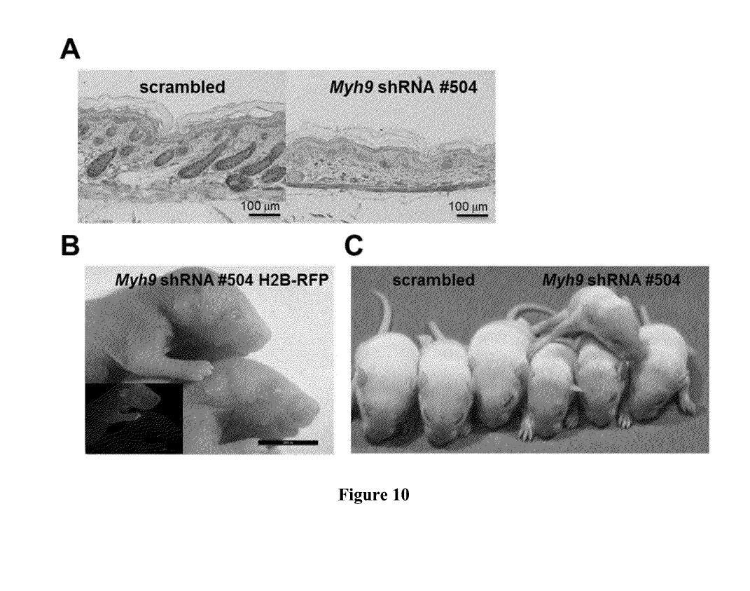

FIG. 10 shows an example of how Myh9 knockdown delays hair follicle downgrowth and impedes eyelid closure. Mice were transduced at E9.5 with scrambled-control or Myh9 #504 shRNAs, and examined at birth. (A) Myosin-IIa immunohistochemistry of skin sections from these mice. Note loss of myosin-IIa and impaired hair follicle down-growth in Myh9 knockdown animals. (B) Newborn mice reveal “Open Eyes at Birth” phenotype indicative of an impediment to eyelid closure during embryonic development. Inset shows that mice were efficiently transduced with the lentivirus, as judged by expression of the reporter H2B-RFP fusion protein. (C) 8 day-old Myh9 shRNA-transduced mice show sparse and delayed hair growth compared to scrambled infected littermate controls.

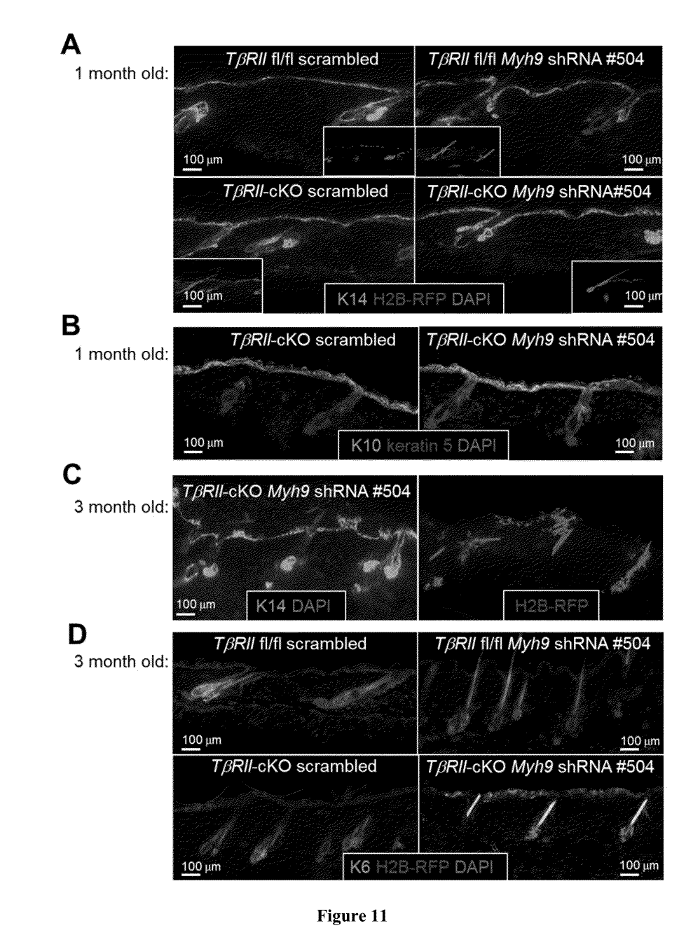

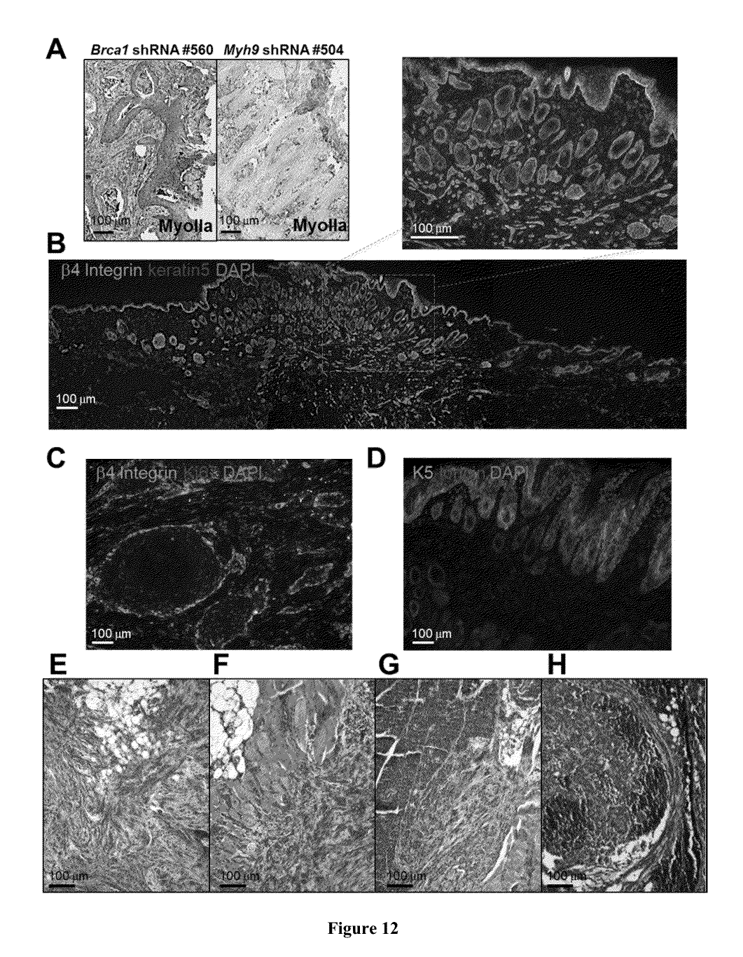

FIG. 11 shows an example of how Myh9 knockdown does not interfere with tissue homeostasis in skin in young animals (A to D) Fluorescence microscopy of frozen skin sections from Myh9 knockdown, TβRII-cKO and TβRII fl/fl mice at one (A and B) or three (C and D) months of age. Mice had been transduced in utero at E9.5 with lentivirus expressing an H2B-RFP reporter and either Myh9 #504 or scrambled shRNAs. Note that transduced regions (RFP+) show grossly normal immunolabeling for (A) Keratin 14 in the basal cells of interfollicular epidermis and hair follicles, and (B) K10, specific for terminally differentiating epidermis. In older animals, sparse areas of epithelial thickening were noted, concomitant with expanded K14 expression (C) and induction of K6, associated with a hyperproliferative state (D).

FIG. 12 shows a representative validation of Myh9 as a tumor suppressor (A) Sections of tumors from TβRII-cKO mice, transduced with shRNAs targeting Brcal or Myh9, respectively, and immunolabeled for myosin-IIa (absent in the epithelium of Myh9 #504 shRNA-targeted SCCs). (B to D) Immunofluorescence microscopy of frozen tissue sections from tumors arising spontaneously in TβRII-cKO mice that had been transduced as embryos with Myh9 shRNAs. Note architecture of poorly differentiated SCCs with (B) β4-integrin and K5-expressing nodules, (C) high proliferation rates in the basal layer as indicated by nuclear Ki67 and (D) reduced expression of differentiation markers such as Loricin. (E to H) H&E of paraffin sections of these tumors confirmed their identity as poorly differentiated squamous cell carcinomas that invade into (E) subcutaneous fat, (F) skeletal muscle, (G) salivary gland and (H) locally draining lymph node.

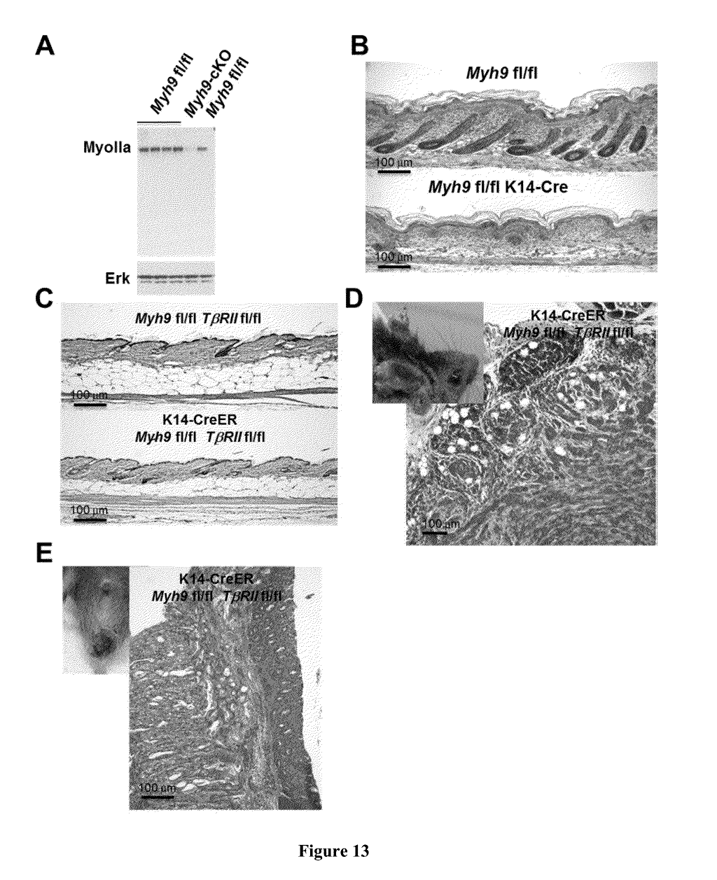

FIG. 13 shows an example of genetic ablation of Myh9 phenocopies Myh9 shRNA knock-down (A) Western Blot analysis of keratinocytes purified from Myh9 fl/fl K14-Cre (Myh9-cKO) mice and control littermates show target-specific reduced expression of myosin-IIa. (B) Anti-myosin-IIa immunolabeling of skin sections of wild-type and K14-Cre conditionally targeted Myh9-cKO animals. Note the antibody specificity and the recapitulation of the impediment to hair follicle down-growth, also seen with Myh9 knock-down. (C) Histology of skin sections of double mutant (Myh9/TβRII iKO) mice inducing K14-driven with topical application of tamoxifen (D) Representative Myh9/TβRII iKO animal as well as H&E section showing a poorly differentiated skin SCC that has invaded through the skeletal muscle into the deep subcutaneous structures and lymph nodes. (E) Representative Myh9/TβRII iKO animal as well as H&E section showing a moderately differentiated invasive anogenital squamous cell carcinoma that has invaded the colonic epithelium. The colonic epithelium is not neoplastic, but is ulcerated and inflamed with some reactive changes.

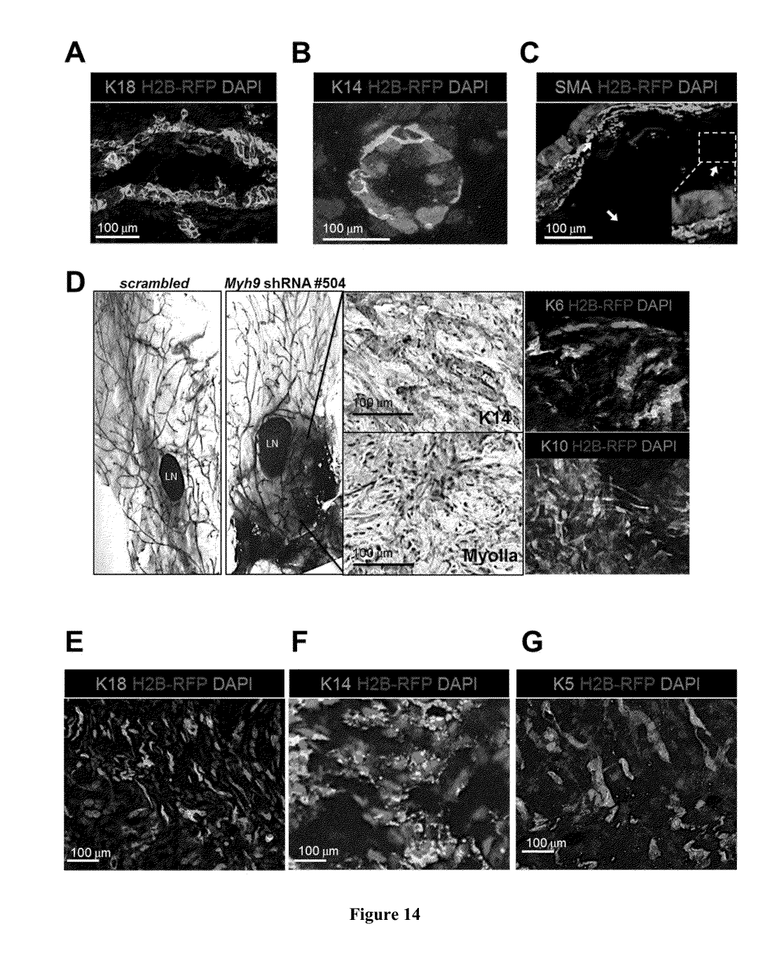

FIG. 14 shows an example of TβRII-cKO mice transduced with Myh9 shRNA develop multiple SCCs in the mammary gland (A to C) In utero infections of E9.5 surface ectoderm results in appreciable transduction of mammary epithelial tissues. Epifluorescence and immunolabeling of frozen tissue sections of transduced mammary epithelium. Transduced areas (H2B-RFP+) include (A) luminal epithelium (K18+) and (B and C) myoepithelium (positive for K14 and smooth muscle actin). (D) Whole-mount of 12-week-old scrambled and Myh9 shRNA-transduced TβRII-cKO mammary gland. LN, lymph node. Arrows denote neoplastic regions that were subjected to immunolabelings at right. Mammary SCCs were positive for K14, K6 and K10 as well as H2BRFP (denoting transduced cells, negative for myosin-IIa). (E to G) Immunofluorescence of SCC lesion from mammary tissue of TβRII-cKO mice transduced with Myh9 shRNA #504. Note co-localization of lentiviral reporter H2B-RFP and luminal markers (E) keratin 18 (K18) and basal keratins (F) K14 and (G) K5.

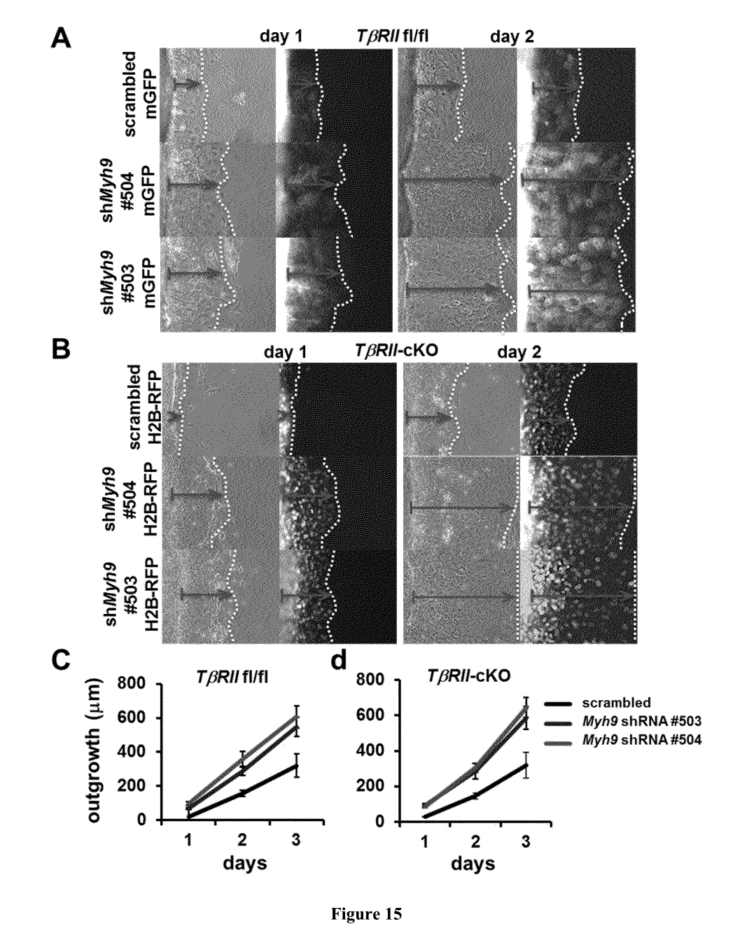

FIG. 15 shows an example of how Myh9 regulates epidermal outgrowth from skin explants (A and B) Representative phase-contrast and epifluorescence images of (A) TβRII fl/fl and (B) TβRII-cKO skin explants from E18.5 embryos infected at E9.5 with scrambled-control or shRNAs construct targeting Myh9. Viral constructs harbored reporter genes encoding either membranous GFP (mGFP) or H2B-RFP. Epidermal outgrowth was monitored for 48 hr and was significantly increased in Myh9 shRNA-transduced keratinocytes compared to scrambled control transduced explants of TβRII-proficient and deficient cells. White dotted lines mark leading edges; red arrows denote distance between explant and its leading edge. (C and D) Quantifications of epidermal outgrowth from skin explants of (C) TβRII fl/fl and (D) TβRII-cKO mice transduced with indicated knock-down constructs. (n=3±SEM *p<0.05, two-tailed t test between scrambled and each Myh9 knock-down construct)

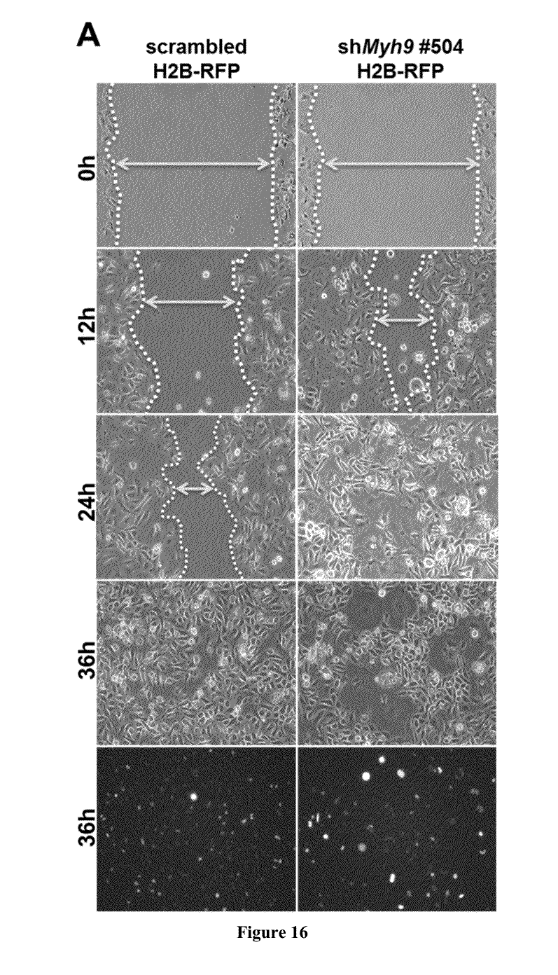

FIG. 16 shows an example of how Myh9 knockdown enhances keratinocyte migration in a scratch wound assay in vitro. (A) Shown are representative temporal phase-contrast and RFP epifluorescence images of scratch wound assays on keratinocytes infected in vitro with scrambled-control or Myh9 shRNAs #504. Yellow arrows indicate the extent of wound closure. H2B-RFP marks transduced keratinocytes as shown in the last panel.

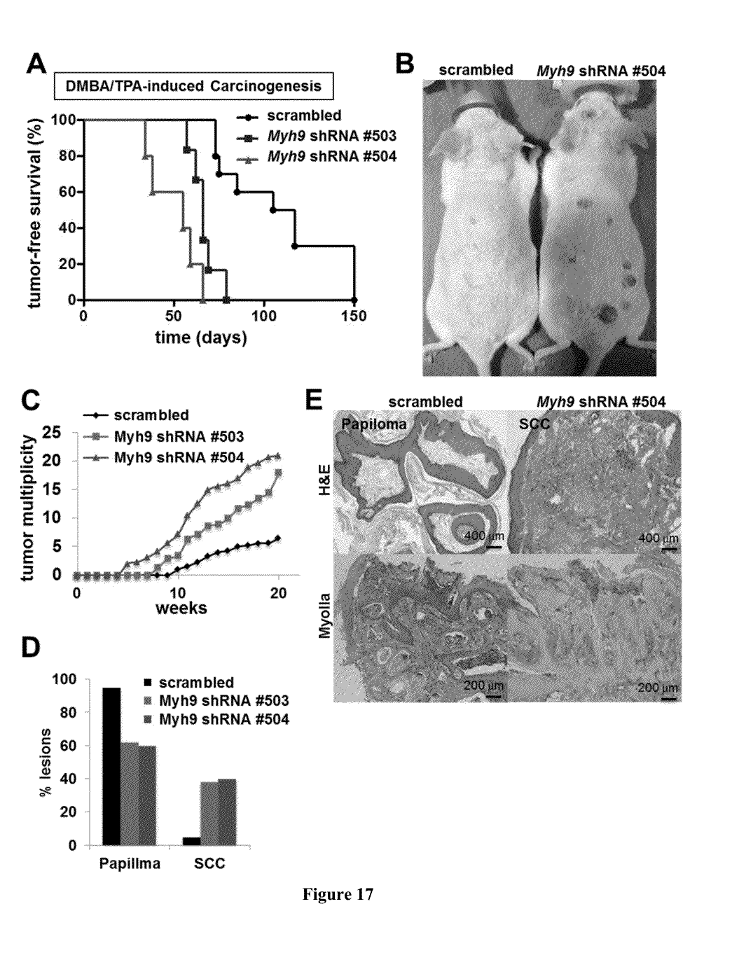

FIG. 17 shows an example of how Myh9 regulates Ha-Ras-driven tumorigenesis (A) Kaplan-Meier analysis of tumor-free survival of DMBA/TPA treated (Ha-Ras-induced) syngenic CD1 mice transduced with the indicated shRNA. (n=6 for each genotype, p<0.0005, log-rank test between scrambled control and each Myh9 shRNA infected mice). (B) Representative images of CD1 mice, transduced in utero with either scrambled control or Myh9 shRNAs #504, and 12-weeks after DMBA-treatment. (C) Tumor multiplicity of DMBA/TPA treated CD1 mice transduced with the indicated shRNA. (n=6 for each genotype). (D) SCC conversion frequency in syngenic CD1 mice transduced with the indicated shRNA 20-weeks after DMBA-treatment. (E) Representative H&E as well as MyoIIa IHC images of tumors from CD1 mice transduced in utero with either scrambled control or Myh9 shRNAs #504 and 20-weeks after DMBA-treatment.

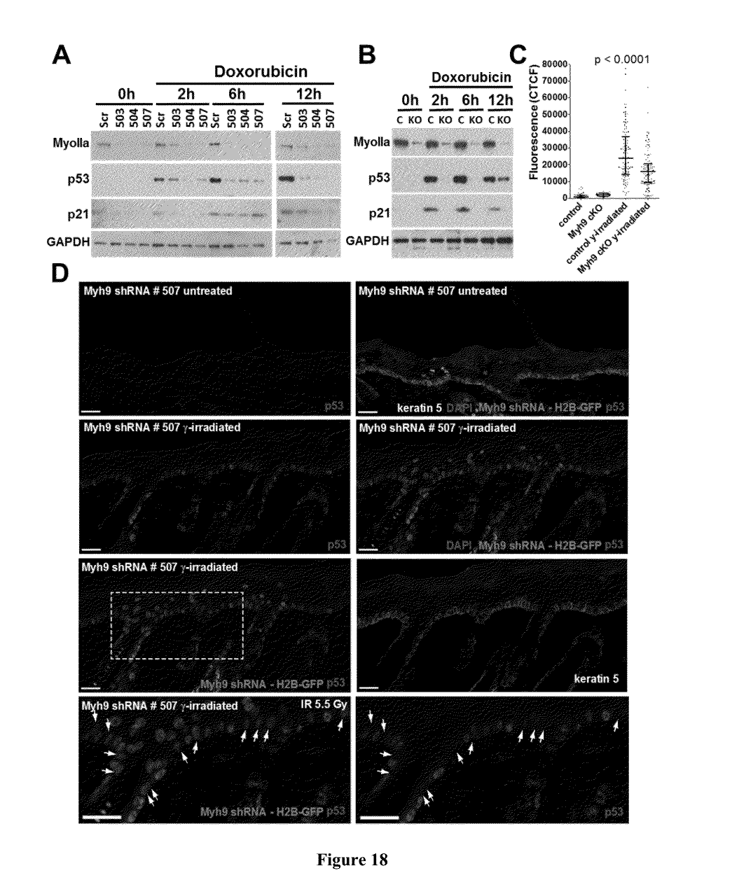

FIG. 18 shows an example of how Myh9 regulates p53. (A) p53 and p21 expression after treatment with DNA damage response drug doxorubicin (Dox; 1 μM). Primary mouse epidermal keratinocytes were transduced with the Myh9 shRNAs indicated. Myosin-IIa and GAPDH levels are indicated as control. (B) p53 and p21 expression after treatment with DNA-damage-response inducer doxorubicin (1 μM) Myh9fl/fl keratinocytes after adenoviral-Cre-mediated Myh9 ablation (KO). Myosin-IIa and GAPDH levels are shown as controls. (C) Quantification of p53 in nuclei of the skin of Myh9 cKO and control mice 6 hours after treatment g-irradiation (5Gy) as shown in FIG. 3B. Plotted is the corrected total cell fluorescence (CTCF) per cell and the median with interquartile range. (p<0.0001; Mann Whitney test). (D) p53 expression 6 hours after treatment g-irradiation (5Gy) in the skin of Myh9 knock-down (H2B-RFP labeled) mice. Note that p53 staining is only observed in basal keratin 5 positive cells. Note also that H2B-RFP labeled Myh9 shRNA #507-infected cells do not show efficient nuclear p53 staining Mosaic analysis shows that the mechanism involved is cell-intrinsic.

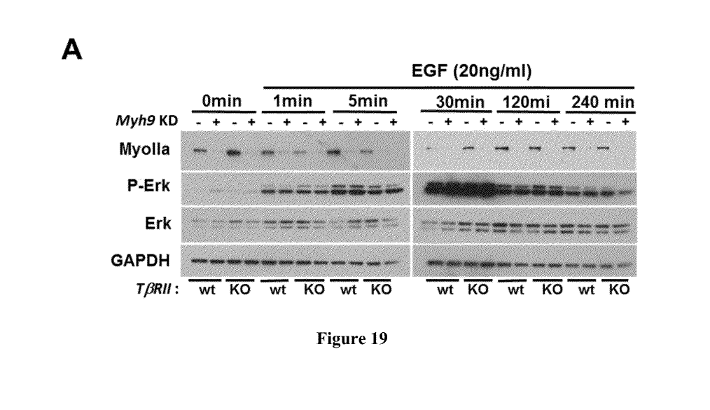

FIG. 19 shows an example of how Myh9 ablation does not affect EGF signaling. (A) Myh9 knockdown epidermal keratinocytes efficiently respond to EGF. Western Blot of phosphorylated (activated) Erk after EGF (20 ng/ml) stimulation of keratinocytes infected in vitro with various Myh9 knockdown constructs.

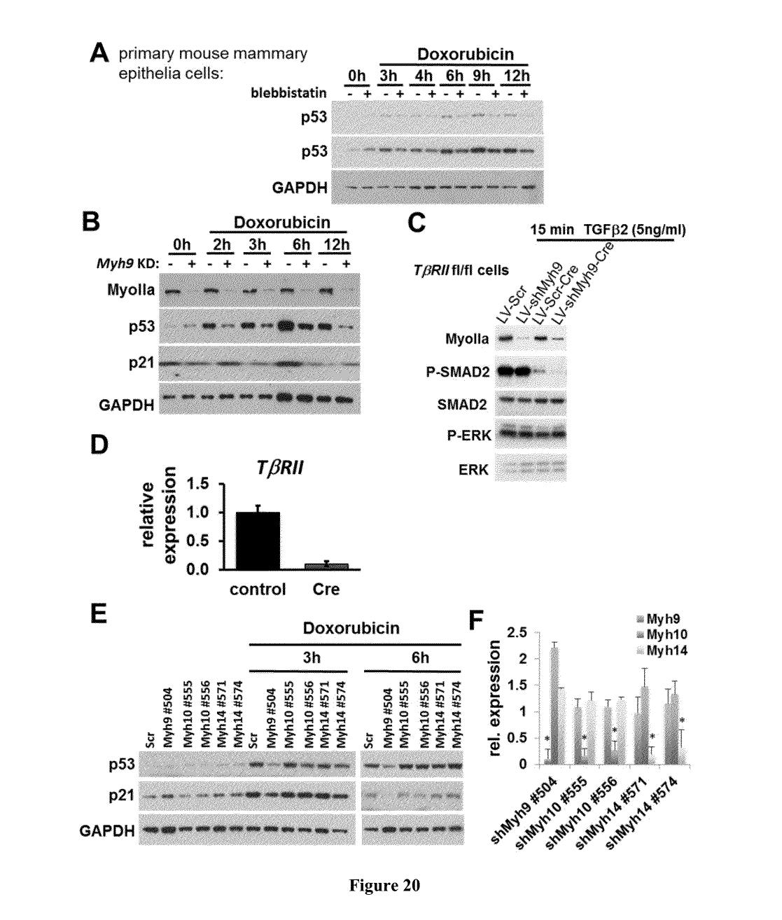

FIG. 20 shows an example of how Myh9 regulates p53 in TβRII-cKO keratinocytes and this effect is specific to Myh9. (A) p53 and p21 expression after doxorubicin (Dox; 1 μM) treatment of primary mammary epithelial cells. (B) p53 and p21 expression after doxorubicin (Dox; 1 μM) treatment of TβRII fl/fl keratinocytes transduced by lentiviral delivery of Myh9 shRNAs and Cre recombinase. Myosin-IIa and GAPDH levels are indicated as control. (C) Western Blot of phosphorylated (activated) P-SMAD2 in TβRII fl/fl keratinocytes transduced with indicated lentiviral constructs. Note that as expected, LV-Cre mediated targeting of TβRII resulted in loss of P-SMAD2 activity, which is downstream of TGFβ-signaling. Myosin-IIa, total SMAD2, activated phosphorylated P-ERK and total ERK are shown as controls. (D) qPCR analysis of TβRII to verify LV-Cre mediated ablation of TβRII gene expression. (E) p53 and p21 expression after treatment with DNA-damage-response inducer doxorubicin (1 μM) in wt keratinocytes after Myh9, Myh10 and Myh14 shRNA-mediated knockdown (kd). GAPDH levels are shown as controls. (F) qRT-PCR analysis of Myh9, Myh10 and Myh14 shRNA-mediated knockdown.

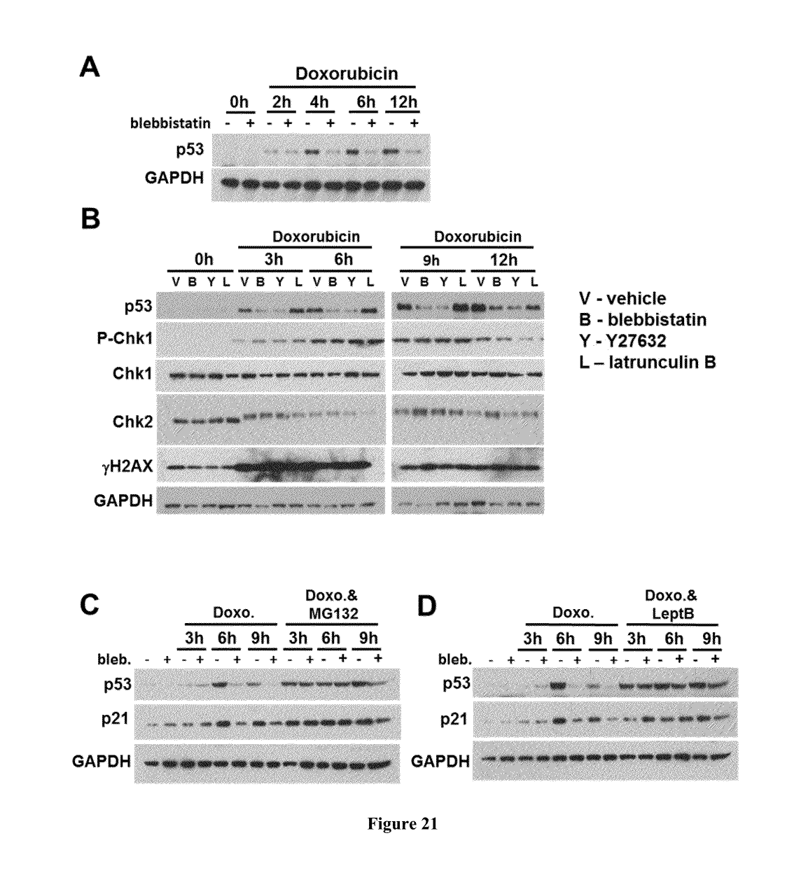

FIG. 21 shows representative optimal p53 activity following DNA damage depends upon myosin-IIa's ATPase activity and its role in p53 nuclear retention (A) p53 expression in mouse keratinocytes treated with myosin ATPase inhibitor blebbistatin (4 μM) and with doxorubicin (Dox; 1 μM). GAPDH levels are indicated as control. (B) Western Blot of p53 in keratinocytes treated with vehicle, blebbistatin, Rock inhibitor Y27632 or latrunculin B. Activated phosphorylated H2AX (γH2AX) as well as activated phosphorylated Chk1 and Chk2 shown normal DDR activation. Note activation-dependent mobility shift of Chk2. Total Chk1 and GAPDH are shown as controls. (C) MG132 rescues Myh9 phenotype (D) Nuclear export inhibitor Leptomycin B rescues the Myh9 knockdown phenotype and restores p53 accumulation after DNA damage.

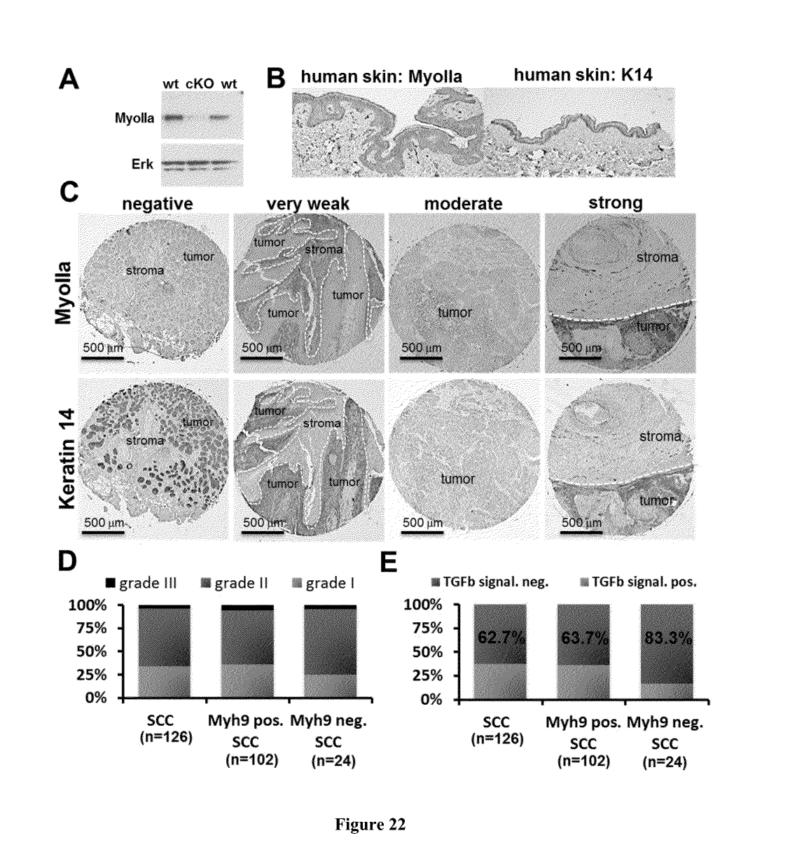

FIG. 22 shows a representative expression of myosin-IIa in human HNSCC and skin SCCs (A) Myosin-IIa Western Blot of primary Myh9-cKO keratinocytes to validate the efficacy of the myosin-IIa antibody. (B) Representative images of myosin-IIa immunohistochemistry of human HNSCCs. (C) Quantification of myosin-IIa staining in human skin, hyperblastic and HNSCC samples show variability in myosin-IIa staining ranging from negative to weak, moderate and strong. (D) Analysis of human skin SCCs with respect to tumor grading and then classified according to presence or absence of myosin-IIa expression. (E) Analysis of human skin SCCs with respect to absence or presence of TGFβ signaling as assessed by immunolabeling for TβRII and active P-SMAD2 and classified according to presence or absence of myosin-IIa expression.

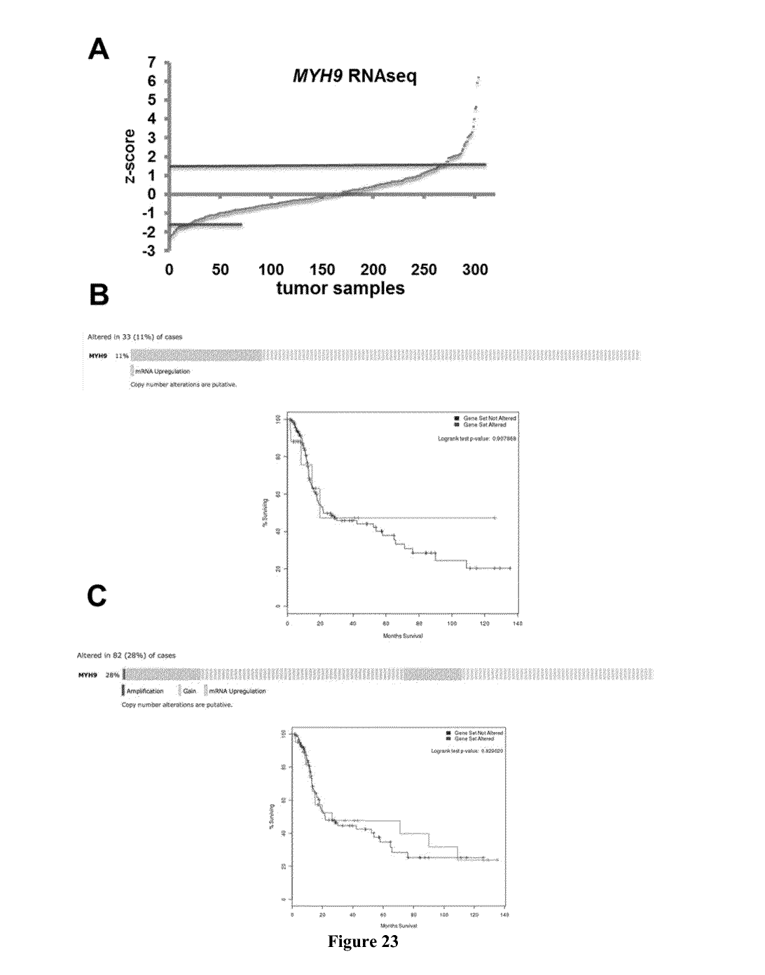

FIG. 23 demonstrates increased MYH9 expression does not impinge on human HNSCC survival (A) Raw RNAseq data of HNSCC samples in the TCGA database showing the spread of MYH9 RNA expression in all samples across the cohort. Graph delineates the z-score of MYH9 mRNA expression defined as the relative expression of an individual gene and tumor to the gene's expression distribution in a reference population, which is all tumors that are diploid for the gene in question. The returned value indicates the number of standard deviations away from the mean of expression in the reference population (z-score). This measure is useful to determine whether a gene is up- or down-regulated relative to the normal samples or all other tumor samples. In FIG. 4D and FIG. 23A we used the bottom 5th percentile, which equaled samples with a z-score of −1.6 or less (all samples below the red line) to perform the Kaplan-Meier survival analysis. Interestingly, this analysis also shows quite some HNSCC cases significant upregulation of MYH9 mRNA expression—top 33 patients (or top 11%) out of our cohort of 303 HNSCCs. (B) Kaplan-Meier survival analysis of of HNSCC cases with MYH9 mRNA upregulation (above 1.6 standart deviations or more indicated by the red line in FIG. 23A). In contrast to the data for the low MYH9 expression, these patients do not show any survival disadvantage/advantage when compared to the rest of the cohort. (C) Kaplan-Meier survival analysis of of HNSCC cases with MYH9 mRNA upregulation, MYH9 amplifications or gains. Of note, amplifications are defined as larger chromosomal amplifications while gains are defined as local amplifications.

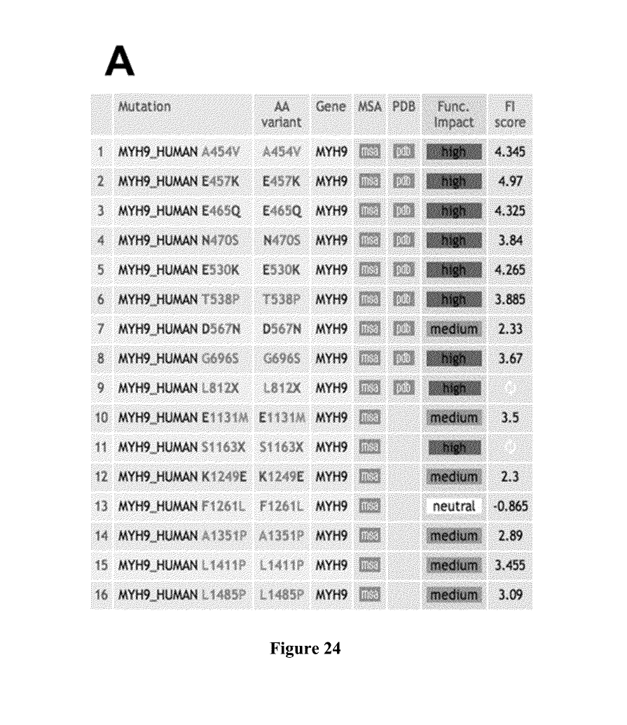

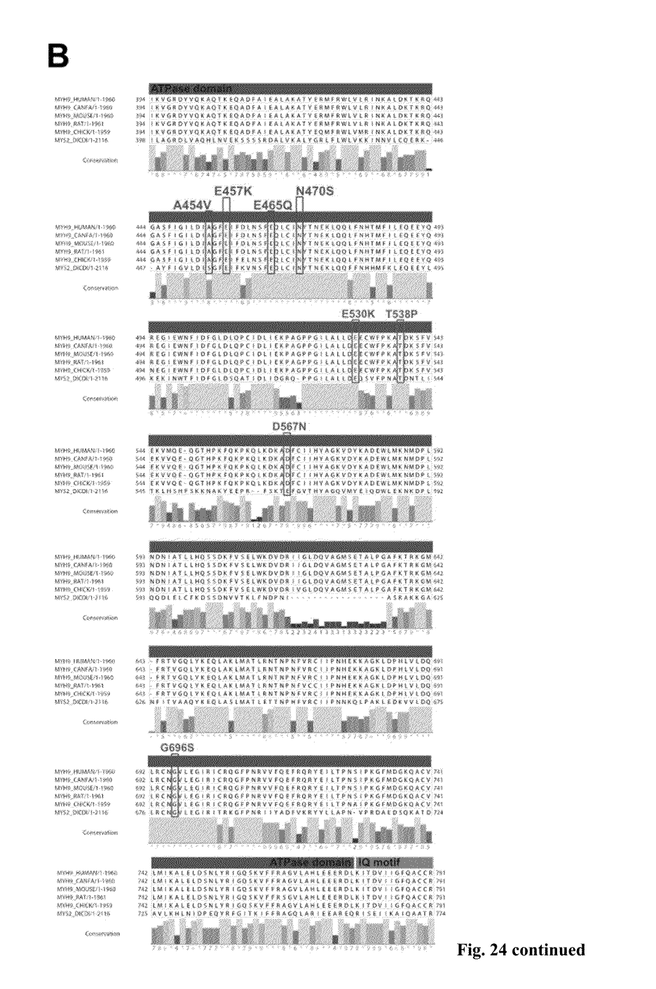

FIG. 24 shows an example of mutations in myosin-IIa in human HNSCCs (A) List of MYH9 mutations found in HNSCC and their computed functional impact score (www.mutationassessor.org). (B) Multiple sequence alignment of human, dog, mouse, rat, chicken MYH9 and Dyctyostelium discoideum (DICD) myosin-2 heavy chain from, top to bottom respectively. Multiple sequence alignment by MAFFT v7.058b (E-INSi strategy, Blosum 62, Offset value 0.123) and visualization using Jalview 2.8. The human sequence is SEQ ID NO:22; dog is SEQ ID NO:23; mouse is SEQ ID NO:24, rat is SEQ ID NO:25, chicken is SEQ ID NO:26, and Dyctyostelium discoideum is SEQ ID NO:27.

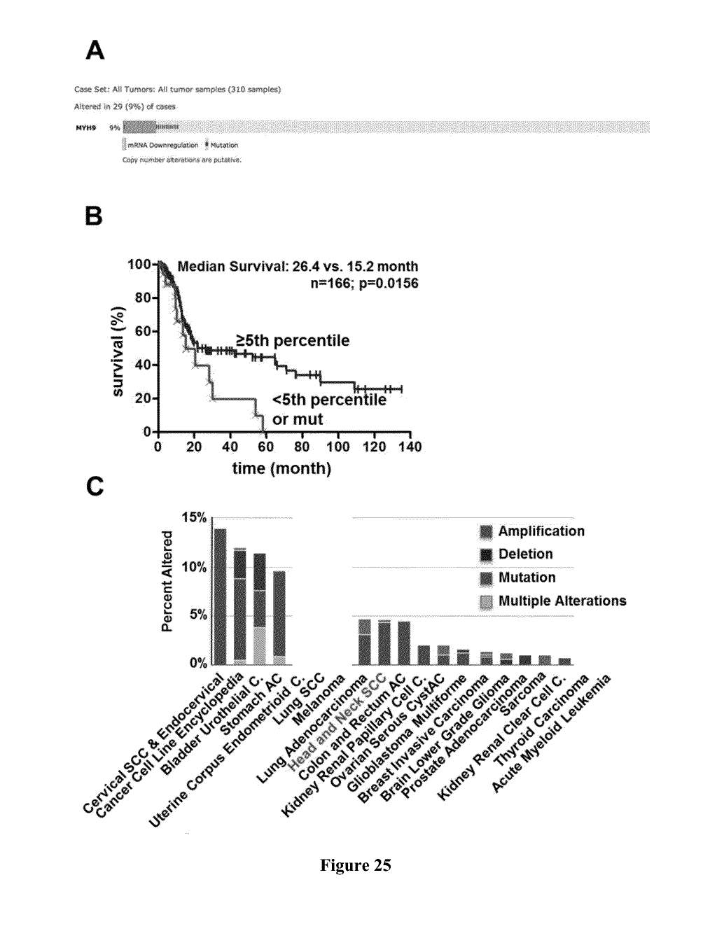

FIG. 25 shows a representative reduced MYH9 mRNA levels and presence of MYH9 somatic mutations correlate with HNSCC patients that show poor survival characteristics. Statistics shown were mined from the TCGA databases of 310 human HNSCC samples and their normal surrounding tissue controls. (A) Number of human HNSCC samples showing reduced MYH9 gene expression (˜5%) or somatic mutations within MYH9 (˜4%). Within 310 samples, 29 show alterations in MYH9 transcript levels. (B) Decreased MYH9 gene expression and MYH9 mutations together correlate with shortened survival. Kaplan-Meier analysis comparing overall survival of patients suffering from HNSCCs stratified by the lowest (<5th percentile) MYH9 expression and mutations in MYH9 versus the rest (>5th percentile and MYH9 wt). (n=166, p<0.0156, log-rank test) (C) Mutational spectrum of MYH9 across 19 human tumor types and 1000 human cancer cell lines (midified from cBioPortal: www.cbioportal.org/public-portal/).

FIG. 26 shows an example of mutations within the ATPase domain of MYH9 impair p53 activation. (A) Representative immunofluorescence images of phalloidin and anti-GFP stainined mouse keratinocytes expressing either wildtype human EGFP-MYH9 or mutant human EGFP-MYH9 (E457K). (B) p53 expression in primary mouse keratinocytes infectd with either vector control lentivirus or lentivirus harboring wildtype human EGFP-MYH9 (wt) or mutant human EGFP-MYH9 (E457K) or (S1261L) and treated with with doxorubicin (Dox; 1 μM). GAPDH levels are indicated as control.

### DETAILED DESCRIPTION

The present disclosure provides compositions and methods for making or for aiding in making a diagnosis of cancer, and for prophylaxis and/or therapy of certain types of cancer as described further below. In embodiments the disclosure provides methods for staging cancer, for making a prognosis for a subject diagnosed with cancer, for developing a personalized treatment protocol for an individual diagnosed with cancer, for making a diagnosis of an aggressive form of cancer, and therapeutic and/or prophylactic interventions for individuals diagnosed with or at risk for certain cancers, such as a risk of cancer recurrence. The disclosure relates to disruptions in the function of non-muscle myosin-IIA heavy chain. The non-muscle myosin-IIA heavy chain described herein is also referred to as “NMHCIIA” and “myosin-IIA.”

In general the disclosure is based at least in part on the present finding that mutations in the Myh9 gene in cancer cells affect the function of the non-muscle myosin-IIA heavy chain protein encoded by it and as a result, the cancer cells have a defect in the ability of p53 to accumulate in the nucleus, such as in the case of DNA damage-induced, post-transcriptional p53 activation. As a consequence, subjects who have mutations which affect the function and/or expression of mysosin-IIA have a worse prognosis and survival than those who do not have the mutations. Thus, the present disclosure reveals for the first time that mysosin-IIA has a tumor suppressor function which is pertinent to the etiology, diagnosis and therapy of a number of distinct cancer types.

In this disclosure we provide data demonstrating that chemical inhibition of nuclear export can rescue Myh9 mutations by enabling the cells which comprise defective mysosin-IIA to retain p53 in the nucleus. Accordingly, it is reasonable to expect that inhibition of nuclear export will provide a therapeutic and/or prophylactic benefit to individuals who harbor the Myh9 mutations described herein, and/or who otherwise have low levels of mysosin-IIA protein.

Without intending to be bound by any particular theory it is expected that the present disclosure will be pertinent to any cancer(s) that are correlated with and/or caused by defective mysosin-IIA activity such that the capability of p53 to accumulate in the nucleus is decreased. In embodiments, the cancer is a cancer of the oral cavity, a skin cancer, a mammary gland cancer, or a squamous cell carcinoma. In embodiments, the squamous cell carcinoma is a head and neck cancer. A significant enrichment for functional and truncating mutations has also be found in lung squamous cell carcinoma; colorectal carcinoma; cervical SCC & endocervical carcinoma; head and neck SCC; breast carcinoma; lung adenocarcinoma (see Table 3), thus in embodiments the disclosure is pertinent to any of these cancer types.

In one aspect, the method comprises testing a biological sample obtained from a subject for the presence or absence of a mutation that affects the function of mysosin-IIA. In embodiments, the mutation is any mutation that disrupts the ATPase function of the myosin-IIA. In embodiments, the mutation is selected from the group consisting of A454V, E457K, E465Q, N470S, E530K, T538K, D567N, G696S, L812X, E1131M, S1163X, K1249E, F1261L, A1351P, L1411P, L1485P, and combinations thereof. The nucleotide sequence of the Myh9 gene and the protein that encodes it are known in the art, as are the gene and protein sequences from a variety of non-human animals. The human cDNA and protein sequences can be found under GenBank accession no. CR456526.1, Oct. 21, 2008, and those cDNA and amino acid sequences are incorporated herein by reference. The human Myh9 protein sequence is provided under SEQ ID NO:28.

Any one or any combination of the mutations can be detected. The disclosure includes detecting the mutation(s) at the DNA, RNA and protein levels as further described below. The method includes determining homozygosity for the presence or absence of a mutation, as well as for determining hemizygosity for the mutations. The method also comprises determining whether or not the cancer cells exhibit low expression of mysosin-IIA relative to a suitable control. In embodiments, the presence of any one or any combination of the mutations, and/or low expression of mysosin-IIA, aids in a diagnosis that the individual has an aggressive form of cancer. In embodiments, the presence of any one or any combination of the mutations, and/or low expression of mysosin-IIA, aids in the development of a worse prognosis for the individual relative to an individual with cancer that does not have the mutations or the low expression of mysosin-IIA.

The method is suitable for testing samples from any human individual. Thus, in various embodiments, the disclosure provides compositions and methods that can be used for convenient and rapid determination of the presence of the Myh9 mutations in genomic DNA, in Myh9 mRNA, and/or protein in a sample comprising cancer cells.

Any biological sample can be used. In embodiments, the sample is a sample of a tumor, such as a tumor biopsy. In certain approaches, the sample is obtained from the individual and tested directly. In other embodiments, the sample is obtained and subjected to a processing step before being tested for the Myh9 mutations, and/or amount of Myh9 mRNA and/or protein. In some examples, the processing step can be carried out to isolate, and/or purify and/or amplify the Myh9 genomic DNA, mRNA, cDNA, or to isolate the myosin-IIA protein.

Detection of the Myh9 mutations at the nucleic acid level can be performed using any method. The nucleic acids may be detected directly, or they may be manipulated to facilitate detection. The method is amenable to being performed as part of a multiplexed assay, and can be performed using commercially available components adapted to detect the Myh9 nucleic acids. As such, the nucleic acids can be detected using a chip or an array. In various embodiments, a low level of Myh9 mRNA, or the mutations in DNA or RNA, are detected using a polymerase chain reaction (PCR)-based approach. Thus, Myh9 polynucleotides can be amplified enzymatically in vitro. For amplification reactions, primers can be designed which hybridize to the Myh9 gene or its RNA, and used to obtain nucleic acid amplification products (i.e., amplicons). Those skilled in the art will recognize how to design suitable primers and perform amplification and/or hybridization reactions in order to carry out various embodiments of the method of this disclosure. Generally, the sequence of amplified polynucleotides will be determined using any of a number of techniques so that the presence or absence of the mutations can be determined The disclosure includes forming and detecting complexes of synthetic oligonucleotide probes, such as PCR primers, with genomic DNA, RNA, and/or cDNA. The disclosure includes detecting cDNA, RNA, and genomic DNA by testing a synthetically created plurality of amplicons for the presence or absence of the mutations. The method comprises detecting nucleic acids using probes that are fixed to a solid substrate, wherein a complex of the nucleic acid and the probe is detected.

The method in certain embodiments includes Real-Time (RT) PCR, including quantitative real-time (QT-RT or qRT-PCR) PCR analysis, or any other in vitro amplification methods. For amplification reactions, primers can be designed which hybridize to mRNA transcribed from the Myh9 gene, and used to obtain nucleic acid amplification products (i.e., amplicons). Those skilled in the art will recognize how to design suitable RT=PCR primers and perform amplification and/or hybridization reactions in order to carry out various embodiments of the method of the invention. In general, suitable primers are at least 12 bases in length, but primers as short as 8 bases can be used depending on reaction conditions. The primers/probes used for detecting Myh9 gene RNA can comprise modifications, such as being conjugated to one or more detectable labels, such as fluorophores in the form of a reporter dye and/or a quenching moiety for use in reactions such as real time (RT)-PCR, including qRT-PCR, which allow quantitation of DNA amplified from RNA, wherein the quantitation can be performed over time concurrent with the amplification. In one embodiment, the amplification reaction comprises at least one polynucleotide probe specific for Myh9 encoded mRNA, wherein the probe includes one terminal nucleotide modified to include a fluorescent tag, and the other terminal nucleotide modified to comprise a moiety that quenches fluorescence from the fluorescent tag. For instance, for use in RT-PCR, such a probe can be designed so that it binds with specificity to a portion of Myh9 encoded mRNA, or its complement that is between and does not overlap sequences to which two RT-PCR primers hybridize. Using this design, signal from the fluorescent tag will be quenched until the probe is degraded via exonuclease activity of the polymerase during amplification, at which point the fluorescent nucleotide will be separated from the quenching moiety and its signal will be detectable.

It will be recognized by those skilled in the art that while particular sequences of primers are provided herein, other primer sequences can be designed to detect the Myh9 encoded mRNA. In certain embodiments, at least two synthetic oligonucleotide primers are used in an amplification reaction. The primers in different embodiments can be from 8 to 100 nucleotides in length, inclusive, and including all integers there between. The primers are of sufficient length and nucleotide composition to specifically hybridize under stringent conditions to Myh9 encoded mRNA, mRNA, and to cDNA equivalents thereof. In non-limiting examples, a first synthetic primer for use in an amplification reaction comprises or consists of a polynucleotide sequence that is identical to at least 8 contiguous nucleotides in the Myh9 encoded mRNA sequence, and a second primer comprises or consists of a polynucleotide sequence that is complementary to at least 8 contiguous nucleotides in the Myh9 encoded mRNA sequence. Longer primers can tolerate a certain number of mismatched nucleotides that will be apparent to one skilled in the art, and are dictated by such well known parameters as melting temperature and stringency. The primers can be designed such that they do not have complementarity to one another.

In alternative embodiments, mutant myosin-IIA protein can be detected. Detection of the presence or absence of mutant protein can be performed using, for example, any immunological-based detection mechanism that can distinguish mutant from non-mutant protein, including but not necessarily limited to ELISA assays and immunohistochemistry approaches.

In embodiments, a metabolic-based assay, such as an assay for myosin-IIA ATPase activity can be performed and compared to a suitable control to determine whether or not the myosin-IIA in the sample exhibits normal or defective ATPase function.

The determination of the amount of mysosin-IIA expression to ascertain whether its expression is low can be performed at the mRNA and/or protein level using any suitable techniques for quantitating mRNA or protein. In embodiments, the amount of mysosin-IIA protein and/or mRNA can be compared to a reference. The reference can be any suitable reference, examples of which include but are not limited to samples obtained from tumors which have normal mysosin-IIA expression and function, or a standardized curve(s), and/or experimentally designed controls such as known input RNA or protein used to normalize experimental data for qualitative or quantitative determination of the mysosin-IIA expression from the sample for mass, molarity, concentration and the like. The reference level may also be depicted as an area on a graph. In certain embodiments, determining the presence of one or more of the mutations, and/or lower mysosin-IIA expression in a sample is a diagnosis of an aggressive form of cancer, such as a squamous cell carcinoma, or aids in a diagnosis of an aggressive form of a cancer. In embodiments, a determination that the amount of mysosin-IIA is low means the myosin-IIA expression is 5% or less than that of a suitable reference. In embodiments, the reference is a sample of a non-aggressive form of the cancer, or a matched cell type that is non-malignant. In this regard, and as will be more fully appreciated from the examples and figures presented herein, we have determined by univariant Kaplan Meier Survival that low Myh9 expression (bottom 5%) is significantly correlated with reduced survival of HNSCC patients, with a median survival of 13.6 months compared to 28.3 months i.e., FIG. 4D). Likewise, we have observed low myosin-IIa protein expression and even loss of myosin-IIa protein expression in HNSCC and skin SCC (i.e., FIGS. 4B and C). When MYH9 mRNA is analyzed, we observed a distribution of expression (see FIG. 23A), where the lowest 5% corrletates with redced survival but higher mRNA levels did not. In addition, we demonstrate that cells lacking Myh9 or expressing mutant Myh9 are unable to properly respond to DNA damaging agents and consequently cannot activate p53 and p53 target genes, including but not necessarily limited to the pro-apotopic Fas and Bax genes, and the cell senescence gene referred to as p21. Thus, since it is known in the art that the present standard of care for HNSCC patients involves treatment with DNA damaging agents, including but not necessarily limited to radiation or cisplatin-treatment, results presented in this disclosure can be used to predict that Myh9-defective tumor cells will not response to DNA damaging treatments unless a nuclear export inhibitor is used in combination with it, thereby counteracting the effect of mutant or defective myosin-IIa on p53 activation.

In another aspect, the disclosure provides a method for selecting an individual as a candidate for therapy with a nuclear export inhibitor. This aspect involves testing a sample for the Myh9 mutations and/or a low amount of Myh9 expression as described herein, and subsequent to determining the presence of the mutations and/or the low amount of Myh9 expression, designating the individual as a candidate for the therapy with a nuclear export inhibitor. Likewise, the absence of the mutations or a normal level of Myh9 expression indicates the individual is not a candidate for therapy with a nuclear export inhibitor. In certain embodiments, the method involves treating the individual with a nuclear export inhibitor subsequent to detecting one or more of the mutations and/or low Myh9 expression.

In embodiments, a result based on a determination of the presence or absence of the mutations, and/or the amount of the Myh9 expression, can be fixed in a tangible medium of expression, such as a digital file saved on a portable memory device, or on a hard drive. The determination can be communicated to a health care provider for aiding in the diagnosis of a disorder associated with the mutations and/or low expression of the Myh9 gene.

In another aspect the disclosure includes a method for determining whether cancer cells have defective p53 nuclear transport. In embodiments, “defective nuclear transport” means that p53 does not accumulate in the nucleus in response to DNA damage to the same degree that p53 accumulates in the nucleus of a control cell that does not have the mutations in the Myh9 gene.

The method comprises testing cancer cells for a mutation in the Myh9 gene or low expression of mysosin-IIA, wherein the presence of the mutation in the Myh9 gene or low expression of mysosin-IIA determines that the cells have defective p53 nuclear transport.

In embodiments, any of the approaches described herein can be performed in vitro.

In an embodiment, the disclosure includes a method for prophylaxis and/or therapy of a subject who has been diagnosed with, is suspected of having, or is at risk for developing an aggressive form of cancer. Such individuals include those who have cancer or are at risk for recurrence of a cancer, wherein the genome of the cancer cells comprise a mutation that affects the function of mysosin-IIA, and/or the cancer cells exhibit low myosin-IIA expression as further described above. The method comprises administering to the individual a composition comprising an effective amount of a nuclear export inhibitor such that the growth of a tumor comprising the cancer cells is inhibited, and/or such that the survival of the individual is extended, and/or such that the cancer cells are sensitized to chemotherapeutic agents relative to cancer cells that are not exposed to the nuclear export inhibitor, and/or such that the cancer cells are characterized as being capable of having p53 accumulate in the nucleus in response to DNA-induced damage.

In embodiments, the individual to which the nuclear export inhibitor is administered has a cancer of the oral cavity, a skin cancer, a mammary gland cancer, or a squamous cell carcinoma. In embodiments, the squamous cell carcinoma is a head and neck cancer.

It is expected that any nuclear export inhibitor can be used. In embodiments, the nuclear export inhibitor is leptomycinB (LeptB), which is an inhibitor of the Crml nuclear export receptor. Other nuclear export inhibitor can be used, and other export receptors can be inhibited in performing the method of the disclosure. The nuclear export inhibitors can be used in combinations with other chemotherapeutic agents. In embodiments, the other chemotherapeutic agents can comprise MDM2, p53 pathway inhibitors such as Nutlin-3a, protease inhibitors, or combinations thereof.

Administration of a pharmaceutical composition comprising the inhibitor can be performed using any acceptable route and form of delivery. Some non-limiting examples include oral, parenteral, subcutaneous, intraperitoneal, intrapulmonary, topical and intranasal. Parenteral infusions include intramuscular, intravenous, intraarterial, intraperitoneal, and subcutaneous administration. Administration of the compositions can be performed in conjunction with conventional therapies that are intended to treat the particular cancer in question. For example, the composition could be administered prior to, concurrently, or subsequent to conventional anti-cancer therapies. Such therapies can include but are not limited to chemotherapies, surgical interventions, and radiation therapy.

Routes and frequency of administration of pharmaceutical compositions comprising the nuclear export inhibitor, as well as dosage, will vary from individual to individual, and may be readily established using standard techniques, such as the age of the individual, the type and stage of the cancer.

The following examples are presented to illustrate embodiments of the present disclosure. They are not intended to limiting in any manner

### EXAMPLE 1

This example identifies Myh9 as a new tumor suppressor that regulates p53 activation and is often mutated in cancers with poor survival.

Modern genomics is revealing hundreds of genetic alterations associated with cancer. Mining this information for cancer therapies is now predicated on weeding out ‘bystander’ alterations, identifying the ‘driver’ mutations responsible for initiating tumorigenesis and/or metastasis, and elucidating how these mutations alter the fundamental molecular pathways governing tissue growth. Here, we devise and employ a direct in vivo RNAi screening methodology in mice that allows us to simultaneously test candidates whose alterations are associated with head and neck squamous cell carcinomas (HNSCCs) in humans. We identified nine tumor suppressors, seven of which have not been directly linked to tumor development. Our top hit, Myh9, encodes the non-muscle myosin-IIa heavy chain (NMHCIIa). We show that Myh9 functions as a potent tumor suppressor not only in the oral cavity, but also skin and mammary gland. On tumor-susceptible backgrounds, tissue-specific Myh9 RNAi and knockout trigger formation of multiple invasive SCCs and even distant lung metastasis. Surprisingly, myosin-IIa's function is manifested not only in conventional actin-related processes, but also in regulating DNA damage-induced, post-transcriptional p53 activation. Moreover, ˜20% of human HNSCCs have lost myosin-IIa protein expression, ˜5% harbor evolutionarily conserved domain-specific MYH9 mutations, and clinically, low MYH9 expression in HNSCCs correlates with poor survival. These findings establish MYH9 as a major SCC suppressor with prognostic and therapeutic relevance, and also highlight the utility of direct in vivo RNAi to integrate cancer genomics and mouse modeling to rapidly discover and validate potent but low penetrance cancer driver mutations.

To functionally test putative ‘driver mutations’, researchers have used RNA interference (RNAi) followed by allografting of transduced cultured cancer cells. However, orthotopic transplantations necessitate immunocompromised animals and generate wound-responses, which can confound physiological relevance. To circumvent these caveats, we used non-invasive, ultrasound-guided in utero lentiviral-mediated delivery of RNAi, which selectively transduces single-layered surface ectoderm of living E9.5 mouse embryos (FIG. 5A). When an H2B-RFP transgene is inserted into the vector, stable integration/RNAi expression can be monitored by epifluorescence, which is restricted to adult tissues derived from embryonic ectoderm, including skin, oral cavity and mammary gland epithelia. This approach was recently used to identify regulators of oncogenic H-Ras^{G12V}-induced growth in embryos.

To screen genetic/epigenetic alterations in SCCs for tumor-suppressor activities, we modified this strategy for adult mice. We first showed that adult mice transduced at E9.5 with Brcal but not control shRNAs recapitulate the Brcal-knockout phenotype and develop spontaneous skin and oral SCCs with long latency (FIG. 5, B to E). To accelerate tumor growth, we tested our hairpins in K14-Cre;TGFβ-ReceptorII^{floxed/floxed} mice (epithelial-specific, conditional TβRII-knockout), which lose TGFβ signaling in epidermis, oral, anogenital and mammary epithelia and display enhanced SCC susceptibility. Indeed, on this TβRII-cKO background, Brcal-knockdown generated SCCs with increased frequency and <4× latency (FIG. 5B). Having validated our sensitized approach, we devised a pooled shRNA format to carry out our in vivo screen to functionally distinguish driver and bystander mutations and dissect the physiological relevance of epigenetic changes in gene expression that occur in the development of SCC tumor-initiating (stem) cells (FIG. 1A).

We selected 1763 shRNA lentiviruses that targeted 347 mouse genes (˜5 shRNAs/gene) which either a) had human orthologs carrying recurring HNSCC somatic mutations, or b) were deregulated ≧2× in tumor-initiating stem cells purified from TβRII-cKO SCCs, whose cancers were initiated by oncogenic HRas-inducing carcinogens (Table 1). We also included positive (Brcal-shRNA#560) and negative (scramble non-targeting shRNA) controls. We titered our pool such that ˜15-20% of the ˜450,000 surface ectoderm progenitors were infected (FIG. 6A). Based upon library size, ≧20 cells/embryo should be transduced with each shRNA, which if inconsequential should expand clonally 40× by adulthood. To control for coverage, we infected E9.5 epidermis, isolated E12.5 genomic DNA and verified by Illumina sequencing that shRNA representations correlated nicely with their individual abundance within our initial pool (FIG. 6B).

To ensure a coverage of >500 individual clones/shRNA, we infected 74 genotypically matched TβRII-cKO or TβRII^{fl/fl}-control embryos with our pooled (or scrambled-shRNA) lentiviruses and monitored pups into adulthood. As expected, ˜5% of TβRII-cKO mice developed SCCs confined to anogenital epithelia. Scramble-shRNA expression did not affect these statistics nor did transduction with a “control pool” of 1000 random shRNAs.

In striking contrast, two otherwise wild-type mice transduced with our candidate tumor-suppressor shRNA pool developed skin tumors and all 28 transduced TβRII-cKO mice developed lesions within skin, oral cavity and/or mucocutaneous junctions at eyelids (FIG. 1B and FIG. 7A). All TβRII-cKO and other control animals remained tumor-free at these sites. These findings underscored the efficacy of our approach and documented the enrichment of our test-shRNA library for SCC tumor suppressors.

87 lesions were chosen for further analyses. Most displayed histopathological features of SCCs with varying degrees of differentiation and local invasion; a few were squamous papillomas or epidermal hyperplastic lesions, with one benign basal cell tumor (FIG. 7 to 9). Deep sequencing revealed that most lesions harbored one or two transduced shRNAs that were highly enriched relative to initial pool representation and to healthy skin surrounding the tumor; gratifyingly this included Brcal-shRNA#560, our positive-control (FIG. 1C). Nine candidate tumor suppressors were identified that displayed highly enriched multiple independent shRNAs in ≧3 tumors (FIG. 1D).

Strikingly, 40% of tumors were enriched for shRNAs against Myh9, encoding non-muscle myosin-IIa heavy chain (NMHCIIa). These included nearly all tumors emerging by 4 months of age. Importantly, four Myh9-shRNAs in the library were enriched in different tumors of multiple mice (example in FIG. 1 C). Knockdown efficiency of our five Myh9-shRNAs correlated strongly with multiplicity/aggressiveness of tumor growth (FIG. 2A). Tested individually in vivo, the three top Myh9-shRNAs markedly reduced myosin-IIa protein (FIG. 2B and FIG. 10A).

Myh9-knockdown animals showed an ‘open eye at birth’ phenotype. From postnatal day 8 onward, hair coats were visibly sparse (FIGS. 10, B and C). Histology and immunofluorescence showed normal epidermal differentiation, without major changes in either proliferation or apoptosis (FIGS. 11, A and B). Mosaic transductions recapitulated these findings. As Myh9 shRNA-transduced TβRII-cKO mice aged, sparse areas of epidermal thickening appeared, accompanied by expanded immunolabelings for basal keratin K14, and for K6, a suprabasal marker associated with hyperproliferative epidermal disorders (FIGS. 11, C and D).

TβRII-cKO mice transduced with Myh9-shRNA #507, #504 or #503 also developed multiple, highly proliferative and poorly differentiated skin SCCs and HNSCCs with 3-7 month median latencies (FIG. 2C). Tumors were myosin-IIa-deficient, displayed hallmarks of human SCCs, and invaded subcutaneous fat, underlying muscle and salivary glands (FIG. 12, A to G). They colonized draining lymph nodes (FIG. 12F) and even formed distant lung metastases (FIG. 2D). In location, morphology and invasiveness, they differed from the spontaneous anogenital TβRII-cKO tumors that formed at the interface between colonic and squamous epithelia. Finally, ˜25% of TβRII^{fl/fl} (no-Cre) mice transduced with Myh9-shRNAs but not scrambled-shRNAs developed skin SCCs after 1 year, indicating that Myh9 loss alone is sufficient to promote spontaneous tumor development.

To further define MYH9 as an SCC tumor suppressor, we crossed Myh9^{fl/fl} mice to our epithelial-specific K14-Cre and tamoxifen-regulated K14CreER deleter strains. Embryologically, Myh9-cKO mice recapitulated open eye at birth and hair phenotypes (FIG. 13, A and B). In adult mice, inducible deletion of even one Myh9 allele concomitantly with TβRII ablation resulted in multiple invasive SCCs on the back, ears and anal region (FIG. 2E FIG. 13, C to E). Littermate controls remained tumor-free during this time.

Our knockout/knockdown strategies targeted not only skin and oral epithelium but also mammary epithelium. Myh9-shRNA transduced wild-type animals underwent seemingly normal mammary gland formation and/or branching morphogenesis (FIG. 14, A to D). By contrast, TβRII-cKO mice transduced with Myh9-shRNAs frequently displayed multiple mammary lesions by ˜10-12 weeks of age (FIG. 14D). Thus, amidst glands positive for luminal (K8/K18) and myoepithelial (K5/K14) markers, K5/K14, K6 and K10-positive lesions were seen that resembled SCCs. They were H2BRFP-positive and stained poorly for myosin-IIa, reflective of their expression of Myh9-shRNA (FIG. 14, D to G). Their early occurrence suggested that they were primary breast tumors, rather than metastatic lesions from primary skin-SCCs or HNSCCs. Although abrogation of TGFβ signaling sensitizes mammary epithelium to SCC formation, these tumors were not observed with scrambled-shRNAs, underscoring their added dependency on Myh9-knockown.

The pronounced invasion and distant metastases was linked to Myh9-knockdown. Indeed, epithelial outgrowth from skin explants was markedly enhanced when TβRII-cKO embryos were transduced with Myh9 but not control shRNAs (FIG. 15). Similar results were obtained in vitro with scratch-wound (FIG. 16A) and trans-well migration assays (FIG. 2F). This increase was independent of TGFβ signaling status but as substantial as that seen with TβRII ablation. Moreover, reducing Myh9 levels had profound effects on cells challenged to invade and migrate through a Matrigel-coated filter (FIG. 2G).

Our results thus far were consistent with the well-established role for actin-myosin networks in regulating cellular movements. More puzzling was our discovery that Myh9-knockdown showed no tumorigenic effects in mice whose epithelium carried a Trp53 gain-of-function mutation analogous to that found in human HNSCCs. By contrast, under conditions favoring HRas mutations, Myh9-shRNAs greatly accelerated the latency, multiplicity and SCC conversion rate, analogous to our findings with TβRII-ablation (FIG. 17, A to E). This context-dependency raised the possibility that myosin-IIa deficiency and Trp53 mutations may be functionally redundant.

To test for an epistatic interaction of these two pathways, we treated primary keratinocytes with doxorubicin, which introduces double-stand DNA breaks, thereby triggering the DNA damage response (DDR) pathway. In control keratinocytes, this led to p53 activation (FIG. 3A). Notably, however, Myh9 suppression with multiple shRNAs resulted in significantly delayed and less-sustained p53 activity in doxorubicin-treated cultures (FIG. 3A and FIG. 18A). This was also true for Myh9fl/fl keratinocytes transduced in vitro with lentiviral Cre compared to empty control-lentivirus, as well as epidermis of γ-irradiated Myh9-cKO and Myh9-knockdown mice (FIG. 3, B to C and FIG. 18, B to D). Moreover, relative to controls, Myh9-deficient keratinocytes failed to induce p53-responsive genes such as p21, Fas, Bax, Mdm2 and 14-3-3σ (FIG. 3A, C, D and FIG. 18, A to B). Importantly, these effects were specific to the p53 pathway, since control and Myh9-knockdown keratinocytes responded equally well to other stimuli such as EGF (FIG. 19A).

The suppressive effects of myosin-IIa deficiency on p53 activation were also observed with primary mammary epithelial cultures (FIG. 20A). Moreover, these effects were not dependent upon TGFβ signaling, and conversely, TβRII-ablation did not impair the ability of doxorubicin to induce p53 (FIG. 20, B to D). Additionally, such effects were not observed with shRNAs against the other non-muscle myosin-II family members, Myh10 (myosin-IIb) and Myh14 (myosin-IIc) (FIG. 20, E to F).

Together, our findings indicated that the effects on the DDR/p53 pathway are not simply a general phenomenon of SCC tumorigenesis, but rather a specific consequence of myosin-IIa deficiency. Probing deeper, we discovered that the myosin-II kinase inhibitor, blebbistatin, phenocopied Myh9 loss of function effects on DDR-induced p53 activation (FIG. 3E and FIG. 21A). Consistent with a role for myosin-IIa's ATPase function, inhibition of Rho-kinase (Rock), an upstream regulator of myosin-II's ATPase activity, similarly dampened the DNA damage-induced p53 response (FIG. 3E and FIG. 21B). Surprisingly, however, latrunculin-mediated inhibition of F-actin polymerization did not display these effects, raising the tantalizing possibility that these effects may be independent of myosin-IIa's role in the actomyosin cytoskeleton (FIG. 3E and FIG. 21B).

The initial steps of the DDR response appeared to be unperturbed, as judged by stress-induced phosphorylation of the histone variant MAX and activation of DNA checkpoint kinases Chk1 and Chk2 (FIG. 21B). Additionally, Myh9-ablation did not affect Trp53 gene expression as Trp53 mRNA levels were normal (FIG. 2D). However, in the presence of proteasome inhibitor MG132, p53 protein levels were comparably induced in both Myh9-knockdown and control keratinocytes (FIG. 21C).

Seeking how myosin-IIa deficiency might affect p53 stability, we first discovered that p53's nuclear accumulation following DNA damage did not occur when myosin-II ATPase was inhibited (FIG. 3F). We next learned that when DDR-induced myosin-IIa-deficient keratinocytes were treated with leptomycinB (LeptB), an inhibitor of the Crml nuclear export receptor, nuclear p53 accumulation as well as transactivation of the p53 target genes such as CDKN2 (p21) were restored to normal levels (FIG. 3F and FIG. 21D). This shows that the p53 pathway can be induced in response to DNA damage even when myosin-IIa is defective but it fails to do so owing to a specific inability to remain in the nucleus.

Our initial screen included MYH9 because of its mutations in exome-sequenced HNSCCs. Given the possible clinical relevance of LeptB as a means to overcome p53 effects in myosin-IIa-defective tumors, we first confirmed that p53 activation is similarly compromised in MYH9-deficient primary human keratinocytes (FIG. 4A). Moreover, upon surveying myosin-IIa's status in >350 human skin, head and neck SCCs and control tissues, we found that in contrast to normal and hyperplastic skin, which consistently displayed strong immunolabeling, 24% of skin SCCs and 31% of HNSCCs showed weak or no immunolabeling (FIGS. 4, B and C and FIG. 22, A to C). K14 immunohistochemistry internally controlled for tissue quality. Interestingly, myosin-IIa was diminished in a number of early stage, i.e. grade I, SCCs, indicating that its loss may constitute an early event in tumor progression (FIG. 22D). Additionally, when skin SCCs were analyzed according to TβRII and P-Smad2 status, a substantial fraction (˜83%) of myosinIIa-negative tumors showed signs of concomitant loss of TGFβ signaling (FIG. 22E).

Finally, we exploited The Cancer Genome Atlas (TCGA) in order to determine whether MYH9-mRNA expression correlates with HNSCC patient survival. Remarkably, univariate analysis revealed a significant correlation between the lowest MYH9-mRNA expression (bottom 5%) and reduced time to death in HNSCC patients (30.0 vs. 13.6 months; n=166 patients; p=0.0044, log rank test; for detailed analysis, visit: http://bit.ly/13xxPuh) (FIG. 4D). By contrast, even though some patients showed either increased MYH9-mRNA levels or MYH9 amplifications, Kaplan-Maier Analysis revealed no survival advantage or disadvantage in this cohort (FIG. 23, A to C).

The TCGA database contained 13 missense or truncating MYH9 mutations in their cohort of 302 sequenced HNSCCs (FIG. 4E) in addition to others that were previously identified. Notably, patients harboring these mutations or reduced MYH9 expression associate with significantly shorter survival than other HNSCCs (15.2 vs. 26.4 month; n=166 patients p=0.0156, log rank test) (FIGS. 25 A and B).

Computational analyses of evolutionary conservation patterns yields a functional impact score (FIS), which predicts the putative impact of an amino acid residue change on a protein and assigns a probability that such a mutation will result in functional consequences at the level of the organism. Interestingly, all 15/16 of these MYH9 mutations thus far found in human HNSCCs had a high or medium FIS score, indicative of positive selection for these mutations (FIG. 4E and FIGS. 24, A and B). Indeed, statistical analysis of the TCGA data set revealed that high-scoring functional MYH9 mutations are significantly overrepresented in HNSCCs (p=0.000026), but also in a number of other cancers, including lung SCCs and breast cancer (Table 2 and 3; FIG. 25C).

These cancer-associated MYH9 mutations were not randomly distributed across the gene, as would be expected for mutations, which accumulate randomly over time. Rather, they showed a clear signature of selection, with a preferential clustering to the Myosin Head domain and especially the highly-conserved ATPase SwitchII region (FIG. 4E; p=0.0015). Notably, a point mutation in this region of Dictyostelium myosin II compromises ATPase activity—in fact, mutations of the exact same conserved amino acid (A454) are found in human HNSCCs (FIG. 4E and FIG. 24B). Site-directed point mutagenesis of human MYH9 further corroborated these bioinformatic predictions. Thus, while the MYH9-E475K and also MYH9-F1261L mutants retained its ability to localize to stress fibers, it exerted dominant negative effects on p53 activation (FIG. 4E and FIGS. 26, A and B).

Based upon this predicted functionality of mutations, MYH9 ranked 16th among all 15,086 genes altered in HNSCCs (p-value 0.000026) (Table 2). Based upon another algorithm for mutation calling (MutSig), Myh9 ranks 49^{th} (Table 4). Additionally, ˜15% of all HNSCCs in the TCGA dataset show hemizygous loss of one MYH9 allele. This facet is particularly intriguing given our functional analyses showing that Myh9 heterozygosity predisposes mouse epithelia to SCC formation. Hemizygous MYH9 loss is also common in other epithelial cancers—1076 out of 3081 cases within the entire TCGA dataset show monoallelic loss of MYH9 (FIG. 4F and Table 3 and 5). Although homozygous deletion, amplifications or gains exist, they are not significantly overrepresented in these cancers, nor would severe alterations be expected given the essential role for myosin-IIa in actomyosin networks.

MYH9 had not previously been exposed functionally as a tumor suppressor, and hence it was remarkable that it not only surfaced as our top hit but in addition, its loss led to spontaneous, highly invasive and metastatic SCCs. The inverse relation was particularly puzzling, as dominant active Rho kinase and/or extracellular matrix (ECM) stiffness contribute and even promote transformation in some cell lines and animal models. That said, primary human cancers cells are considerably more pliable, and indeed our results indicate that a reduction in actin-myosin can confers transforming potential. The most striking link between myosin-IIa and cancer, however, seems to be independent of the conventional role for myosin-IIa in actomyosin dynamics. Given our new findings that myosin-IIa profoundly affects p53 activation, we view myosin-IIa as a multifaceted tumor suppressor at the crossroads between migration, invasion and survival.

The following provides a description of the materials and methods used to obtain the results presented and described herein.

Materials and Methods. Mice and lentiviral transductions: TβRII foxed mice were crossed to K14-Cre and/or Rosa26YFPlox/stop/lox mice and or K14-CreER mice. Myh9 floxed mice were purchased form EMMA (EM:02572). CD1 mice were from Charles River laboratories. Large-scale production and concentration of lentivirus (6−10⁹ cfu/ml) as well as ultrasound-guided lentiviral injection were performed as previously described. As controls for knock-down mice, littermates were infected with a non-targeting scrambled-shRNA, which activates the endogenous microRNA processing pathway but is not known to target any gene. Myh9fl/fl K14CreER mice were injected i.p. with 2 mg tamoxifen (20 mg/ml stock solution in corn oil) for 5 consecutive days at 6-8 weeks of age. DMBA/TPA treatment was performed as previously described. Briefly, 7-8 week old CD1 mice in second telogen were shaved and treated with 400 nmol DMBA in 100 ul aceton one week later. Thereafter, mice were treated with 17 nM TPA in 100 ul aceton wice weekly for 20 weeks. All animals were maintained in an AAALAC-approved animal facility and procedures were performed with protocols approved by IACUC and in accordance with the National Institutes of Health.

Constructs and RNAi: shRNA constructs for the shRNA pool were obtained from The Broad Institute's Mission TRC-1 mouse library. We tested and used especially the following shRNAs targeting Brcal and Myh9:

Brca1 #560 TRCN0000042560
(SEQ ID NO: 1)
5′-CCCATCATACTTTAATGTGTA-3′
Myh9 #503 TRCN0000071503
(SEQ ID NO: 2)
5′-GCCCTGGAACTGTGTTTAGAA-3′
Myh9 #504 TRCN0000071504
(SEQ ID NO: 3)
5′-CGGTAAATTCATTCGTATCAA-3′
Myh9 #505 TRCN0000071505
(SEQ ID NO: 4)
5′-GCACACATTGACACAGCCAAT-3′
Myh9 #506 TRCN0000071506
(SEQ ID NO: 5)
5′-GCCATACAACAAATACCGCTT-3′
Myh9 #507 TRCN0000071507
(SEQ ID NO: 6)
5′-GCGATACTACTCAGGGCTTAT-3′

The scrambled shRNA 5′-CAACAAGATGAAGAGCACCAA-3′ (SEQ ID NO:7) was used for the control. These hairpin sequences were cloned from the library vectors into pLKO-H2B-RFP vector. All other hairpins were obtained from the TRC library and are listed in Table 1.

Tumor free survival: Control and TβRII-cKO animals were transduced at E9.5 with low-titer shRNA pool targeting orthologs of putative HNSCC genes, including Brcal or Myh9. Scrambled shRNA was used as control. Transductions and knockdowns were confirmed by real-time PCR of mRNAs isolated from newborn skin epidermis or by fluorescence microscopy of a lentiviral reporter fluor, H2B-RFP or H2B-GFP. Animals were assessed biweekly for signs of tumorigenesis, and were considered positive if lesions grew to be larger than 2 mm in diameter.

Deep Sequencing: Sample preparation, preamplification and sequence processing Epidermal and tumor cells were subjected to genomic DNA isolation with the DNeasy Blood & Tissue Kit (Qiagen), and each sample was analyzed for target transduction using real-time PCR. 6 μggenomic DNA of each tumor was used as template in a pre-amplification reaction with 25 cycles and Phusion High-Fidelity DNA Polymerase (NEB). PCR products were run on a 2% agarose gel, and a clean ˜200 bp band was isolated using QIAquick Gel Extraction Kit as recommended by the manufacturer (Qiagen). Final samples were then sent for Illumina HiSeq 2000 sequencing. Illumina reads were trimmed to the 21 nt hairpin sequence using the FASTX-Toolkit and aligned to the TRC 2.x library with BWA (v 0.6.2)44 using a maximum edit distance of 3. Hits were ranked based on (a) numbers of shRNAs that targeted the gene and scored positively in the screen, with 2 out of 5 shRNAs being considered meaningful; and (b) numbers of tumors enriched for a specific shRNA.

Immunofluorescence staining The following primary antibodies were used for immunofluorescence: chicken anti-GFP (1:2000; Abcam); guinea-pig anti-K5 (1:500; E. Fuchs); rat anti-K14 (1:500; E. Fuchs); rabbit anti-K6 (1:500; E. Fuchs); rabbit anti-K18 (1:500; E. Fuchs); rat anti-CD104 β4-integrin (346-11A, 1:300; BD); rabbit anti loricin (1:500; E. Fuchs); rabbit anti-Caspase 3 (AF835, 1:1000; R&D), rabbit anti-K10 (PRB-159P, 1:1000; Covance); rabbit anti-Myh9 (HPA001644, 1:500 Sigma); rabbit anti-SMA (ab5694 1:300; Abcam) and rabit anti-p53 (NCL-p53-CMSp, 1:300; Leica). Secondary antibodies were conjugated to Alexa-488, 546, or 647 (1:1000, Life Technologies). Cells and tissues were processed as previously reported, and mounted in Vectashield HardSet mounting medium with DAPI (Life Technologies). Confocal images were captured by a scanning laser confocal microscope (LSM510 and LSM780; Carl Zeiss, Inc.) using Plan-Apochromat 20×/0.8 oil and C Apochromat 40×/1.2 water lenses. Images were processed using ImageJ and Adobe Photoshop CS3. For quantifications of nuclear p53, images were captured using an inverted Zeiss LSM 780 laser scanning microscope, powered by Zen software. Quantitative image analysis was performed using ImageJ software. To quantify p53 nuclear staining, the following formula was used: CTCF (corrected total cell fluorescence)=whole nucleus signal−(mean background signal (measured in the suprabasal layer)×area of the nucleus measured).

Immunohistochemistry and histological analyses of mouse and human Tumors: Immunohistochemistry was performed as previously described. Briefly, 5-μm sections were cut, stained with H&E or processed for immunohistochemistry/immunofluorescence microscopy. Whole-mount staining of mammary glands was performed as described. For immunoperoxidase staining, paraffin-embedded sections were dehydrated and antigenic epitopes exposed using a 10-mM citrate buffer (pH 6.0) in a pressure cooker. Sections were incubated with the following primary antibodies at 4° C. overnight: rabbit anti-K14 (1:500; E. Fuchs) and rabbit anti-Myh9 (HPA001644, 1:500 Sigma). Primary antibody staining was visualized using peroxidase-conjugated anti-rabbit IgG followed by the DAB substrate kit for peroxidase visualization of secondary antibodies (Vector Laboratories). The following human tissue microarray comprising 48 healthy human skin samples, 30 hyperplastic skin lesions and 206 human skin SCCs as well as from 156 HNSCCs were obtained from US Biomax, Rockeville, Md.: SK244a, SK241, SK242, SK801, SK802, SK2081, SK801b and HN803a, HN811a, HN483.

Western Blot analysis:₋Protein blotting was carried out using standard protocols. Briefly, total cell lysates were prepared using RIPA (20 mM Tris-HCl (pH 8.0), 150 mM NaCl 1 mM EDTA, 1mM EGTA, 1% Triton X-100, 0.5% Deoxycorate, 0.1% SDS, 25 mM β-glycerophosphate, 10 mM NaF, 1 mM Na3VO4) supplemented with protease inhibitors (Complete mini, Roche). Blots were blocked with 5% BSA in 1×TBS 0.1% Tween-20 (TBST) for 1 h and incubated with the primary antibody overnight at 4° C. (diluted in TBST according to the manufacturer's protocol). Primary antibodies were reactive to rabbit anti-Myh9 (1:500, HPA001644, Sigma); phosphorylated (P) Erk1/2 (1:1000, #9101, Cell Signaling), Erk1/2 (1:1000, #9102, Cell Signaling), mouse anti-p53 (1:500, #2524, Cell Signaling), mouse anti-p21(F5) (1:500; sc-6246, Santa Cruz), mouse anti-GAPDH (ab8245, 1:5000; Abcam), mouse anti-Chk2 (1:500, #611570, BD); rabbit anti-P-Chk1 (1:500, #12302P, Cell Signaling); mouse anti-Chk1 (1:1000, 2360S, Cell Signaling); rabbit anti-pSmad2 (Ser465/467) (1:1000, Cell Signaling) and mouse anti-Smad2/3 (610843, 1/500; BD). Blots were washed three times in TBST for 30 min, incubated with HRP-conjugated secondary antibodies (1:2,000; Promega) for lh at room temperature, washed 3 times in TBST for 30 min and visualized using enhanced chemiluminescence (ECL).

p53/DNA damage responses:_For measurement of DNA damage response and p53 activation primary mouse keratinocytes cells were seeded at a cell density of 100,000 cells per well in a 6-well plate and allowed to grow for 24 h at 3% O2 till importantly 100% confluency. Cells were then treated with doxorubicin (1 mM) as previously reported. For experiments using blebbistatin, cells were pretreated with blebbistatin (4 μM final concentration, Sigma B0560) 30 min prior to doxorubicin treatment. The Rock inhibitor Y27632 was used at 10 μM (Sigma Y0503), LatrunculinA was used at 2 μM (Sigma L5163), LeptomycinB was used at 20 nM (Sigma #9676) and the proteasome inhibitor MG132 was used at 3 μM (Sigma M7449).

mRNA quantifications: Newborn mouse epidermal keratinocytes were cultured in 0.05 mM Ca++ E-media supplemented with 15% serum. For lentiviral infections, cells were plated in 6-well dishes at 200,000 cells/well and incubated with lentivirus in the presence of polybrene (100 mg/ml) overnight. After 2 days, infected cells were positively selected with puromycin (1 mg/ml) for 3 days, and then processed for mRNA analysis. cDNAs were generated from 1 μg of total RNA using the SuperScript Vilo cDNA synthesis kit (Life Technologies). Real-Time PCR was performed using the 7900HT Fast Real-Time PCR System (Applied Biosystems) and gene-specific and Ppib as well as Hprt1 control primers as well as the following primers for p53 target genes:

p21 (Cdkn1a) fwd primer
(SEQ ID NO: 8)
5′-GTGGCCTTGTCGCTGTCTT-3′
p21 (Cdkn1a) rev primer
(SEQ ID NO: 9)
5′-GCGCTTGGAGTGATAGAAATCTG-3′
Fas fwd primer
(SEQ ID NO: 10)
5′-CTGCGATGAAGAGCATGGTTT-3′
Fas rev primer
(SEQ ID NO: 11)
5′-CCATAGGCGATTTCTGGGAC-3′
Bax forward primer
(SEQ ID NO: 12)
5′-ATGCGTCCACCAAGAAGCTGA-3 ′
Bax reverse primer
(SEQ ID NO: 13)
5′-AGCAATCATCCTCTGCAGCTCC-3′
Mdm2 forward primer
(SEQ ID NO: 14)
5′-TTCGGCCTTCTCCTCGCTGTCGTC-3′
Mdm2 reverse primer
(SEQ ID NO: 15)
5′-TGGCGTAAGTGAGCATTCTGGTGA-3′
Bax forward primer
(SEQ ID NO: 16)
5′-TGTGTGCGACACTGTGCTC-3′
Bax reverse primer
(SEQ ID NO: 17)
5′-TCGGCTAGGTAGCGGTAGTAG-3′
Hprt1 for primer
(SEQ ID NO: 18)
GATCAGTCAACGGGGGACATAAA
Hprt1 rev primer
(SEQ ID NO: 19)
CTTGCGCTCATCTTAGGCTTTGT
Ppib for primer
(SEQ ID NO: 20)
GTGAGCGCTTCCCAGATGAGA
Ppib rev primer
(SEQ ID NO: 21)
TGCCGGAGTCGACAATGATG

Explant and Migration/Invasion Assay:_Explant outgrowth migration assays were performed as described previously. Briefly, explants were cut using a 3-mm dermal biopsy punch (Miltex), placed on fibronectin-coated 35-mm, glass-bottomed plates (MatTek), and submerged in E-media containing 0.6 mM Ca++. Explant outgrowth was monitored daily.

Transwell migration assays were performed on 24-well plates. The underside of each Boyden chamber well was coated with 10 μg/ml fibronectin and placed atop fibroblast-conditioned E-media containing 0.05 mM Ca++. A total of 50,000 keratinocytes/well were plated in 100 μl E-medium containing 0.05 mM Ca++. Eight hours later, cells were washed off the top membrane and fixed on the bottom membrane. Cells were stained using H&E and counted under the microscope. Similarly, invasion assays were performed in precoated Matrigel invasion chamber (BD Biosciences).

Analysis of human HNSCC patient data: We analyzed the publicly available data sets of the The Cancer Genome Atlas (TCGA: http://cancergenome.nih.gov). The cBioPortal for Cancer Genomics developed and maintained by the Computational Biology Center at Memorial Sloan-Kettering Cancer Center was used to mine the publicly available TCGA dataset on HNSCC. To re-trace the exact Kaplan-Meyer analysis please visit http://bit.ly/13xxPuh for the analysis of HNSCC patients stratified by the lowest (<5th percentile) MYH9 expression versus the rest (≧5th percentile) and http://bit.ly/YK0dYy for the analysis of HNSCC patients stratified by the lowest (<5th percentile) MYH9 expression or/and harboring MYH9 mutations versus the rest (≧5th percentile).

Statistical Analysis: All data were collected from experiments performed at least three times, and expressed as mean ± standard deviation (s.d.) or standard error of the mean (s.e.m.). Differences between groups were assayed using two-tailed student t-test and Prism 5 (GraphPad Software). Differences were considered significant if P<0.05. Data were analyzed and statistics performed (unpaired two-tailed Student's t-test) in Prism5 (GraphPad). Significant differences between two groups are noted by asterisks or p-values.

TABLE 1
Genes and shRNA construct included in the shRNA library. The Clone
column provides the name of the construct as given in the Public TRC
Portal of The RNAi consortium. The construct name is indexed in the
Public TRC Portal with NCBI accession numbers and other information
about the shRNA constructs. All of the information and the
constructs are publicly available.

| Gene # | Construct # | Clone | gene |
|---|---|---|---|
| 1 | 1 | TRCN0000179370 | 1500026B10Rik |
| | 2 | TRCN0000179624 | 1500026B10Rik |
| | 3 | TRCN0000179770 | 1500026B10Rik |
| | 4 | TRCN0000184447 | 1500026B10Rik |
| | 5 | TRCN0000184474 | 1500026B10Rik |
| 2 | 6 | TRCN0000126479 | 2010107G23Rik |
| | 7 | TRCN0000126480 | 2010107G23Rik |
| | 8 | TRCN0000126481 | 2010107G23Rik |
| | 9 | TRCN0000126482 | 2010107G23Rik |
| | 10 | TRCN0000126483 | 2010107G23Rik |
| 3 | 11 | TRCN0000176661 | 2310057J16Rik |
| | 12 | TRCN0000177579 | 2310057J16Rik |
| | 13 | TRCN0000182145 | 2310057J16Rik |
| | 14 | TRCN0000182145 | 2310057J16Rik |
| | 15 | TRCN0000182753 | 2310057J16Rik |
| 4 | 16 | TRCN0000113435 | Abca6 |
| | 17 | TRCN0000113436 | Abca6 |
| | 18 | TRCN0000113437 | Abca6 |
| | 19 | TRCN0000113438 | Abca6 |
| | 20 | TRCN0000113439 | Abca6 |
| 5 | 21 | TRCN0000113440 | Abca9 |
| | 22 | TRCN0000113442 | Abca9 |
| | 23 | TRCN0000113443 | Abca9 |
| | 24 | TRCN0000113444 | Abca9 |
| 6 | 25 | TRCN0000105260 | Abcd4 |
| | 26 | TRCN0000105261 | Abcd4 |
| | 27 | TRCN0000105262 | Abcd4 |
| | 28 | TRCN0000105263 | Abcd4 |
| | 29 | TRCN0000105264 | Abcd4 |
| 7 | 30 | TRCN0000087968 | Abi3 |
| | 31 | TRCN0000087969 | Abi3 |
| | 32 | TRCN0000087970 | Abi3 |
| | 33 | TRCN0000087971 | Abi3 |
| | 34 | TRCN0000087972 | Abi3 |
| 8 | 35 | TRCN0000022604 | Acvr1c |
| | 36 | TRCN0000022605 | Acvr1c |
| | 37 | TRCN0000022606 | Acvr1c |
| | 38 | TRCN0000022607 | Acvr1c |
| | 39 | TRCN0000022608 | Acvr1c |
| 9 | 40 | TRCN0000032274 | Adamts12 |
| | 41 | TRCN0000032275 | Adamts12 |
| | 42 | TRCN0000032276 | Adamts12 |
| | 43 | TRCN0000032277 | Adamts12 |
| | 44 | TRCN0000032278 | Adamts12 |
| 10 | 45 | TRCN0000114956 | Adcy8 |
| | 46 | TRCN0000114957 | Adcy8 |
| | 47 | TRCN0000114958 | Adcy8 |
| | 48 | TRCN0000114959 | Adcy8 |
| | 49 | TRCN0000114960 | Adcy8 |
| 11 | 50 | TRCN0000086608 | Aff3 |
| | 51 | TRCN0000086609 | Aff3 |
| | 52 | TRCN0000086610 | Aff3 |
| | 53 | TRCN0000086612 | Aff3 |
| 12 | 54 | TRCN0000071348 | Ahctf1 |
| | 55 | TRCN0000071349 | Ahctf1 |
| | 56 | TRCN0000071350 | Ahctf1 |
| | 57 | TRCN0000071351 | Ahctf1 |
| | 58 | TRCN0000071352 | Ahctf1 |
| 13 | 59 | TRCN0000101420 | Allc |
| | 60 | TRCN0000101421 | Allc |
| | 61 | TRCN0000101422 | Allc |
| | 62 | TRCN0000101423 | Allc |
| | 63 | TRCN0000101424 | Allc |
| 14 | 64 | TRCN0000022614 | Amhr2 |
| | 65 | TRCN0000022615 | Amhr2 |
| | 66 | TRCN0000022616 | Amhr2 |
| | 67 | TRCN0000022617 | Amhr2 |
| | 68 | TRCN0000022618 | Amhr2 |
| 15 | 69 | TRCN0000090053 | Ank3 |
| | 70 | TRCN0000090054 | Ank3 |
| | 71 | TRCN0000090055 | Ank3 |
| | 72 | TRCN0000090056 | Ank3 |
| | 73 | TRCN0000090057 | Ank3 |
| 16 | 74 | TRCN0000090263 | Anln |
| | 75 | TRCN0000090264 | Anln |
| | 76 | TRCN0000090265 | Anln |
| | 77 | TRCN0000090266 | Anln |
| 17 | 78 | TRCN0000110725 | Anxa3 |
| | 79 | TRCN0000110726 | Anxa3 |
| | 80 | TRCN0000110727 | Anxa3 |
| | 81 | TRCN0000110728 | Anxa3 |
| | 82 | TRCN0000110729 | Anxa3 |
| 18 | 83 | TRCN0000012278 | Apaf1 |
| | 84 | TRCN0000012280 | Apaf1 |
| | 85 | TRCN0000012281 | Apaf1 |
| | 86 | TRCN0000012282 | Apaf1 |
| 19 | 87 | TRCN0000026148 | Ar |
| | 88 | TRCN0000026177 | Ar |
| | 89 | TRCN0000026189 | Ar |
| | 90 | TRCN0000026195 | Ar |
| | 91 | TRCN0000026211 | Ar |
| 20 | 92 | TRCN0000022609 | Araf |
| | 93 | TRCN0000022610 | Araf |
| | 94 | TRCN0000022611 | Araf |
| | 95 | TRCN0000022612 | Araf |
| | 96 | TRCN0000022613 | Araf |
| 21 | 97 | TRCN0000109960 | Arhgef12 |
| | 98 | TRCN0000109961 | Arhgef12 |
| | 99 | TRCN0000109962 | Arhgef12 |
| | 100 | TRCN0000109963 | Arhgef12 |
| | 101 | TRCN0000109964 | Arhgef12 |
| 22 | 102 | TRCN0000075553 | Atf5 |
| | 103 | TRCN0000075554 | Atf5 |
| | 104 | TRCN0000075555 | Atf5 |
| | 105 | TRCN0000075556 | Atf5 |
| | 106 | TRCN0000075557 | Atf5 |
| 23 | 107 | TRCN0000012643 | Atm |
| | 108 | TRCN0000012644 | Atm |
| | 109 | TRCN0000012645 | Atm |
| | 110 | TRCN0000012646 | Atm |
| | 111 | TRCN0000012647 | Atm |
| 24 | 112 | TRCN0000101520 | Atp10d |
| | 113 | TRCN0000101521 | Atp10d |
| | 114 | TRCN0000101522 | Atp10d |
| | 115 | TRCN0000101523 | Atp10d |
| 25 | 116 | TRCN0000115396 | Azin1 |
| | 117 | TRCN0000115397 | Azin1 |
| | 118 | TRCN0000115398 | Azin1 |
| | 119 | TRCN0000115399 | Azin1 |
| | 120 | TRCN0000115400 | Azin1 |
| 26 | 121 | TRCN0000070508 | Barx2 |
| | 122 | TRCN0000070509 | Barx2 |
| | 123 | TRCN0000070510 | Barx2 |
| | 124 | TRCN0000070511 | Barx2 |
| | 125 | TRCN0000070512 | Barx2 |
| 27 | 126 | TRCN0000004678 | Bcl2 |
| | 127 | TRCN0000004679 | Bcl2 |
| | 128 | TRCN0000004680 | Bcl2 |
| | 129 | TRCN0000004681 | Bcl2 |
| 28 | 130 | TRCN0000042553 | Bcl3 |
| | 131 | TRCN0000042554 | Bcl3 |
| | 132 | TRCN0000042555 | Bcl3 |
| | 133 | TRCN0000042556 | Bcl3 |
| | 134 | TRCN0000042557 | Bcl3 |
| 29 | 135 | TRCN0000012563 | Bmi1 |
| | 136 | TRCN0000012564 | Bmi1 |
| | 137 | TRCN0000012565 | Bmi1 |
| | 138 | TRCN0000012566 | Bmi1 |
| | 139 | TRCN0000012567 | Bmi1 |
| 30 | 140 | TRCN0000025877 | Bmp2 |
| | 141 | TRCN0000025878 | Bmp2 |
| | 142 | TRCN0000025923 | Bmp2 |
| | 143 | TRCN0000025939 | Bmp2 |
| | 144 | TRCN0000025949 | Bmp2 |
| 31 | 145 | TRCN0000025875 | Bmp4 |
| | 146 | TRCN0000025905 | Bmp4 |
| | 147 | TRCN0000025922 | Bmp4 |
| | 148 | TRCN0000025936 | Bmp4 |
| | 149 | TRCN0000025957 | Bmp4 |
| 32 | 150 | TRCN0000022619 | Bmpr1a |
| | 151 | TRCN0000022620 | Bmpr1a |
| | 152 | TRCN0000022621 | Bmpr1a |
| | 153 | TRCN0000022622 | Bmpr1a |
| | 154 | TRCN0000022623 | Bmpr1a |
| 33 | 155 | TRCN0000022529 | Bmpr2 |
| | 156 | TRCN0000022530 | Bmpr2 |
| | 157 | TRCN0000022531 | Bmpr2 |
| | 158 | TRCN0000022532 | Bmpr2 |
| | 159 | TRCN0000022533 | Bmpr2 |
| 34 | 160 | TRCN0000009687 | Bnip3 |
| | 161 | TRCN0000009688 | Bnip3 |
| | 162 | TRCN0000009689 | Bnip3 |
| | 163 | TRCN0000009690 | Bnip3 |
| | 164 | TRCN0000009691 | Bnip3 |
| 35 | 165 | TRCN0000022589 | Braf |
| | 166 | TRCN0000022590 | Braf |
| | 167 | TRCN0000022591 | Braf |
| | 168 | TRCN0000022592 | Braf |
| | 169 | TRCN0000022593 | Braf |
| 36 | 170 | TRCN0000042558 | Brca1 |
| | 171 | TRCN0000042559 | Brca1 |
| | 172 | TRCN0000042560 | Brca1 |
| | 173 | TRCN0000042561 | Brca1 |
| | 174 | TRCN0000042562 | Brca1 |
| 37 | 175 | TRCN0000071008 | Brca2 |
| | 176 | TRCN0000071009 | Brca2 |
| | 177 | TRCN0000071010 | Brca2 |
| | 178 | TRCN0000071011 | Brca2 |
| | 179 | TRCN0000071012 | Brca2 |
| 38 | 180 | TRCN0000103285 | C130053K05Rik |
| | 181 | TRCN0000103286 | C130053K05Rik |
| | 182 | TRCN0000103287 | C130053K05Rik |
| | 183 | TRCN0000103288 | C130053K05Rik |
| | 184 | TRCN0000103289 | C130053K05Rik |
| 39 | 185 | TRCN0000024114 | Camk1d |
| | 186 | TRCN0000024115 | Camk1d |
| | 187 | TRCN0000024116 | Camk1d |
| | 188 | TRCN0000024117 | Camk1d |
| | 189 | TRCN0000024118 | Camk1d |
| 40 | 190 | TRCN0000114461 | Car2 |
| | 191 | TRCN0000114462 | Car2 |
| | 192 | TRCN0000114463 | Car2 |
| | 193 | TRCN0000114464 | Car2 |
| | 194 | TRCN0000114465 | Car2 |
| 41 | 195 | TRCN0000012243 | Casp8 |
| | 196 | TRCN0000012244 | Casp8 |
| | 197 | TRCN0000012245 | Casp8 |
| | 198 | TRCN0000012246 | Casp8 |
| | 199 | TRCN0000012247 | Casp8 |
| 42 | 200 | TRCN0000042568 | Cbl |
| | 201 | TRCN0000042569 | Cbl |
| | 202 | TRCN0000042570 | Cbl |
| | 203 | TRCN0000042571 | Cbl |
| | 204 | TRCN0000042572 | Cbl |
| 43 | 205 | TRCN0000071028 | Cbx1 |
| | 206 | TRCN0000071029 | Cbx1 |
| | 207 | TRCN0000071030 | Cbx1 |
| | 208 | TRCN0000071031 | Cbx1 |
| | 209 | TRCN0000071032 | Cbx1 |
| 44 | 210 | TRCN0000071048 | Cbx5 |
| | 211 | TRCN0000071049 | Cbx5 |
| | 212 | TRCN0000071050 | Cbx5 |
| | 213 | TRCN0000071051 | Cbx5 |
| | 214 | TRCN0000071052 | Cbx5 |
| 45 | 215 | TRCN0000176503 | Ccdc39 |
| | 216 | TRCN0000176967 | Ccdc39 |
| | 217 | TRCN0000177337 | Ccdc39 |
| | 218 | TRCN0000182114 | Ccdc39 |
| | 219 | TRCN0000182268 | Ccdc39 |
| 46 | 220 | TRCN0000011978 | Ccnd3 |
| | 221 | TRCN0000011979 | Ccnd3 |
| | 222 | TRCN0000011980 | Ccnd3 |
| | 223 | TRCN0000011981 | Ccnd3 |
| 47 | 224 | TRCN0000119627 | Cd320 |
| | 225 | TRCN0000119629 | Cd320 |
| | 226 | TRCN0000119630 | Cd320 |
| | 227 | TRCN0000119631 | Cd320 |
| 48 | 228 | TRCN0000065353 | Cd44 |
| | 229 | TRCN0000065354 | Cd44 |
| | 230 | TRCN0000065355 | Cd44 |
| | 231 | TRCN0000065356 | Cd44 |
| | 232 | TRCN0000065357 | Cd44 |
| 49 | 233 | TRCN0000030109 | Cdc14b |
| | 234 | TRCN0000030110 | Cdc14b |
| | 235 | TRCN0000030111 | Cdc14b |
| | 236 | TRCN0000030112 | Cdc14b |
| | 237 | TRCN0000030113 | Cdc14b |
| 50 | 238 | TRCN0000042578 | Cdh1 |
| | 239 | TRCN0000042579 | Cdh1 |
| | 240 | TRCN0000042580 | Cdh1 |
| | 241 | TRCN0000042581 | Cdh1 |
| | 242 | TRCN0000042582 | Cdh1 |
| 51 | 243 | TRCN0000094534 | Cdh12 |
| | 244 | TRCN0000094535 | Cdh12 |
| | 245 | TRCN0000094536 | Cdh12 |
| | 246 | TRCN0000094537 | Cdh12 |
| | 247 | TRCN0000094538 | Cdh12 |
| 52 | 248 | TRCN0000094729 | Cdh4 |
| | 249 | TRCN0000094730 | Cdh4 |
| | 250 | TRCN0000094731 | Cdh4 |
| | 251 | TRCN0000094732 | Cdh4 |
| | 252 | TRCN0000094733 | Cdh4 |
| 53 | 253 | TRCN0000094894 | Cdh5 |
| | 254 | TRCN0000094895 | Cdh5 |
| | 255 | TRCN0000094896 | Cdh5 |
| | 256 | TRCN0000094897 | Cdh5 |
| | 257 | TRCN0000094898 | Cdh5 |
| 54 | 258 | TRCN0000094784 | Cdh7 |
| | 259 | TRCN0000094785 | Cdh7 |
| | 260 | TRCN0000094786 | Cdh7 |
| | 261 | TRCN0000094787 | Cdh7 |
| | 262 | TRCN0000094788 | Cdh7 |
| 55 | 263 | TRCN0000023174 | Cdk4 |
| | 264 | TRCN0000023175 | Cdk4 |
| | 265 | TRCN0000023176 | Cdk4 |
| | 266 | TRCN0000023177 | Cdk4 |
| | 267 | TRCN0000023178 | Cdk4 |
| 56 | 268 | TRCN0000042583 | Cdkn1a |
| | 269 | TRCN0000042585 | Cdkn1a |
| | 270 | TRCN0000042586 | Cdkn1a |
| | 271 | TRCN0000042587 | Cdkn1a |
| | 272 | TRCN0000054898 | Cdkn1a |
| | 273 | TRCN0000054899 | Cdkn1a |
| | 274 | TRCN0000054900 | Cdkn1a |
| | 275 | TRCN0000054901 | Cdkn1a |
| | 276 | TRCN0000054902 | Cdkn1a |
| 57 | 277 | TRCN0000071063 | Cdkn1b |
| | 278 | TRCN0000071064 | Cdkn1b |
| | 279 | TRCN0000071066 | Cdkn1b |
| | 280 | TRCN0000071067 | Cdkn1b |
| 58 | 281 | TRCN0000042588 | Cdkn1c |
| | 282 | TRCN0000042589 | Cdkn1c |
| | 283 | TRCN0000042590 | Cdkn1c |
| | 284 | TRCN0000042592 | Cdkn1c |
| 59 | 285 | TRCN0000077813 | Cdkn2a |
| | 286 | TRCN0000077815 | Cdkn2a |
| | 287 | TRCN0000077816 | Cdkn2a |
| 60 | 288 | TRCN0000042598 | Cdkn2b |
| | 289 | TRCN0000042599 | Cdkn2b |
| | 290 | TRCN0000042600 | Cdkn2b |
| | 291 | TRCN0000042601 | Cdkn2b |
| | 292 | TRCN0000042602 | Cdkn2b |
| 61 | 293 | TRCN0000085088 | Cdkn2d |
| | 294 | TRCN0000085089 | Cdkn2d |
| | 295 | TRCN0000085090 | Cdkn2d |
| | 296 | TRCN0000085091 | Cdkn2d |
| | 297 | TRCN0000085092 | Cdkn2d |
| 62 | 298 | TRCN0000071654 | Cebpd |
| | 299 | TRCN0000071655 | Cebpd |
| | 300 | TRCN0000071657 | Cebpd |
| 63 | 301 | TRCN0000094949 | Celsr3 |
| | 302 | TRCN0000094950 | Celsr3 |
| | 303 | TRCN0000094951 | Celsr3 |
| | 304 | TRCN0000094952 | Celsr3 |
| | 305 | TRCN0000094953 | Celsr3 |
| 64 | 306 | TRCN0000179809 | Cep55 |
| | 307 | TRCN0000182908 | Cep55 |
| | 308 | TRCN0000183083 | Cep55 |
| | 309 | TRCN0000183560 | Cep55 |
| 65 | 310 | TRCN0000012648 | Chek1 |
| | 311 | TRCN0000012649 | Chek1 |
| | 312 | TRCN0000012650 | Chek1 |
| | 313 | TRCN0000012651 | Chek1 |
| | 314 | TRCN0000012652 | Chek1 |
| 66 | 315 | TRCN0000012653 | Chek2 |
| | 316 | TRCN0000012654 | Chek2 |
| | 317 | TRCN0000012655 | Chek2 |
| | 318 | TRCN0000012656 | Chek2 |
| | 319 | TRCN0000012657 | Chek2 |
| 67 | 320 | TRCN0000103290 | Chpt1 |
| | 321 | TRCN0000103292 | Chpt1 |
| | 322 | TRCN0000103293 | Chpt1 |
| | 323 | TRCN0000103294 | Chpt1 |
| 68 | 324 | TRCN0000025883 | Chrd |
| | 325 | TRCN0000025906 | Chrd |
| | 326 | TRCN0000025914 | Chrd |
| | 327 | TRCN0000025932 | Chrd |
| | 328 | TRCN0000025944 | Chrd |
| 69 | 329 | TRCN0000012348 | Chuk |
| | 330 | TRCN0000012349 | Chuk |
| | 331 | TRCN0000012350 | Chuk |
| | 332 | TRCN0000012351 | Chuk |
| | 333 | TRCN0000012352 | Chuk |
| 70 | 334 | TRCN0000069708 | Clca2 |
| | 335 | TRCN0000069709 | Clca2 |
| | 336 | TRCN0000069710 | Clca2 |
| | 337 | TRCN0000069711 | Clca2 |
| | 338 | TRCN0000069712 | Clca2 |
| 71 | 339 | TRCN0000069738 | Clic1 |
| | 340 | TRCN0000069739 | Clic1 |
| | 341 | TRCN0000069740 | Clic1 |
| | 342 | TRCN0000069741 | Clic1 |
| 72 | 343 | TRCN0000023189 | Clk3 |
| | 344 | TRCN0000023190 | Clk3 |
| | 345 | TRCN0000023191 | Clk3 |
| | 346 | TRCN0000023192 | Clk3 |
| | 347 | TRCN0000023193 | Clk3 |
| 73 | 348 | TRCN0000023194 | Clk4 |
| | 349 | TRCN0000023195 | Clk4 |
| | 350 | TRCN0000023196 | Clk4 |
| | 351 | TRCN0000023197 | Clk4 |
| | 352 | TRCN0000023198 | Clk4 |
| 74 | 353 | TRCN0000094734 | Clstn2 |
| | 354 | TRCN0000094735 | Clstn2 |
| | 355 | TRCN0000094736 | Clstn2 |
| | 356 | TRCN0000094737 | Clstn2 |
| | 357 | TRCN0000094738 | Clstn2 |
| 75 | 358 | TRCN0000039014 | Cntn1 |
| | 359 | TRCN0000039015 | Cntn1 |
| | 360 | TRCN0000039016 | Cntn1 |
| | 361 | TRCN0000039017 | Cntn1 |
| | 362 | TRCN0000039018 | Cntn1 |
| 76 | 363 | TRCN0000113645 | Cntn3 |
| | 364 | TRCN0000113646 | Cntn3 |
| | 365 | TRCN0000113647 | Cntn3 |
| | 366 | TRCN0000113648 | Cntn3 |
| | 367 | TRCN0000113649 | Cntn3 |
| 77 | 368 | TRCN0000094359 | Cntnap1 |
| | 369 | TRCN0000094360 | Cntnap1 |
| | 370 | TRCN0000094361 | Cntnap1 |
| | 371 | TRCN0000094362 | Cntnap1 |
| | 372 | TRCN0000094363 | Cntnap1 |
| 78 | 373 | TRCN0000094969 | Cntnap2 |
| | 374 | TRCN0000094970 | Cntnap2 |
| | 375 | TRCN0000094971 | Cntnap2 |
| | 376 | TRCN0000094972 | Cntnap2 |
| | 377 | TRCN0000094973 | Cntnap2 |
| 79 | 378 | TRCN0000094539 | Cntnap4 |
| | 379 | TRCN0000094540 | Cntnap4 |
| 80 | 380 | TRCN0000090503 | Col1a1 |
| | 381 | TRCN0000090504 | Col1a1 |
| | 382 | TRCN0000090505 | Col1a1 |
| | 383 | TRCN0000090506 | Col1a1 |
| | 384 | TRCN0000090507 | Col1a1 |
| 81 | 385 | TRCN0000090043 | Col1a2 |
| | 386 | TRCN0000090044 | Col1a2 |
| | 387 | TRCN0000090045 | Col1a2 |
| | 388 | TRCN0000090046 | Col1a2 |
| | 389 | TRCN0000090047 | Col1a2 |
| 82 | 390 | TRCN0000091163 | Col22a1 |
| | 391 | TRCN0000091164 | Col22a1 |
| | 392 | TRCN0000091165 | Col22a1 |
| | 393 | TRCN0000091166 | Col22a1 |
| | 394 | TRCN0000091167 | Col22a1 |
| 83 | 395 | TRCN0000091483 | Col3a1 |
| | 396 | TRCN0000091484 | Col3a1 |
| | 397 | TRCN0000091485 | Col3a1 |
| | 398 | TRCN0000091486 | Col3a1 |
| | 399 | TRCN0000091487 | Col3a1 |
| 84 | 400 | TRCN0000031319 | Cpxm2 |
| | 401 | TRCN0000031320 | Cpxm2 |
| | 402 | TRCN0000031321 | Cpxm2 |
| | 403 | TRCN0000031322 | Cpxm2 |
| | 404 | TRCN0000031323 | Cpxm2 |
| 85 | 405 | TRCN0000105235 | Crabp2 |
| | 406 | TRCN0000105236 | Crabp2 |
| | 407 | TRCN0000105237 | Crabp2 |
| | 408 | TRCN0000105238 | Crabp2 |
| | 409 | TRCN0000105239 | Crabp2 |
| 86 | 410 | TRCN0000042603 | Crk |
| | 411 | TRCN0000042604 | Crk |
| | 412 | TRCN0000042606 | Crk |
| | 413 | TRCN0000042607 | Crk |
| 87 | 414 | TRCN0000023734 | Csk |
| | 415 | TRCN0000023735 | Csk |
| | 416 | TRCN0000023736 | Csk |
| | 417 | TRCN0000023737 | Csk |
| | 418 | TRCN0000023738 | Csk |
| 88 | 419 | TRCN0000087303 | Csmd3 |
| | 420 | TRCN0000087304 | Csmd3 |
| | 421 | TRCN0000087305 | Csmd3 |
| | 422 | TRCN0000087306 | Csmd3 |
| | 423 | TRCN0000087307 | Csmd3 |
| 89 | 424 | TRCN0000080278 | Cst6 |
| | 425 | TRCN0000080279 | Cst6 |
| | 426 | TRCN0000080280 | Cst6 |
| | 427 | TRCN0000080281 | Cst6 |
| | 428 | TRCN0000080282 | Cst6 |
| 90 | 429 | TRCN0000039019 | Ctcf |
| | 430 | TRCN0000039020 | Ctcf |
| | 431 | TRCN0000039021 | Ctcf |
| | 432 | TRCN0000039022 | Ctcf |
| | 433 | TRCN0000039023 | Ctcf |
| 91 | 434 | TRCN0000109665 | Ctgf |
| | 435 | TRCN0000109666 | Ctgf |
| | 436 | TRCN0000109667 | Ctgf |
| | 437 | TRCN0000109668 | Ctgf |
| | 438 | TRCN0000109669 | Ctgf |
| 92 | 439 | TRCN0000065368 | Cxcl14 |
| | 440 | TRCN0000065369 | Cxcl14 |
| | 441 | TRCN0000065370 | Cxcl14 |
| | 442 | TRCN0000065371 | Cxcl14 |
| | 443 | TRCN0000065372 | Cxcl14 |
| 93 | 444 | TRCN0000067258 | Cxcl2 |
| | 445 | TRCN0000067259 | Cxcl2 |
| | 446 | TRCN0000067260 | Cxcl2 |
| | 447 | TRCN0000067261 | Cxcl2 |
| 94 | 448 | TRCN0000028678 | Cxcr4 |
| | 449 | TRCN0000028704 | Cxcr4 |
| | 450 | TRCN0000028724 | Cxcr4 |
| | 451 | TRCN0000028749 | Cxcr4 |
| | 452 | TRCN0000028750 | Cxcr4 |
| 95 | 453 | TRCN0000125700 | Cyp4f16 |
| | 454 | TRCN0000125701 | Cyp4f16 |
| | 455 | TRCN0000125702 | Cyp4f16 |
| | 456 | TRCN0000125703 | Cyp4f16 |
| 96 | 457 | TRCN0000103750 | Ddx3x |
| | 458 | TRCN0000103751 | Ddx3x |
| | 459 | TRCN0000103752 | Ddx3x |
| | 460 | TRCN0000103753 | Ddx3x |
| | 461 | TRCN0000103754 | Ddx3x |
| 97 | 462 | TRCN0000099475 | Defb6 |
| | 463 | TRCN0000099476 | Defb6 |
| | 464 | TRCN0000099477 | Defb6 |
| | 465 | TRCN0000099478 | Defb6 |
| 98 | 466 | TRCN0000028845 | Dll1 |
| | 467 | TRCN0000028864 | Dll1 |
| | 468 | TRCN0000028865 | Dll1 |
| | 469 | TRCN0000028890 | Dll1 |
| | 470 | TRCN0000028910 | Dll1 |
| 99 | 471 | TRCN0000028875 | Dll3 |
| | 472 | TRCN0000028879 | Dll3 |
| | 473 | TRCN0000028896 | Dll3 |
| | 474 | TRCN0000028907 | Dll3 |
| | 475 | TRCN0000028924 | Dll3 |
| 100 | 476 | TRCN0000028894 | Dll4 |
| | 477 | TRCN0000028916 | Dll4 |
| | 478 | TRCN0000028928 | Dll4 |
| 101 | 479 | TRCN0000070598 | Dlx2 |
| | 480 | TRCN0000070599 | Dlx2 |
| | 481 | TRCN0000070600 | Dlx2 |
| | 482 | TRCN0000070601 | Dlx2 |
| | 483 | TRCN0000070602 | Dlx2 |
| 102 | 484 | TRCN0000070608 | Dlx3 |
| | 485 | TRCN0000070609 | Dlx3 |
| | 486 | TRCN0000070610 | Dlx3 |
| | 487 | TRCN0000070611 | Dlx3 |
| | 488 | TRCN0000070612 | Dlx3 |
| 103 | 489 | TRCN0000070628 | Dlx5 |
| | 490 | TRCN0000070629 | Dlx5 |
| | 491 | TRCN0000070630 | Dlx5 |
| | 492 | TRCN0000070632 | Dlx5 |
| 104 | 493 | TRCN0000086488 | Dmrta2 |
| | 494 | TRCN0000086489 | Dmrta2 |
| | 495 | TRCN0000086490 | Dmrta2 |
| | 496 | TRCN0000086491 | Dmrta2 |
| 105 | 497 | TRCN0000008562 | Dnajb9 |
| | 498 | TRCN0000008563 | Dnajb9 |
| | 499 | TRCN0000008564 | Dnajb9 |
| | 500 | TRCN0000008565 | Dnajb9 |
| | 501 | TRCN0000008566 | Dnajb9 |
| 106 | 502 | TRCN0000039024 | Dnmt1 |
| | 503 | TRCN0000039025 | Dnmt1 |
| | 504 | TRCN0000039026 | Dnmt1 |
| | 505 | TRCN0000039027 | Dnmt1 |
| | 506 | TRCN0000039028 | Dnmt1 |
| 107 | 507 | TRCN0000039029 | Dnmt2 |
| | 508 | TRCN0000039030 | Dnmt2 |
| | 509 | TRCN0000039031 | Dnmt2 |
| | 510 | TRCN0000039032 | Dnmt2 |
| | 511 | TRCN0000039033 | Dnmt2 |
| 108 | 512 | TRCN0000039034 | Dnmt3a |
| | 513 | TRCN0000039035 | Dnmt3a |
| | 514 | TRCN0000039036 | Dnmt3a |
| | 515 | TRCN0000039037 | Dnmt3a |
| | 516 | TRCN0000039038 | Dnmt3a |
| 109 | 517 | TRCN0000039104 | Dnmt31 |
| | 518 | TRCN0000039105 | Dnmt31 |
| | 519 | TRCN0000039106 | Dnmt31 |
| | 520 | TRCN0000039107 | Dnmt31 |
| | 521 | TRCN0000039108 | Dnmt31 |
| 110 | 522 | TRCN0000054348 | Dusp4 |
| | 523 | TRCN0000054349 | Dusp4 |
| | 524 | TRCN0000054350 | Dusp4 |
| | 525 | TRCN0000054351 | Dusp4 |
| | 526 | TRCN0000054352 | Dusp4 |
| 111 | 527 | TRCN0000023479 | Egfr |
| | 528 | TRCN0000023480 | Egfr |
| | 529 | TRCN0000023481 | Egfr |
| | 530 | TRCN0000023482 | Egfr |
| | 531 | TRCN0000023483 | Egfr |
| | 532 | TRCN0000055218 | Egfr |
| | 533 | TRCN0000055219 | Egfr |
| | 534 | TRCN0000055220 | Egfr |
| | 535 | TRCN0000055221 | Egfr |
| | 536 | TRCN0000055222 | Egfr |
| 112 | 537 | TRCN0000009749 | Egln3 |
| | 538 | TRCN0000009750 | Egln3 |
| | 539 | TRCN0000009751 | Egln3 |
| | 540 | TRCN0000009752 | Egln3 |
| | 541 | TRCN0000009753 | Egln3 |
| 113 | 542 | TRCN0000081623 | Egr1 |
| | 543 | TRCN0000081624 | Egr1 |
| | 544 | TRCN0000081625 | Egr1 |
| | 545 | TRCN0000081626 | Egr1 |
| | 546 | TRCN0000081627 | Egr1 |
| 114 | 547 | TRCN0000081678 | Egr2 |
| | 548 | TRCN0000081679 | Egr2 |
| | 549 | TRCN0000081680 | Egr2 |
| | 550 | TRCN0000081681 | Egr2 |
| | 551 | TRCN0000081682 | Egr2 |
| 115 | 552 | TRCN0000081788 | Ehf |
| | 553 | TRCN0000081789 | Ehf |
| | 554 | TRCN0000081790 | Ehf |
| | 555 | TRCN0000081791 | Ehf |
| | 556 | TRCN0000081792 | Ehf |
| 116 | 557 | TRCN0000081938 | Elf5 |
| | 558 | TRCN0000081939 | Elf5 |
| | 559 | TRCN0000081940 | Elf5 |
| | 560 | TRCN0000081941 | Elf5 |
| | 561 | TRCN0000081942 | Elf5 |
| 117 | 562 | TRCN0000042643 | Elk3 |
| | 563 | TRCN0000042644 | Elk3 |
| | 564 | TRCN0000042645 | Elk3 |
| | 565 | TRCN0000042646 | Elk3 |
| | 566 | TRCN0000042647 | Elk3 |
| 118 | 567 | TRCN0000023679 | Epha7 |
| | 568 | TRCN0000023680 | Epha7 |
| | 569 | TRCN0000023681 | Epha7 |
| | 570 | TRCN0000023682 | Epha7 |
| | 571 | TRCN0000023683 | Epha7 |
| 119 | 572 | TRCN0000092273 | Eps8 |
| | 573 | TRCN0000092274 | Eps8 |
| | 574 | TRCN0000092275 | Eps8 |
| | 575 | TRCN0000092276 | Eps8 |
| | 576 | TRCN0000092277 | Eps8 |
| 120 | 577 | TRCN0000190945 | Esm1 |
| | 578 | TRCN0000192471 | Esm1 |
| | 579 | TRCN0000192502 | Esm1 |
| | 580 | TRCN0000192617 | Esm1 |
| 121 | 581 | TRCN0000026176 | Esr1 |
| | 582 | TRCN0000026184 | Esr1 |
| | 583 | TRCN0000026197 | Esr1 |
| | 584 | TRCN0000026201 | Esr1 |
| | 585 | TRCN0000026214 | Esr1 |
| 122 | 586 | TRCN0000026150 | Esr2 |
| | 587 | TRCN0000026170 | Esr2 |
| | 588 | TRCN0000026192 | Esr2 |
| | 589 | TRCN0000026215 | Esr2 |
| 123 | 590 | TRCN0000111725 | Exoc4 |
| | 591 | TRCN0000111726 | Exoc4 |
| | 592 | TRCN0000111727 | Exoc4 |
| | 593 | TRCN0000111728 | Exoc4 |
| | 594 | TRCN0000111729 | Exoc4 |
| 124 | 595 | TRCN0000095694 | Ezh1 |
| | 596 | TRCN0000095695 | Ezh1 |
| | 597 | TRCN0000095696 | Ezh1 |
| | 598 | TRCN0000095697 | Ezh1 |
| | 599 | TRCN0000095698 | Ezh1 |
| 125 | 600 | TRCN0000039039 | Ezh2 |
| | 601 | TRCN0000039040 | Ezh2 |
| | 602 | TRCN0000039041 | Ezh2 |
| | 603 | TRCN0000039042 | Ezh2 |
| | 604 | TRCN0000039043 | Ezh2 |
| 126 | 605 | TRCN0000105190 | Fabp3 |
| | 606 | TRCN0000105191 | Fabp3 |
| | 607 | TRCN0000105192 | Fabp3 |
| | 608 | TRCN0000105193 | Fabp3 |
| | 609 | TRCN0000105194 | Fabp3 |
| 127 | 610 | TRCN0000105185 | Fabp4 |
| | 611 | TRCN0000105186 | Fabp4 |
| | 612 | TRCN0000105187 | Fabp4 |
| | 613 | TRCN0000105188 | Fabp4 |
| | 614 | TRCN0000105189 | Fabp4 |
| 128 | 615 | NM_010634.1-149s1c1 | Fabp5 |
| | 616 | NM_010634.1-592s1c1 | Fabp5 |
| | 617 | TRCN0000011894 | Fabp5 |
| | 618 | TRCN0000011896 | Fabp5 |
| | 619 | TRCN0000011897 | Fabp5 |
| 129 | 620 | TRCN0000114336 | Fads2 |
| | 621 | TRCN0000114337 | Fads2 |
| | 622 | TRCN0000114338 | Fads2 |
| | 623 | TRCN0000114340 | Fads2 |
| 130 | 624 | TRCN0000173476 | Fancm |
| | 625 | TRCN0000173798 | Fancm |
| | 626 | TRCN0000175001 | Fancm |
| | 627 | TRCN0000176065 | Fancm |
| | 628 | TRCN0000176066 | Fancm |
| 131 | 629 | TRCN0000094844 | Fath |
| | 630 | TRCN0000094845 | Fath |
| | 631 | TRCN0000094846 | Fath |
| | 632 | TRCN0000094847 | Fath |
| | 633 | TRCN0000094848 | Fath |
| 132 | 634 | TRCN0000012828 | Fbxw7 |
| | 635 | TRCN0000012829 | Fbxw7 |
| | 636 | TRCN0000012830 | Fbxw7 |
| | 637 | TRCN0000012831 | Fbxw7 |
| | 638 | TRCN0000012832 | Fbxw7 |
| 133 | 639 | TRCN0000004653 | Ffar1 |
| | 640 | TRCN0000004654 | Ffar1 |
| | 641 | TRCN0000004655 | Ffar1 |
| 134 | 642 | TRCN0000009606 | Flt1 |
| | 643 | TRCN0000009607 | Flt1 |
| | 644 | TRCN0000009608 | Flt1 |
| | 645 | TRCN0000009609 | Flt1 |
| | 646 | TRCN0000009610 | Flt1 |
| 135 | 647 | TRCN0000023739 | Flt3 |
| | 648 | TRCN0000023740 | Flt3 |
| | 649 | TRCN0000023741 | Flt3 |
| | 650 | TRCN0000023742 | Flt3 |
| | 651 | TRCN0000023743 | Flt3 |
| 136 | 652 | TRCN0000023754 | Flt4 |
| | 653 | TRCN0000023755 | Flt4 |
| | 654 | TRCN0000023756 | Flt4 |
| | 655 | TRCN0000023757 | Flt4 |
| | 656 | TRCN0000023758 | Flt4 |
| 137 | 657 | TRCN0000120512 | Fmn2 |
| | 658 | TRCN0000120513 | Fmn2 |
| | 659 | TRCN0000120514 | Fmn2 |
| | 660 | TRCN0000120515 | Fmn2 |
| | 661 | TRCN0000120516 | Fmn2 |
| 138 | 662 | TRCN0000084288 | Foxj2 |
| | 663 | TRCN0000084289 | Foxj2 |
| | 664 | TRCN0000084290 | Foxj2 |
| | 665 | TRCN0000084291 | Foxj2 |
| | 666 | TRCN0000084292 | Foxj2 |
| 139 | 667 | TRCN0000072003 | Foxp1 |
| | 668 | TRCN0000072004 | Foxp1 |
| | 669 | TRCN0000072005 | Foxp1 |
| | 670 | TRCN0000072006 | Foxp1 |
| | 671 | TRCN0000072007 | Foxp1 |
| 140 | 672 | TRCN0000108925 | Fscn1 |
| | 673 | TRCN0000108926 | Fscn1 |
| | 674 | TRCN0000108927 | Fscn1 |
| | 675 | TRCN0000108928 | Fscn1 |
| | 676 | TRCN0000108929 | Fscn1 |
| 141 | 677 | TRCN0000085478 | Gata3 |
| | 678 | TRCN0000085479 | Gata3 |
| | 679 | TRCN0000085480 | Gata3 |
| | 680 | TRCN0000085481 | Gata3 |
| | 681 | TRCN0000085482 | Gata3 |
| 142 | 682 | TRCN0000068823 | Gjb5 |
| | 683 | TRCN0000068824 | Gjb5 |
| | 684 | TRCN0000068825 | Gjb5 |
| | 685 | TRCN0000068826 | Gjb5 |
| | 686 | TRCN0000068827 | Gjb5 |
| 143 | 687 | TRCN0000027955 | Gpr56 |
| | 688 | TRCN0000027962 | Gpr56 |
| | 689 | TRCN0000027970 | Gpr56 |
| | 690 | TRCN0000027988 | Gpr56 |
| | 691 | TRCN0000027999 | Gpr56 |
| 144 | 692 | TRCN0000076528 | Gpx2 |
| | 693 | TRCN0000076529 | Gpx2 |
| | 694 | TRCN0000076530 | Gpx2 |
| | 695 | TRCN0000076531 | Gpx2 |
| | 696 | TRCN0000076532 | Gpx2 |
| 145 | 697 | TRCN0000103545 | Grhl3 |
| | 698 | TRCN0000103546 | Grhl3 |
| | 699 | TRCN0000103547 | Grhl3 |
| | 700 | TRCN0000103548 | Grhl3 |
| | 701 | TRCN0000103549 | Grhl3 |
| 146 | 702 | TRCN0000103040 | Grid1 |
| | 703 | TRCN0000103041 | Grid1 |
| | 704 | TRCN0000103042 | Grid1 |
| | 705 | TRCN0000103043 | Grid1 |
| | 706 | TRCN0000103044 | Grid1 |
| 147 | 707 | TRCN0000012613 | Gsk3b |
| | 708 | TRCN0000012614 | Gsk3b |
| | 709 | TRCN0000012615 | Gsk3b |
| | 710 | TRCN0000012616 | Gsk3b |
| | 711 | TRCN0000012617 | Gsk3b |
| 148 | 712 | TRCN0000103310 | Gsta1 |
| | 713 | TRCN0000103311 | Gsta1 |
| | 714 | TRCN0000103312 | Gsta1 |
| | 715 | TRCN0000103313 | Gsta1 |
| | 716 | TRCN0000103314 | Gsta1 |
| 149 | 717 | TRCN0000103295 | Gsta2 |
| | 718 | TRCN0000103296 | Gsta2 |
| | 719 | TRCN0000103297 | Gsta2 |
| | 720 | TRCN0000103298 | Gsta2 |
| | 721 | TRCN0000103299 | Gsta2 |
| 150 | 722 | TRCN0000103280 | Gsta3 |
| | 723 | TRCN0000103281 | Gsta3 |
| | 724 | TRCN0000103282 | Gsta3 |
| | 725 | TRCN0000103283 | Gsta3 |
| | 726 | TRCN0000103284 | Gsta3 |
| 151 | 727 | TRCN0000103430 | Gsta4 |
| | 728 | TRCN0000103431 | Gsta4 |
| | 729 | TRCN0000103432 | Gsta4 |
| | 730 | TRCN0000103433 | Gsta4 |
| | 731 | TRCN0000103434 | Gsta4 |
| 152 | 732 | TRCN0000103240 | Gstm1 |
| | 733 | TRCN0000103241 | Gstm1 |
| | 734 | TRCN0000103242 | Gstm1 |
| | 735 | TRCN0000103243 | Gstm1 |
| | 736 | TRCN0000103244 | Gstm1 |
| 153 | 737 | TRCN0000103160 | Gstm2 |
| | 738 | TRCN0000103161 | Gstm2 |
| | 739 | TRCN0000103162 | Gstm2 |
| | 740 | TRCN0000103163 | Gstm2 |
| | 741 | TRCN0000103164 | Gstm2 |
| 154 | 742 | TRCN0000028854 | Hes1 |
| | 743 | TRCN0000028855 | Hes1 |
| | 744 | TRCN0000028881 | Hes1 |
| | 745 | TRCN0000028925 | Hes1 |
| | 746 | TRCN0000028927 | Hes1 |
| 155 | 747 | TRCN0000096954 | Hist1h2bh |
| | 748 | TRCN0000096955 | Hist1h2bh |
| | 749 | TRCN0000096956 | Hist1h2bh |
| | 750 | TRCN0000096957 | Hist1h2bh |
| | 751 | TRCN0000096958 | Hist1h2bh |
| 156 | 752 | TRCN0000126044 | Hmga2 |
| | 753 | TRCN0000126045 | Hmga2 |
| | 754 | TRCN0000126046 | Hmga2 |
| | 755 | TRCN0000126047 | Hmga2 |
| | 756 | TRCN0000126048 | Hmga2 |
| 157 | 757 | TRCN0000075583 | Hmgb2 |
| | 758 | TRCN0000075584 | Hmgb2 |
| | 759 | TRCN0000075585 | Hmgb2 |
| | 760 | TRCN0000075586 | Hmgb2 |
| | 761 | TRCN0000075587 | Hmgb2 |
| 158 | 762 | TRCN0000070789 | Hoxa4 |
| | 763 | TRCN0000070790 | Hoxa4 |
| | 764 | TRCN0000070791 | Hoxa4 |
| | 765 | TRCN0000070792 | Hoxa4 |
| 159 | 766 | TRCN0000012518 | Hoxa5 |
| | 767 | TRCN0000012519 | Hoxa5 |
| | 768 | TRCN0000012520 | Hoxa5 |
| | 769 | TRCN0000012521 | Hoxa5 |
| | 770 | TRCN0000012522 | Hoxa5 |
| 160 | 771 | TRCN0000070863 | Hoxb6 |
| | 772 | TRCN0000070864 | Hoxb6 |
| | 773 | TRCN0000070865 | Hoxb6 |
| | 774 | TRCN0000070866 | Hoxb6 |
| | 775 | TRCN0000070867 | Hoxb6 |
| 161 | 776 | TRCN0000070888 | Hoxb9 |
| | 777 | TRCN0000070889 | Hoxb9 |
| | 778 | TRCN0000070890 | Hoxb9 |
| | 779 | TRCN0000070891 | Hoxb9 |
| | 780 | TRCN0000070892 | Hoxb9 |
| 162 | 781 | TRCN0000070908 | Hoxc13 |
| | 782 | TRCN0000070909 | Hoxc13 |
| | 783 | TRCN0000070910 | Hoxc13 |
| | 784 | TRCN0000070911 | Hoxc13 |
| 163 | 785 | TRCN0000070938 | Hoxc6 |
| | 786 | TRCN0000070939 | Hoxc6 |
| | 787 | TRCN0000070940 | Hoxc6 |
| | 788 | TRCN0000070941 | Hoxc6 |
| | 789 | TRCN0000070942 | Hoxc6 |
| 164 | 790 | TRCN0000070948 | Hoxc8 |
| | 791 | TRCN0000070949 | Hoxc8 |
| | 792 | TRCN0000070950 | Hoxc8 |
| | 793 | TRCN0000070951 | Hoxc8 |
| 165 | 794 | TRCN0000070468 | Hoxd9 |
| | 795 | TRCN0000070469 | Hoxd9 |
| | 796 | TRCN0000070470 | Hoxd9 |
| | 797 | TRCN0000070471 | Hoxd9 |
| | 798 | TRCN0000070472 | Hoxd9 |
| 166 | 799 | TRCN0000034379 | Hras1 |
| | 800 | TRCN0000034380 | Hras1 |
| | 801 | TRCN0000034381 | Hras1 |
| | 802 | TRCN0000034382 | Hras1 |
| | 803 | TRCN0000034383 | Hras1 |
| 167 | 804 | TRCN0000071433 | Id1 |
| | 805 | TRCN0000071435 | Id1 |
| | 806 | TRCN0000071437 | Id1 |
| 168 | 807 | TRCN0000071438 | Id3 |
| | 808 | TRCN0000071439 | Id3 |
| | 809 | TRCN0000071440 | Id3 |
| | 810 | TRCN0000071444 | Id4 |
| 169 | 811 | TRCN0000023489 | Igf1r |
| | 812 | TRCN0000023490 | Igf1r |
| | 813 | TRCN0000023491 | Igf1r |
| | 814 | TRCN0000023492 | Igf1r |
| | 815 | TRCN0000023493 | Igf1r |
| 170 | 816 | TRCN0000096759 | Igf2bp2 |
| | 817 | TRCN0000096760 | Igf2bp2 |
| | 818 | TRCN0000096761 | Igf2bp2 |
| | 819 | TRCN0000096762 | Igf2bp2 |
| | 820 | TRCN0000096763 | Igf2bp2 |
| 171 | 821 | TRCN0000012858 | Igfbp2 |
| | 822 | TRCN0000012859 | Igfbp2 |
| | 823 | TRCN0000012860 | Igfbp2 |
| | 824 | TRCN0000012861 | Igfbp2 |
| | 825 | TRCN0000012862 | Igfbp2 |
| 172 | 826 | TRCN0000026867 | Ikbkb |
| | 827 | TRCN0000026891 | Ikbkb |
| | 828 | TRCN0000026894 | Ikbkb |
| | 829 | TRCN0000026913 | Ikbkb |
| | 830 | TRCN0000026945 | Ikbkb |
| 173 | 831 | TRCN0000088808 | Ikbkg |
| | 832 | TRCN0000088809 | Ikbkg |
| | 833 | TRCN0000088810 | Ikbkg |
| | 834 | TRCN0000088811 | Ikbkg |
| | 835 | TRCN0000088812 | Ikbkg |
| 174 | 836 | TRCN0000068248 | Il1r2 |
| | 837 | TRCN0000068249 | Il1r2 |
| | 838 | TRCN0000068250 | Il1r2 |
| | 839 | TRCN0000068251 | Il1r2 |
| | 840 | TRCN0000068252 | Il1r2 |
| 175 | 841 | TRCN0000085328 | Irf6 |
| | 842 | TRCN0000085329 | Irf6 |
| | 843 | TRCN0000085330 | Irf6 |
| | 844 | TRCN0000085331 | Irf6 |
| | 845 | TRCN0000085332 | Irf6 |
| 176 | 846 | TRCN0000070478 | Irx1 |
| | 847 | TRCN0000070479 | Irx1 |
| | 848 | TRCN0000070480 | Irx1 |
| | 849 | TRCN0000070481 | Irx1 |
| | 850 | TRCN0000070482 | Irx1 |
| 177 | 851 | TRCN0000070403 | Irx4 |
| | 852 | TRCN0000070404 | Irx4 |
| | 853 | TRCN0000070405 | Irx4 |
| | 854 | TRCN0000070406 | Irx4 |
| | 855 | TRCN0000070407 | Irx4 |
| 178 | 856 | TRCN0000070418 | Irx5 |
| | 857 | TRCN0000070419 | Irx5 |
| | 858 | TRCN0000070420 | Irx5 |
| | 859 | TRCN0000070421 | Irx5 |
| | 860 | TRCN0000070422 | Irx5 |
| 179 | 861 | TRCN0000028850 | Jag1 |
| | 862 | TRCN0000028860 | Jag1 |
| | 863 | TRCN0000028869 | Jag1 |
| | 864 | TRCN0000028887 | Jag1 |
| | 865 | TRCN0000028933 | Jag1 |
| 180 | 866 | TRCN0000028871 | Jag2 |
| | 867 | TRCN0000028877 | Jag2 |
| | 868 | TRCN0000028897 | Jag2 |
| | 869 | TRCN0000028906 | Jag2 |
| 181 | 870 | TRCN0000075548 | Jub |
| | 871 | TRCN0000075549 | Jub |
| | 872 | TRCN0000075550 | Jub |
| | 873 | TRCN0000075551 | Jub |
| | 874 | TRCN0000075552 | Jub |
| 182 | 875 | TRCN0000055203 | Jun |
| | 876 | TRCN0000055204 | Jun |
| | 877 | TRCN0000055205 | Jun |
| | 878 | TRCN0000055206 | Jun |
| | 879 | TRCN0000055207 | Jun |
| 183 | 880 | TRCN0000069668 | Kctd8 |
| | 881 | TRCN0000069669 | Kctd8 |
| | 882 | TRCN0000069670 | Kctd8 |
| | 883 | TRCN0000069671 | Kctd8 |
| | 884 | TRCN0000069672 | Kctd8 |
| 184 | 885 | TRCN0000023744 | Kdr |
| | 886 | TRCN0000023745 | Kdr |
| | 887 | TRCN0000023746 | Kdr |
| | 888 | TRCN0000023747 | Kdr |
| | 889 | TRCN0000023748 | Kdr |
| 185 | 890 | TRCN0000071468 | Klf15 |
| | 891 | TRCN0000071469 | Klf15 |
| | 892 | TRCN0000071470 | Klf15 |
| | 893 | TRCN0000071471 | Klf15 |
| | 894 | TRCN0000071472 | Klf15 |
| 186 | 895 | TRCN0000075558 | Klf3 |
| | 896 | TRCN0000075559 | Klf3 |
| | 897 | TRCN0000075560 | Klf3 |
| | 898 | TRCN0000075561 | Klf3 |
| | 899 | TRCN0000075562 | Klf3 |
| 187 | 900 | TRCN0000034384 | Kras |
| | 901 | TRCN0000034385 | Kras |
| | 902 | TRCN0000034386 | Kras |
| | 903 | TRCN0000034387 | Kras |
| | 904 | TRCN0000034388 | Kras |
| 188 | 905 | TRCN0000022524 | Ksr1 |
| | 906 | TRCN0000022525 | Ksr1 |
| | 907 | TRCN0000022527 | Ksr1 |
| | 908 | TRCN0000022528 | Ksr1 |
| 189 | 909 | TRCN0000022594 | Ksr2 |
| | 910 | TRCN0000022595 | Ksr2 |
| | 911 | TRCN0000022596 | Ksr2 |
| | 912 | TRCN0000022597 | Ksr2 |
| | 913 | TRCN0000022598 | Ksr2 |
| 190 | 914 | TRCN0000075563 | Lasp1 |
| | 915 | TRCN0000075564 | Lasp1 |
| | 916 | TRCN0000075565 | Lasp1 |
| | 917 | TRCN0000075566 | Lasp1 |
| | 918 | TRCN0000075567 | Lasp1 |
| 191 | 919 | TRCN0000022704 | Lats2 |
| | 920 | TRCN0000022705 | Lats2 |
| | 921 | TRCN0000022706 | Lats2 |
| | 922 | TRCN0000022707 | Lats2 |
| | 923 | TRCN0000022708 | Lats2 |
| 192 | 924 | TRCN0000012673 | Lef1 |
| | 925 | TRCN0000012674 | Lef1 |
| | 926 | TRCN0000012675 | Lef1 |
| | 927 | TRCN0000012676 | Lef1 |
| | 928 | TRCN0000012677 | Lef1 |
| 193 | 929 | TRCN0000067908 | Lefty1 |
| | 930 | TRCN0000067909 | Lefty1 |
| | 931 | TRCN0000067911 | Lefty1 |
| | 932 | TRCN0000067912 | Lefty1 |
| 194 | 933 | TRCN0000070533 | Lhx2 |
| | 934 | TRCN0000070534 | Lhx2 |
| | 935 | TRCN0000070535 | Lhx2 |
| | 936 | TRCN0000070536 | Lhx2 |
| | 937 | TRCN0000070537 | Lhx2 |
| 195 | 938 | TRCN0000095669 | Limd1 |
| | 939 | TRCN0000095670 | Limd1 |
| | 940 | TRCN0000095671 | Limd1 |
| | 941 | TRCN0000095672 | Limd1 |
| | 942 | TRCN0000095673 | Limd1 |
| 196 | 943 | TRCN0000084373 | Lmo4 |
| | 944 | TRCN0000084374 | Lmo4 |
| | 945 | TRCN0000084375 | Lmo4 |
| | 946 | TRCN0000084376 | Lmo4 |
| | 947 | TRCN0000084377 | Lmo4 |
| 197 | 948 | TRCN0000070438 | Lmx1a |
| | 949 | TRCN0000070439 | Lmx1a |
| | 950 | TRCN0000070440 | Lmx1a |
| | 951 | TRCN0000070441 | Lmx1a |
| | 952 | TRCN0000070442 | Lmx1a |
| 198 | 953 | TRCN0000119622 | Lrp1 |
| | 954 | TRCN0000119623 | Lrp1 |
| | 955 | TRCN0000119624 | Lrp1 |
| | 956 | TRCN0000119625 | Lrp1 |
| | 957 | TRCN0000119626 | Lrp1 |
| 199 | 958 | TRCN0000119607 | Lrp1b |
| | 959 | TRCN0000119608 | Lrp1b |
| | 960 | TRCN0000119609 | Lrp1b |
| | 961 | TRCN0000119610 | Lrp1b |
| | 962 | TRCN0000119611 | Lrp1b |
| 200 | 963 | TRCN0000119632 | Lrp4 |
| | 964 | TRCN0000119633 | Lrp4 |
| | 965 | TRCN0000119634 | Lrp4 |
| | 966 | TRCN0000119635 | Lrp4 |
| | 967 | TRCN0000119636 | Lrp4 |
| 201 | 968 | TRCN0000109360 | Lrp6 |
| | 969 | TRCN0000109361 | Lrp6 |
| | 970 | TRCN0000109362 | Lrp6 |
| | 971 | TRCN0000109363 | Lrp6 |
| | 972 | TRCN0000109364 | Lrp6 |
| 202 | 973 | TRCN0000108455 | Lrrc4c |
| | 974 | TRCN0000108456 | Lrrc4c |
| | 975 | TRCN0000108457 | Lrrc4c |
| | 976 | TRCN0000108458 | Lrrc4c |
| | 977 | TRCN0000108459 | Lrrc4c |
| 203 | 978 | TRCN0000102225 | Lrrfip1 |
| | 979 | TRCN0000102226 | Lrrfip1 |
| | 980 | TRCN0000102227 | Lrrfip1 |
| | 981 | TRCN0000102229 | Lrrfip1 |
| 204 | 982 | TRCN0000189740 | Ly6g6c |
| | 983 | TRCN0000190117 | Ly6g6c |
| | 984 | TRCN0000193012 | Ly6g6c |
| | 985 | TRCN0000202432 | Ly6g6c |
| 205 | 986 | TRCN0000012608 | Map2k7 |
| | 987 | TRCN0000012609 | Map2k7 |
| | 988 | TRCN0000012610 | Map2k7 |
| | 989 | TRCN0000012611 | Map2k7 |
| | 990 | TRCN0000012612 | Map2k7 |
| 206 | 991 | TRCN0000012763 | Map3k14 |
| | 992 | TRCN0000012764 | Map3k14 |
| | 993 | TRCN0000012765 | Map3k14 |
| | 994 | TRCN0000012766 | Map3k14 |
| | 995 | TRCN0000012767 | Map3k14 |
| 207 | 996 | TRCN0000012758 | Map4k1 |
| | 997 | TRCN0000012759 | Map4k1 |
| | 998 | TRCN0000012761 | Map4k1 |
| | 999 | TRCN0000012762 | Map4k1 |
| 208 | 1000 | TRCN0000055223 | Mapk14 |
| | 1001 | TRCN0000055224 | Mapk14 |
| | 1002 | TRCN0000055225 | Mapk14 |
| | 1003 | TRCN0000055226 | Mapk14 |
| | 1004 | TRCN0000055227 | Mapk14 |
| 209 | 1005 | TRCN0000023184 | Mapk3 |
| | 1006 | TRCN0000023185 | Mapk3 |
| | 1007 | TRCN0000023186 | Mapk3 |
| | 1008 | TRCN0000023187 | Mapk3 |
| | 1009 | TRCN0000023188 | Mapk3 |
| 210 | 1010 | TRCN0000023179 | Mapk4 |
| | 1011 | TRCN0000023180 | Mapk4 |
| | 1012 | TRCN0000023181 | Mapk4 |
| | 1013 | TRCN0000023182 | Mapk4 |
| | 1014 | TRCN0000023183 | Mapk4 |
| 211 | 1015 | TRCN0000023199 | Mapk6 |
| | 1016 | TRCN0000023200 | Mapk6 |
| | 1017 | TRCN0000023201 | Mapk6 |
| | 1018 | TRCN0000023202 | Mapk6 |
| | 1019 | TRCN0000023203 | Mapk6 |
| 212 | 1020 | TRCN0000012599 | Mapk8ip1 |
| | 1021 | TRCN0000012600 | Mapk8ip1 |
| 213 | 1022 | TRCN0000004691 | Mcl1 |
| | 1023 | TRCN0000004692 | Mcl1 |
| | 1024 | TRCN0000004693 | Mcl1 |
| | 1025 | TRCN0000004694 | Mcl1 |
| | 1026 | TRCN0000004695 | Mcl1 |
| 214 | 1027 | TRCN0000012068 | Mef2c |
| | 1028 | TRCN0000012069 | Mef2c |
| | 1029 | TRCN0000012070 | Mef2c |
| | 1030 | TRCN0000012071 | Mef2c |
| | 1031 | TRCN0000012072 | Mef2c |
| 215 | 1032 | TRCN0000012523 | Meis1 |
| | 1033 | TRCN0000012524 | Meis1 |
| | 1034 | TRCN0000012525 | Meis1 |
| | 1035 | TRCN0000012526 | Meis1 |
| | 1036 | TRCN0000012527 | Meis1 |
| 216 | 1037 | TRCN0000022599 | Mlk1 |
| | 1038 | TRCN0000022600 | Mlk1 |
| | 1039 | TRCN0000022601 | Mlk1 |
| | 1040 | TRCN0000022602 | Mlk1 |
| | 1041 | TRCN0000022603 | Mlk1 |
| 217 | 1042 | TRCN0000034424 | Mll1 |
| | 1043 | TRCN0000034428 | Mll1 |
| 218 | 1044 | TRCN0000032834 | Mmp16 |
| | 1045 | TRCN0000032835 | Mmp16 |
| | 1046 | TRCN0000032836 | Mmp16 |
| | 1047 | TRCN0000032837 | Mmp16 |
| | 1048 | TRCN0000032838 | Mmp16 |
| 219 | 1049 | TRCN0000071523 | Morf4l1 |
| | 1050 | TRCN0000071524 | Morf4l1 |
| | 1051 | TRCN0000071525 | Morf4l1 |
| | 1052 | TRCN0000071526 | Morf4l1 |
| | 1053 | TRCN0000071527 | Morf4l1 |
| 195 | 1054 | TRCN0000012663 | Mre11a |
| | 1055 | TRCN0000012664 | Mre11a |
| | 1056 | TRCN0000012665 | Mre11a |
| | 1057 | TRCN0000012667 | Mre11a |
| 196 | 1058 | TRCN0000070623 | Msx1 |
| | 1059 | TRCN0000070624 | Msx1 |
| | 1060 | TRCN0000070625 | Msx1 |
| | 1061 | TRCN0000070626 | Msx1 |
| | 1062 | TRCN0000070627 | Msx1 |
| 197 | 1063 | TRCN0000075943 | Mthfd11 |
| | 1064 | TRCN0000075944 | Mthfd11 |
| | 1065 | TRCN0000075945 | Mthfd11 |
| | 1066 | TRCN0000075946 | Mthfd11 |
| | 1067 | TRCN0000075947 | Mthfd11 |
| 198 | 1068 | TRCN0000042513 | Myc |
| | 1069 | TRCN0000042514 | Myc |
| | 1070 | TRCN0000042515 | Myc |
| | 1071 | TRCN0000042516 | Myc |
| | 1072 | TRCN0000042517 | Myc |
| | 1073 | TRCN0000054853 | Myc |
| | 1074 | TRCN0000054854 | Myc |
| | 1075 | TRCN0000054855 | Myc |
| | 1076 | TRCN0000054856 | Myc |
| 199 | 1077 | TRCN0000011993 | Myef2 |
| | 1078 | TRCN0000011994 | Myef2 |
| | 1079 | TRCN0000011995 | Myef2 |
| | 1080 | TRCN0000011996 | Myef2 |
| | 1081 | TRCN0000011997 | Myef2 |
| 200 | 1082 | TRCN0000071503 | Myh9 |
| | 1083 | TRCN0000071504 | Myh9 |
| | 1084 | TRCN0000071505 | Myh9 |
| | 1085 | TRCN0000071506 | Myh9 |
| | 1086 | TRCN0000071507 | Myh9 |
| 201 | 1087 | TRCN0000125409 | Nav1 |
| | 1088 | TRCN0000125410 | Nav1 |
| | 1089 | TRCN0000125411 | Nav1 |
| | 1090 | TRCN0000125412 | Nav1 |
| | 1091 | TRCN0000125413 | Nav1 |
| 202 | 1092 | TRCN0000009791 | Nedd9 |
| | 1093 | TRCN0000009792 | Nedd9 |
| | 1094 | TRCN0000009793 | Nedd9 |
| | 1095 | TRCN0000009794 | Nedd9 |
| | 1096 | TRCN0000009795 | Nedd9 |
| 203 | 1097 | TRCN0000087559 | Neto1 |
| | 1098 | TRCN0000087560 | Neto1 |
| | 1099 | TRCN0000087561 | Neto1 |
| | 1100 | TRCN0000087562 | Neto1 |
| 204 | 1101 | TRCN0000086943 | Neto2 |
| | 1102 | TRCN0000086944 | Neto2 |
| | 1103 | TRCN0000086945 | Neto2 |
| | 1104 | TRCN0000086946 | Neto2 |
| | 1105 | TRCN0000086947 | Neto2 |
| 205 | 1106 | TRCN0000034339 | Nf1 |
| | 1107 | TRCN0000034340 | Nf1 |
| | 1108 | TRCN0000034341 | Nf1 |
| | 1109 | TRCN0000034342 | Nf1 |
| | 1110 | TRCN0000034343 | Nf1 |
| 206 | 1111 | TRCN0000075343 | Nfe2l1 |
| | 1112 | TRCN0000075344 | Nfe2l1 |
| | 1113 | TRCN0000075345 | Nfe2l1 |
| | 1114 | TRCN0000075346 | Nfe2l1 |
| | 1115 | TRCN0000075347 | Nfe2l1 |
| 207 | 1116 | TRCN0000012128 | Nfe2l2 |
| | 1117 | TRCN0000012129 | Nfe2l2 |
| | 1118 | TRCN0000012130 | Nfe2l2 |
| | 1119 | TRCN0000012131 | Nfe2l2 |
| | 1120 | TRCN0000012132 | Nfe2l2 |
| | 1121 | TRCN0000054658 | Nfe2l2 |
| | 1122 | TRCN0000054659 | Nfe2l2 |
| | 1123 | TRCN0000054660 | Nfe2l2 |
| | 1124 | TRCN0000054661 | Nfe2l2 |
| | 1125 | TRCN0000054662 | Nfe2l2 |
| 208 | 1126 | TRCN0000012088 | Nfib |
| | 1127 | TRCN0000012089 | Nfib |
| | 1128 | TRCN0000012090 | Nfib |
| | 1129 | TRCN0000012091 | Nfib |
| | 1130 | TRCN0000012092 | Nfib |
| 209 | 1131 | TRCN0000075348 | Nfix |
| | 1132 | TRCN0000075349 | Nfix |
| | 1133 | TRCN0000075350 | Nfix |
| | 1134 | TRCN0000075351 | Nfix |
| | 1135 | TRCN0000075352 | Nfix |
| 210 | 1136 | TRCN0000096119 | Nfkbia |
| | 1137 | TRCN0000096120 | Nfkbia |
| | 1138 | TRCN0000096121 | Nfkbia |
| | 1139 | TRCN0000096122 | Nfkbia |
| | 1140 | TRCN0000096123 | Nfkbia |
| 211 | 1141 | TRCN0000025895 | Notch1 |
| | 1142 | TRCN0000025902 | Notch1 |
| | 1143 | TRCN0000025908 | Notch1 |
| | 1144 | TRCN0000025918 | Notch1 |
| | 1145 | TRCN0000025935 | Notch1 |
| 212 | 1146 | TRCN0000012063 | Nr1d2 |
| | 1147 | TRCN0000012064 | Nr1d2 |
| | 1148 | TRCN0000012065 | Nr1d2 |
| | 1149 | TRCN0000012066 | Nr1d2 |
| | 1150 | TRCN0000012067 | Nr1d2 |
| 213 | 1151 | TRCN0000034389 | Nras |
| | 1152 | TRCN0000034390 | Nras |
| | 1153 | TRCN0000034391 | Nras |
| | 1154 | TRCN0000034392 | Nras |
| | 1155 | TRCN0000034393 | Nras |
| 214 | 1156 | TRCN0000025299 | Nrk |
| | 1157 | TRCN0000025300 | Nrk |
| | 1158 | TRCN0000025301 | Nrk |
| | 1159 | TRCN0000025302 | Nrk |
| | 1160 | TRCN0000025303 | Nrk |
| 215 | 1161 | TRCN0000029859 | Nrp1 |
| | 1162 | TRCN0000029860 | Nrp1 |
| | 1163 | TRCN0000029861 | Nrp1 |
| | 1164 | TRCN0000029862 | Nrp1 |
| | 1165 | TRCN0000029863 | Nrp1 |
| 216 | 1166 | TRCN0000028974 | Nrp2 |
| | 1167 | TRCN0000028975 | Nrp2 |
| | 1168 | TRCN0000028976 | Nrp2 |
| | 1169 | TRCN0000028977 | Nrp2 |
| | 1170 | TRCN0000028978 | Nrp2 |
| 217 | 1171 | TRCN0000094624 | Nrxn1 |
| | 1172 | TRCN0000094625 | Nrxn1 |
| | 1173 | TRCN0000094626 | Nrxn1 |
| | 1174 | TRCN0000094627 | Nrxn1 |
| | 1175 | TRCN0000094628 | Nrxn1 |
| 218 | 1176 | TRCN0000094486 | Nrxn2 |
| | 1177 | TRCN0000094487 | Nrxn2 |
| | 1178 | TRCN0000094488 | Nrxn2 |
| 219 | 1179 | TRCN0000094189 | Nrxn3 |
| | 1180 | TRCN0000094190 | Nrxn3 |
| | 1181 | TRCN0000094191 | Nrxn3 |
| | 1182 | TRCN0000094192 | Nrxn3 |
| | 1183 | TRCN0000094193 | Nrxn3 |
| 220 | 1184 | TRCN0000114176 | Nudt14 |
| | 1185 | TRCN0000114177 | Nudt14 |
| | 1186 | TRCN0000114178 | Nudt14 |
| | 1187 | TRCN0000114179 | Nudt14 |
| | 1188 | TRCN0000114180 | Nudt14 |
| 221 | 1189 | TRCN0000072128 | Numa1 |
| | 1190 | TRCN0000072129 | Numa1 |
| | 1191 | TRCN0000072130 | Numa1 |
| | 1192 | TRCN0000072131 | Numa1 |
| 222 | 1193 | TRCN0000075838 | Oas1f |
| | 1194 | TRCN0000075839 | Oas1f |
| | 1195 | TRCN0000075840 | Oas1f |
| | 1196 | TRCN0000075841 | Oas1f |
| | 1197 | TRCN0000075842 | Oas1f |
| 223 | 1198 | TRCN0000071193 | Orc3l |
| | 1199 | TRCN0000071194 | Orc3l |
| | 1200 | TRCN0000071195 | Orc3l |
| | 1201 | TRCN0000071197 | Orc3l |
| 224 | 1202 | TRCN0000025154 | Pak3 |
| | 1203 | TRCN0000025155 | Pak3 |
| | 1204 | TRCN0000025156 | Pak3 |
| | 1205 | TRCN0000025157 | Pak3 |
| | 1206 | TRCN0000025158 | Pak3 |
| 225 | 1207 | TRCN0000032809 | Pappa2 |
| | 1208 | TRCN0000032810 | Pappa2 |
| | 1209 | TRCN0000032811 | Pappa2 |
| | 1210 | TRCN0000032812 | Pappa2 |
| | 1211 | TRCN0000032813 | Pappa2 |
| 226 | 1212 | TRCN0000012573 | Pbx1 |
| | 1213 | TRCN0000012574 | Pbx1 |
| | 1214 | TRCN0000012577 | Pbx1 |
| 227 | 1215 | TRCN0000094899 | Pcdh15 |
| | 1216 | TRCN0000094900 | Pcdh15 |
| | 1217 | TRCN0000094901 | Pcdh15 |
| | 1218 | TRCN0000094902 | Pcdh15 |
| | 1219 | TRCN0000094903 | Pcdh15 |
| 228 | 1220 | TRCN0000111680 | Pclo |
| | 1221 | TRCN0000111681 | Pclo |
| | 1222 | TRCN0000111682 | Pclo |
| | 1223 | TRCN0000111683 | Pclo |
| | 1224 | TRCN0000111684 | Pclo |
| 229 | 1225 | TRCN0000174416 | Pdpn |
| | 1226 | TRCN0000174621 | Pdpn |
| | 1227 | TRCN0000175972 | Pdpn |
| | 1228 | TRCN0000176005 | Pdpn |
| 230 | 1229 | TRCN0000025977 | Pgr |
| | 1230 | TRCN0000025996 | Pgr |
| | 1231 | TRCN0000026003 | Pgr |
| | 1232 | TRCN0000026032 | Pgr |
| 231 | 1233 | TRCN0000055083 | Phlda2 |
| | 1234 | TRCN0000055084 | Phlda2 |
| | 1235 | TRCN0000055085 | Phlda2 |
| | 1236 | TRCN0000055086 | Phlda2 |
| | 1237 | TRCN0000055087 | Phlda2 |
| 232 | 1238 | TRCN0000088628 | Pik3ap1 |
| | 1239 | TRCN0000088629 | Pik3ap1 |
| | 1240 | TRCN0000088630 | Pik3ap1 |
| | 1241 | TRCN0000088631 | Pik3ap1 |
| | 1242 | TRCN0000088632 | Pik3ap1 |
| 233 | 1243 | TRCN0000025614 | Pik3ca |
| | 1244 | TRCN0000025615 | Pik3ca |
| | 1245 | TRCN0000025616 | Pik3ca |
| | 1246 | TRCN0000025617 | Pik3ca |
| | 1247 | TRCN0000025618 | Pik3ca |
| 234 | 1248 | TRCN0000024584 | Pip5k1a |
| | 1249 | TRCN0000024585 | Pip5k1a |
| | 1250 | TRCN0000024586 | Pip5k1a |
| | 1251 | TRCN0000024587 | Pip5k1a |
| | 1252 | TRCN0000024588 | Pip5k1a |
| 235 | 1253 | TRCN0000054653 | Pitx1 |
| | 1254 | TRCN0000054654 | Pitx1 |
| | 1255 | TRCN0000054655 | Pitx1 |
| | 1256 | TRCN0000054656 | Pitx1 |
| | 1257 | TRCN0000054657 | Pitx1 |
| 236 | 1258 | TRCN0000072083 | Pkd1 |
| | 1259 | TRCN0000072084 | Pkd1 |
| | 1260 | TRCN0000072085 | Pkd1 |
| | 1261 | TRCN0000072086 | Pkd1 |
| | 1262 | TRCN0000072087 | Pkd1 |
| 237 | 1263 | TRCN0000123359 | Pkp4 |
| | 1264 | TRCN0000123360 | Pkp4 |
| | 1265 | TRCN0000123361 | Pkp4 |
| | 1266 | TRCN0000123362 | Pkp4 |
| | 1267 | TRCN0000123363 | Pkp4 |
| 238 | 1268 | TRCN0000076908 | Plcb1 |
| | 1269 | TRCN0000076909 | Plcb1 |
| | 1270 | TRCN0000076910 | Plcb1 |
| | 1271 | TRCN0000076911 | Plcb1 |
| | 1272 | TRCN0000076912 | Plcb1 |
| 239 | 1273 | TRCN0000105980 | Ppp1r9a |
| | 1274 | TRCN0000105981 | Ppp1r9a |
| | 1275 | TRCN0000105982 | Ppp1r9a |
| | 1276 | TRCN0000105983 | Ppp1r9a |
| | 1277 | TRCN0000105984 | Ppp1r9a |
| 240 | 1278 | TRCN0000081058 | Ppp3ca |
| | 1279 | TRCN0000081059 | Ppp3ca |
| | 1280 | TRCN0000081060 | Ppp3ca |
| | 1281 | TRCN0000081061 | Ppp3ca |
| | 1282 | TRCN0000081062 | Ppp3ca |
| 241 | 1283 | TRCN0000085193 | Prdm9 |
| | 1284 | TRCN0000085194 | Prdm9 |
| | 1285 | TRCN0000085195 | Prdm9 |
| | 1286 | TRCN0000085196 | Prdm9 |
| | 1287 | TRCN0000085197 | Prdm9 |
| 242 | 1288 | TRCN0000091048 | Prickle2 |
| | 1289 | TRCN0000091049 | Prickle2 |
| | 1290 | TRCN0000091050 | Prickle2 |
| | 1291 | TRCN0000091051 | Prickle2 |
| | 1292 | TRCN0000091052 | Prickle2 |
| 243 | 1293 | TRCN0000022875 | Prkca |
| | 1294 | TRCN0000022878 | Prkca |
| | 1295 | TRCN0000022754 | Prkci |
| 244 | 1296 | TRCN0000022755 | Prkci |
| | 1297 | TRCN0000022756 | Prkci |
| | 1298 | TRCN0000022757 | Prkci |
| | 1299 | TRCN0000022758 | Prkci |
| 245 | 1300 | TRCN0000022717 | Prkg2 |
| | 1301 | TRCN0000022718 | Prkg2 |
| 246 | 1302 | TRCN0000115318 | Prom1 (CD133) |
| | 1303 | TRCN0000115316 | Prom1 (CD133) |
| | 1304 | TRCN0000115317 | Prom1 (CD133) |
| | 1305 | TRCN0000115319 | Prom1 (CD133) |
| | 1306 | TRCN0000115320 | Prom1 (CD133) |
| 247 | 1307 | TRCN0000025359 | Prpf4b |
| | 1308 | TRCN0000025360 | Prpf4b |
| | 1309 | TRCN0000025361 | Prpf4b |
| | 1310 | TRCN0000025362 | Prpf4b |
| | 1311 | TRCN0000025363 | Prpf4b |
| 248 | 1312 | TRCN0000012113 | Psip1 |
| | 1313 | TRCN0000012114 | Psip1 |
| | 1314 | TRCN0000012115 | Psip1 |
| | 1315 | TRCN0000012116 | Psip1 |
| | 1316 | TRCN0000012117 | Psip1 |
| 249 | 1317 | TRCN0000042538 | Ptch1 |
| | 1318 | TRCN0000042539 | Ptch1 |
| | 1319 | TRCN0000042540 | Ptch1 |
| | 1320 | TRCN0000042541 | Ptch1 |
| | 1321 | TRCN0000042542 | Ptch1 |
| 250 | 1322 | TRCN0000028989 | Pten |
| | 1323 | TRCN0000028991 | Pten |
| | 1324 | TRCN0000028993 | Pten |
| 251 | 1325 | TRCN0000011913 | Ptgds |
| | 1326 | TRCN0000011914 | Ptgds |
| | 1327 | TRCN0000011915 | Ptgds |
| | 1328 | TRCN0000011916 | Ptgds |
| | 1329 | TRCN0000011917 | Ptgds |
| 252 | 1330 | TRCN0000067938 | Ptgs2 |
| | 1331 | TRCN0000067939 | Ptgs2 |
| | 1332 | TRCN0000067940 | Ptgs2 |
| | 1333 | TRCN0000067941 | Ptgs2 |
| | 1334 | TRCN0000067942 | Ptgs2 |
| 253 | 1335 | TRCN0000023484 | Ptk2 |
| | 1336 | TRCN0000023485 | Ptk2 |
| | 1337 | TRCN0000023486 | Ptk2 |
| | 1338 | TRCN0000023487 | Ptk2 |
| | 1339 | TRCN0000023488 | Ptk2 |
| 254 | 1340 | TRCN0000081068 | Ptprz1 |
| | 1341 | TRCN0000081069 | Ptprz1 |
| | 1342 | TRCN0000081070 | Ptprz1 |
| | 1343 | TRCN0000081071 | Ptprz1 |
| | 1344 | TRCN0000081072 | Ptprz1 |
| 255 | 1345 | TRCN0000100435 | Rab31 |
| | 1346 | TRCN0000100436 | Rab31 |
| | 1347 | TRCN0000100437 | Rab31 |
| | 1348 | TRCN0000100438 | Rab31 |
| | 1349 | TRCN0000100439 | Rab31 |
| 256 | 1350 | TRCN0000055188 | Rac1 |
| | 1351 | TRCN0000055189 | Rac1 |
| | 1352 | TRCN0000055190 | Rac1 |
| | 1353 | TRCN0000055191 | Rac1 |
| | 1354 | TRCN0000055192 | Rac1 |
| 257 | 1355 | TRCN0000012658 | Rad51 |
| | 1356 | TRCN0000012659 | Rad51 |
| | 1357 | TRCN0000012660 | Rad51 |
| | 1358 | TRCN0000012661 | Rad51 |
| | 1359 | TRCN0000012662 | Rad51 |
| 258 | 1360 | TRCN0000012628 | Raf1 |
| | 1361 | TRCN0000012629 | Raf1 |
| | 1362 | TRCN0000012630 | Raf1 |
| | 1363 | TRCN0000012631 | Raf1 |
| | 1364 | TRCN0000012632 | Raf1 |
| | 1365 | TRCN0000055138 | Raf1 |
| | 1366 | TRCN0000055139 | Raf1 |
| | 1367 | TRCN0000055140 | Raf1 |
| | 1368 | TRCN0000055141 | Raf1 |
| | 1369 | TRCN0000055142 | Raf1 |
| 259 | 1370 | TRCN0000071953 | Rapgef3 |
| | 1371 | TRCN0000071954 | Rapgef3 |
| | 1372 | TRCN0000071955 | Rapgef3 |
| | 1373 | TRCN0000071956 | Rapgef3 |
| | 1374 | TRCN0000071957 | Rapgef3 |
| | 1375 | TRCN0000077653 | Rasa1 |
| | 1376 | TRCN0000077654 | Rasa1 |
| | 1377 | TRCN0000077655 | Rasa1 |
| | 1378 | TRCN0000077656 | Rasa1 |
| | 1379 | TRCN0000077657 | Rasa1 |
| 260 | 1380 | TRCN0000042543 | Rb1 |
| | 1381 | TRCN0000042544 | Rb1 |
| | 1382 | TRCN0000042545 | Rb1 |
| | 1383 | TRCN0000042546 | Rb1 |
| | 1384 | TRCN0000042547 | Rb1 |
| | 1385 | TRCN0000055378 | Rb1 |
| | 1386 | TRCN0000055379 | Rb1 |
| | 1387 | TRCN0000055380 | Rb1 |
| | 1388 | TRCN0000055381 | Rb1 |
| | 1389 | TRCN0000055382 | Rb1 |
| 261 | 1390 | TRCN0000071273 | Rbl2 |
| | 1391 | TRCN0000071274 | Rbl2 |
| | 1392 | TRCN0000071275 | Rbl2 |
| | 1393 | TRCN0000071276 | Rbl2 |
| | 1394 | TRCN0000071277 | Rbl2 |
| 262 | 1395 | TRCN0000042548 | Rel |
| | 1396 | TRCN0000042549 | Rel |
| | 1397 | TRCN0000042550 | Rel |
| | 1398 | TRCN0000042551 | Rel |
| | 1399 | TRCN0000042552 | Rel |
| 263 | 1400 | TRCN0000120627 | Reln |
| | 1401 | TRCN0000120628 | Reln |
| | 1402 | TRCN0000120629 | Reln |
| | 1403 | TRCN0000120630 | Reln |
| | 1404 | TRCN0000120631 | Reln |
| 264 | 1405 | TRCN0000071343 | Rest |
| | 1406 | TRCN0000071344 | Rest |
| | 1407 | TRCN0000071345 | Rest |
| | 1408 | TRCN0000071346 | Rest |
| | 1409 | TRCN0000071347 | Rest |
| 265 | 1410 | TRCN0000106155 | Rims2 |
| | 1411 | TRCN0000106156 | Rims2 |
| | 1412 | TRCN0000106157 | Rims2 |
| | 1413 | TRCN0000106158 | Rims2 |
| | 1414 | TRCN0000106159 | Rims2 |
| 266 | 1415 | TRCN0000022634 | Ripk4 |
| | 1416 | TRCN0000022635 | Ripk4 |
| | 1417 | TRCN0000022636 | Ripk4 |
| | 1418 | TRCN0000022637 | Ripk4 |
| | 1419 | TRCN0000022638 | Ripk4 |
| 267 | 1420 | TRCN0000027509 | Rxfp3 |
| | 1421 | TRCN0000027517 | Rxfp3 |
| | 1422 | TRCN0000027523 | Rxfp3 |
| | 1423 | TRCN0000027528 | Rxfp3 |
| | 1424 | TRCN0000027574 | Rxfp3 |
| 268 | 1425 | TRCN0000011858 | S100a4 |
| | 1426 | TRCN0000011859 | S100a4 |
| | 1427 | TRCN0000011860 | S100a4 |
| | 1428 | TRCN0000011861 | S100a4 |
| | 1429 | TRCN0000011862 | S100a4 |
| 269 | 1430 | TRCN0000072043 | S100a9 |
| | 1431 | TRCN0000072044 | S100a9 |
| | 1432 | TRCN0000072045 | S100a9 |
| | 1433 | TRCN0000072046 | S100a9 |
| | 1434 | TRCN0000072047 | S100a9 |
| 270 | 1435 | TRCN0000071628 | Sfrs3 |
| | 1436 | TRCN0000071629 | Sfrs3 |
| | 1437 | TRCN0000071630 | Sfrs3 |
| | 1438 | TRCN0000071631 | Sfrs3 |
| | 1439 | TRCN0000071632 | Sfrs3 |
| 271 | 1440 | TRCN0000071933 | Sfrs7 |
| | 1441 | TRCN0000071934 | Sfrs7 |
| | 1442 | TRCN0000071935 | Sfrs7 |
| | 1443 | TRCN0000071936 | Sfrs7 |
| | 1444 | TRCN0000071937 | Sfrs7 |
| 272 | 1445 | TRCN0000022884 | Sgk |
| | 1446 | TRCN0000022885 | Sgk |
| | 1447 | TRCN0000022886 | Sgk |
| | 1448 | TRCN0000022887 | Sgk |
| 273 | 1449 | TRCN0000022879 | Sgk2 |
| | 1450 | TRCN0000022880 | Sgk2 |
| | 1451 | TRCN0000022881 | Sgk2 |
| | 1452 | TRCN0000022882 | Sgk2 |
| | 1453 | TRCN0000022883 | Sgk2 |
| 274 | 1454 | TRCN0000011953 | Si |
| | 1455 | TRCN0000011954 | Si |
| | 1456 | TRCN0000011955 | Si |
| | 1457 | TRCN0000011956 | Si |
| | 1458 | TRCN0000011957 | Si |
| 275 | 1459 | TRCN0000042563 | Ski |
| | 1460 | TRCN0000042564 | Ski |
| | 1461 | TRCN0000042565 | Ski |
| | 1462 | TRCN0000042566 | Ski |
| | 1463 | TRCN0000042567 | Ski |
| 276 | 1464 | TRCN0000079543 | Slc16a1 |
| | 1465 | TRCN0000079544 | Slc16a1 |
| | 1466 | TRCN0000079545 | Slc16a1 |
| | 1467 | TRCN0000079546 | Slc16a1 |
| | 1468 | TRCN0000079547 | Slc16a1 |
| 277 | 1469 | TRCN0000079308 | Slc6a2 |
| | 1470 | TRCN0000079309 | Slc6a2 |
| | 1471 | TRCN0000079310 | Slc6a2 |
| | 1472 | TRCN0000079311 | Slc6a2 |
| | 1473 | TRCN0000079312 | Slc6a2 |
| 278 | 1474 | TRCN0000079253 | Slco3a1 |
| | 1475 | TRCN0000079254 | Slco3a1 |
| | 1476 | TRCN0000079255 | Slco3a1 |
| | 1477 | TRCN0000079256 | Slco3a1 |
| | 1478 | TRCN0000079257 | Slco3a1 |
| 279 | 1479 | TRCN0000106575 | Slit1 |
| | 1480 | TRCN0000106576 | Slit1 |
| | 1481 | TRCN0000106577 | Slit1 |
| | 1482 | TRCN0000106578 | Slit1 |
| | 1483 | TRCN0000106579 | Slit1 |
| 280 | 1484 | TRCN0000120817 | Slit2 |
| | 1485 | TRCN0000120818 | Slit2 |
| | 1486 | TRCN0000120819 | Slit2 |
| | 1487 | TRCN0000120820 | Slit2 |
| | 1488 | TRCN0000120821 | Slit2 |
| 281 | 1489 | TRCN0000114071 | Slitrk3 |
| | 1490 | TRCN0000114073 | Slitrk3 |
| | 1491 | TRCN0000114074 | Slitrk3 |
| | 1492 | TRCN0000114075 | Slitrk3 |
| 282 | 1493 | TRCN0000025884 | Smad1 |
| | 1494 | TRCN0000025910 | Smad1 |
| | 1495 | TRCN0000025933 | Smad1 |
| | 1496 | TRCN0000025963 | Smad1 |
| 283 | 1497 | TRCN0000025881 | Smad4 |
| | 1498 | TRCN0000025885 | Smad4 |
| | 1499 | TRCN0000025900 | Smad4 |
| | 1500 | TRCN0000025953 | Smad4 |
| 284 | 1501 | TRCN0000025891 | Smad9 |
| | 1502 | TRCN0000025893 | Smad9 |
| | 1503 | TRCN0000025912 | Smad9 |
| | 1504 | TRCN0000025913 | Smad9 |
| | 1505 | TRCN0000025937 | Smad9 |
| 285 | 1506 | TRCN0000071398 | Smarca2 |
| | 1507 | TRCN0000071399 | Smarca2 |
| | 1508 | TRCN0000071400 | Smarca2 |
| | 1509 | TRCN0000071401 | Smarca2 |
| | 1510 | TRCN0000071402 | Smarca2 |
| 286 | 1511 | TRCN0000085748 | Sox2 |
| | 1512 | TRCN0000085749 | Sox2 |
| | 1513 | TRCN0000085750 | Sox2 |
| | 1514 | TRCN0000085751 | Sox2 |
| | 1515 | TRCN0000085752 | Sox2 |
| 287 | 1516 | TRCN0000086338 | Spic |
| | 1517 | TRCN0000086339 | Spic |
| | 1518 | TRCN0000086340 | Spic |
| | 1519 | TRCN0000086341 | Spic |
| | 1520 | TRCN0000086342 | Spic |
| 288 | 1521 | TRCN0000087743 | Spink5 |
| | 1522 | TRCN0000087744 | Spink5 |
| | 1523 | TRCN0000087745 | Spink5 |
| | 1524 | TRCN0000087746 | Spink5 |
| | 1525 | TRCN0000087747 | Spink5 |
| 289 | 1526 | TRCN0000009601 | Spp1 |
| | 1527 | TRCN0000009602 | Spp1 |
| | 1528 | TRCN0000009603 | Spp1 |
| | 1529 | TRCN0000009604 | Spp1 |
| | 1530 | TRCN0000009605 | Spp1 |
| | 1531 | TRCN0000054698 | Spp1 |
| | 1532 | TRCN0000054699 | Spp1 |
| | 1533 | TRCN0000054700 | Spp1 |
| | 1534 | TRCN0000054701 | Spp1 |
| | 1535 | TRCN0000054702 | Spp1 |
| 290 | 1536 | TRCN0000098415 | Sprr1b |
| | 1537 | TRCN0000098416 | Sprr1b |
| | 1538 | TRCN0000098417 | Sprr1b |
| | 1539 | TRCN0000098418 | Sprr1b |
| | 1540 | TRCN0000098419 | Sprr1b |
| 301 | 1541 | TRCN0000065478 | Spry1 |
| | 1542 | TRCN0000065479 | Spry1 |
| | 1543 | TRCN0000065480 | Spry1 |
| | 1544 | TRCN0000065481 | Spry1 |
| | 1545 | TRCN0000065482 | Spry1 |
| 302 | 1546 | TRCN0000103591 | Spry2 |
| | 1547 | TRCN0000103592 | Spry2 |
| | 1548 | TRCN0000103593 | Spry2 |
| | 1549 | TRCN0000103594 | Spry2 |
| 303 | 1550 | TRCN0000065538 | Spry3 |
| | 1551 | TRCN0000065539 | Spry3 |
| | 1552 | TRCN0000065540 | Spry3 |
| | 1553 | TRCN0000065541 | Spry3 |
| | 1554 | TRCN0000065542 | Spry3 |
| 304 | 1555 | TRCN0000065934 | Spry4 |
| | 1556 | TRCN0000065935 | Spry4 |
| | 1557 | TRCN0000065936 | Spry4 |
| | 1558 | TRCN0000065937 | Spry4 |
| 305 | 1559 | TRCN0000103170 | Sptlc2 |
| | 1560 | TRCN0000103171 | Sptlc2 |
| | 1561 | TRCN0000103172 | Sptlc2 |
| | 1562 | TRCN0000103173 | Sptlc2 |
| | 1563 | TRCN0000103174 | Sptlc2 |
| 306 | 1564 | TRCN0000125734 | Steap1 |
| | 1565 | TRCN0000125735 | Steap1 |
| | 1566 | TRCN0000125736 | Steap1 |
| | 1567 | TRCN0000125737 | Steap1 |
| | 1568 | TRCN0000125738 | Steap1 |
| 307 | 1569 | TRCN0000023729 | Styk1 |
| | 1570 | TRCN0000023730 | Styk1 |
| | 1571 | TRCN0000023731 | Styk1 |
| | 1572 | TRCN0000023732 | Styk1 |
| | 1573 | TRCN0000023733 | Styk1 |
| 308 | 1574 | TRCN0000072048 | Sub1 |
| | 1575 | TRCN0000072049 | Sub1 |
| | 1576 | TRCN0000072050 | Sub1 |
| | 1577 | TRCN0000072051 | Sub1 |
| | 1578 | TRCN0000072052 | Sub1 |
| 309 | 1579 | TRCN0000125999 | Susd2 |
| | 1580 | TRCN0000126000 | Susd2 |
| | 1581 | TRCN0000126001 | Susd2 |
| | 1582 | TRCN0000126002 | Susd2 |
| | 1583 | TRCN0000126003 | Susd2 |
| 310 | 1584 | TRCN0000108875 | Syne1 |
| | 1585 | TRCN0000108876 | Syne1 |
| | 1586 | TRCN0000108877 | Syne1 |
| | 1587 | TRCN0000108878 | Syne1 |
| | 1588 | TRCN0000108879 | Syne1 |
| 311 | 1589 | TRCN0000042573 | Tal1 |
| | 1590 | TRCN0000042574 | Tal1 |
| | 1591 | TRCN0000042575 | Tal1 |
| | 1592 | TRCN0000042576 | Tal1 |
| | 1593 | TRCN0000042577 | Tal1 |
| 312 | 1594 | TRCN0000176581 | Tanc1 |
| | 1595 | TRCN0000176582 | Tanc1 |
| | 1596 | TRCN0000178012 | Tanc1 |
| | 1597 | TRCN0000178631 | Tanc1 |
| 313 | 1598 | TRCN0000012093 | Tcf4 |
| | 1599 | TRCN0000012094 | Tcf4 |
| | 1600 | TRCN0000012095 | Tcf4 |
| | 1601 | TRCN0000012096 | Tcf4 |
| | 1602 | TRCN0000012097 | Tcf4 |
| 314 | 1603 | TRCN0000012178 | Tcf7l2 |
| | 1604 | TRCN0000012179 | Tcf7l2 |
| | 1605 | TRCN0000012180 | Tcf7l2 |
| | 1606 | TRCN0000012181 | Tcf7l2 |
| 315 | 1607 | TRCN0000075508 | Tcfap2c |
| | 1608 | TRCN0000075509 | Tcfap2c |
| | 1609 | TRCN0000075510 | Tcfap2c |
| | 1610 | TRCN0000075511 | Tcfap2c |
| | 1611 | TRCN0000075512 | Tcfap2c |
| 316 | 1612 | TRCN0000086223 | Tcfap2e |
| | 1613 | TRCN0000086224 | Tcfap2e |
| | 1614 | TRCN0000086225 | Tcfap2e |
| | 1615 | TRCN0000086227 | Tcfap2e |
| 317 | 1616 | TRCN0000071308 | Terf2ip |
| | 1617 | TRCN0000071309 | Terf2ip |
| | 1618 | TRCN0000071310 | Terf2ip |
| | 1619 | TRCN0000071311 | Terf2ip |
| | 1620 | TRCN0000071312 | Terf2ip |
| 318 | 1621 | TRCN0000054809 | Tgfbi |
| | 1622 | TRCN0000054811 | Tgfbi |
| 319 | 1623 | TRCN0000022624 | Tgfbr2 |
| | 1624 | TRCN0000022625 | Tgfbr2 |
| | 1625 | TRCN0000022626 | Tgfbr2 |
| | 1626 | TRCN0000022627 | Tgfbr2 |
| | 1627 | TRCN0000022628 | Tgfbr2 |
| 320 | 1628 | TRCN0000075523 | Tgif2 |
| | 1629 | TRCN0000075524 | Tgif2 |
| | 1630 | TRCN0000075525 | Tgif2 |
| | 1631 | TRCN0000075526 | Tgif2 |
| | 1632 | TRCN0000075527 | Tgif2 |
| 321 | 1633 | TRCN0000042593 | Tiam1 |
| | 1634 | TRCN0000042595 | Tiam1 |
| | 1635 | TRCN0000042596 | Tiam1 |
| | 1636 | TRCN0000042597 | Tiam1 |
| 322 | 1637 | TRCN0000112785 | Tm4sf1 |
| | 1638 | TRCN0000112786 | Tm4sf1 |
| | 1639 | TRCN0000112787 | Tm4sf1 |
| | 1640 | TRCN0000112788 | Tm4sf1 |
| | 1641 | TRCN0000112789 | Tm4sf1 |
| 323 | 1642 | TRCN0000174268 | Tm7sf3 |
| | 1643 | TRCN0000174778 | Tm7sf3 |
| | 1644 | TRCN0000193418 | Tm7sf3 |
| | 1645 | TRCN0000193467 | Tm7sf3 |
| | 1646 | TRCN0000193517 | Tm7sf3 |
| 324 | 1647 | TRCN0000110735 | Tnc |
| | 1648 | TRCN0000110736 | Tnc |
| | 1649 | TRCN0000110737 | Tnc |
| | 1650 | TRCN0000110738 | Tnc |
| | 1651 | TRCN0000110739 | Tnc |
| 325 | 1652 | TRCN0000023749 | Tnk2 |
| | 1653 | TRCN0000023750 | Tnk2 |
| | 1654 | TRCN0000023751 | Tnk2 |
| | 1655 | TRCN0000023752 | Tnk2 |
| | 1656 | TRCN0000023753 | Tnk2 |
| 326 | 1657 | TRCN0000070163 | Tnpo2 |
| | 1658 | TRCN0000070164 | Tnpo2 |
| | 1659 | TRCN0000070165 | Tnpo2 |
| | 1660 | TRCN0000070166 | Tnpo2 |
| | 1661 | TRCN0000070167 | Tnpo2 |
| 327 | 1662 | TRCN0000012362 | Trp53 |
| | 1663 | TRCN0000012362 | Trp53 |
| | 1664 | TRCN0000054551 | Trp53 |
| | 1665 | TRCN0000054552 | Trp53 |
| 328 | 1666 | TRCN0000012748 | Trp63 |
| | 1667 | TRCN0000012749 | Trp63 |
| | 1668 | TRCN0000012750 | Trp63 |
| | 1669 | TRCN0000012751 | Trp63 |
| | 1670 | TRCN0000012752 | Trp63 |
| 329 | 1671 | TRCN0000012753 | Trp73 |
| | 1672 | TRCN0000012754 | Trp73 |
| | 1673 | TRCN0000012755 | Trp73 |
| | 1674 | TRCN0000012756 | Trp73 |
| | 1675 | TRCN0000012757 | Trp73 |
| 330 | 1676 | TRCN0000094629 | Tspan6 |
| | 1677 | TRCN0000094630 | Tspan6 |
| | 1678 | TRCN0000094631 | Tspan6 |
| | 1679 | TRCN0000094632 | Tspan6 |
| | 1680 | TRCN0000094633 | Tspan6 |
| 331 | 1681 | TRCN0000094474 | Tspan8 |
| | 1682 | TRCN0000094475 | Tspan8 |
| | 1683 | TRCN0000094477 | Tspan8 |
| | 1684 | TRCN0000094478 | Tspan8 |
| 332 | 1685 | TRCN0000088743 | Ttn |
| | 1686 | TRCN0000088744 | Ttn |
| | 1687 | TRCN0000088745 | Ttn |
| | 1688 | TRCN0000088746 | Ttn |
| | 1689 | TRCN0000088747 | Ttn |
| 333 | 1690 | TRCN0000071573 | Usf2 |
| | 1691 | TRCN0000071574 | Usf2 |
| | 1692 | TRCN0000071575 | Usf2 |
| | 1693 | TRCN0000071576 | Usf2 |
| | 1694 | TRCN0000071577 | Usf2 |
| 334 | 1695 | TRCN0000042608 | Vav1 |
| | 1696 | TRCN0000042609 | Vav1 |
| | 1697 | TRCN0000042610 | Vav1 |
| | 1698 | TRCN0000042611 | Vav1 |
| | 1699 | TRCN0000042612 | Vav1 |
| 335 | 1700 | TRCN0000027068 | Vdr |
| | 1701 | TRCN0000027098 | Vdr |
| | 1702 | TRCN0000027101 | Vdr |
| | 1703 | TRCN0000027104 | Vdr |
| | 1704 | TRCN0000027123 | Vdr |
| 336 | 1705 | TRCN0000066818 | Vegfa |
| | 1706 | TRCN0000066819 | Vegfa |
| | 1707 | TRCN0000066820 | Vegfa |
| | 1708 | TRCN0000066821 | Vegfa |
| | 1709 | TRCN0000066822 | Vegfa |
| 337 | 1710 | TRCN0000097084 | Was |
| | 1711 | TRCN0000097085 | Was |
| | 1712 | TRCN0000097086 | Was |
| | 1713 | TRCN0000097087 | Was |
| | 1714 | TRCN0000097088 | Was |
| 338 | 1715 | TRCN0000012403 | Wasf1 |
| | 1716 | TRCN0000012404 | Wasf1 |
| | 1717 | TRCN0000012405 | Wasf1 |
| | 1718 | TRCN0000012406 | Wasf1 |
| | 1719 | TRCN0000012407 | Wasf1 |
| 339 | 1720 | TRCN0000099640 | Wasl |
| | 1721 | TRCN0000099641 | Wasl |
| | 1722 | TRCN0000099642 | Wasl |
| | 1723 | TRCN0000099643 | Wasl |
| | 1724 | TRCN0000099644 | Wasl |
| 340 | 1725 | TRCN0000183172 | Waspip |
| | 1726 | TRCN0000183384 | Waspip |
| | 1727 | TRCN0000184459 | Waspip |
| | 1728 | TRCN0000195856 | Waspip |
| 341 | 1729 | TRCN0000115481 | Wdr63 |
| | 1730 | TRCN0000115482 | Wdr63 |
| | 1731 | TRCN0000115483 | Wdr63 |
| | 1732 | TRCN0000115484 | Wdr63 |
| | 1733 | TRCN0000115485 | Wdr63 |
| 342 | 1734 | TRCN0000080203 | Wfdc1 |
| | 1735 | TRCN0000080204 | Wfdc1 |
| | 1736 | TRCN0000080205 | Wfdc1 |
| | 1737 | TRCN0000080206 | Wfdc1 |
| | 1738 | TRCN0000080207 | Wfdc1 |
| 343 | 1739 | TRCN0000080198 | Wfdc2 |
| | 1740 | TRCN0000080199 | Wfdc2 |
| | 1741 | TRCN0000080200 | Wfdc2 |
| | 1742 | TRCN0000080201 | Wfdc2 |
| | 1743 | TRCN0000080202 | Wfdc2 |
| 344 | 1744 | TRCN0000042113 | Wwox |
| | 1745 | TRCN0000042114 | Wwox |
| | 1746 | TRCN0000042115 | Wwox |
| | 1747 | TRCN0000042116 | Wwox |
| | 1748 | TRCN0000042117 | Wwox |
| 345 | 1749 | TRCN0000095864 | Yap1 |
| | 1750 | TRCN0000095865 | Yap1 |
| | 1751 | TRCN0000095866 | Yap1 |
| | 1752 | TRCN0000095867 | Yap1 |
| | 1753 | TRCN0000095868 | Yap1 |
| 346 | 1754 | TRCN0000071943 | Zfp503 |
| | 1755 | TRCN0000071944 | Zfp503 |
| | 1756 | TRCN0000071945 | Zfp503 |
| | 1757 | TRCN0000071946 | Zfp503 |
| | 1758 | TRCN0000071947 | Zfp503 |
| 347 | 1759 | TRCN0000096684 | Zic1 |
| | 1760 | TRCN0000096685 | Zic1 |
| | 1761 | TRCN0000096686 | Zic1 |
| | 1762 | TRCN0000096687 | Zic1 |
| | 1763 | TRCN0000096688 | Zic1 |

TABLE 2
List of genes mutated in 306 HNSCC patients ranked by statistical
significance of enrichment of these genes with predicted functional mutations. Number of
genes displayed: 16.

| Gene | Cytoband | TS/OG | CG | Samples | MM | TM | SM | FIS ≧ 2.0 | P val (FIS ≧ 2.0) | Q val (FIS ≧ 2.0) |
|---|---|---|---|---|---|---|---|---|---|---|
| TP53 | 17p13.1 | 1 | 9 | 302 | 171 | 128 | 6 | 160 | 0 | 0 |
| NOTCH1 | 9q34.3 | 1 | 10 | 302 | 43 | 31 | 7 | 33 | 0 | 0 |
| DNAH5 | 5p15.2 | 0 | 0 | 302 | 48 | 14 | 20 | 32 | 0 | 0 |
| NFE2L2 | 2q31.2 | 0 | 2 | 302 | 24 | 0 | 0 | 24 | 0 | 0 |
| CASP8 | 2q33.1 | 1 | 4 | 302 | 15 | 18 | 0 | 12 | 0 | 0 |
| MYH8 | 17p13.1 | 0 | 0 | 302 | 21 | 2 | 4 | 15 | 0.000001 | 0.002 |
| SMARCA4 | 19p13.2 | 1 | 4 | 302 | 16 | 1 | 1 | 12 | 0.000003 | 0.006 |
| FAT1 | 4q35.2 | 1 | 1 | 302 | 22 | 89 | 2 | 13 | 0.000006 | 0.009 |
| RAC1 | 7p22.1 | 0 | 4 | 302 | 10 | 0 | 0 | 9 | 0.000006 | 0.011 |
| CUL3 | 2q36.2 | 0 | 0 | 302 | 9 | 5 | 1 | 8 | 0.000006 | 0.011 |
| HIST1H2BD | 6p22.1 | 0 | 0 | 302 | 6 | 1 | 0 | 6 | 0.000009 | 0.012 |
| SCN3A | 2q24.3 | 0 | 1 | 302 | 16 | 2 | 3 | 13 | 0.00001 | 0.016 |
| PCDHGA1 | 5q31.3 | 0 | 0 | 302 | 10 | 2 | 1 | 9 | 0.00002 | 0.023 |
| PRPF6 | 20q13.33 | 0 | 1 | 302 | 9 | 0 | 2 | 8 | 0.00002 | 0.023 |
| EP300 | 22q13.2 | 0 | 10 | 302 | 22 | 8 | 1 | 14 | 0.00002 | 0.023 |
| MYH9 | 22q12.3 | 0 | 5 | 302 | 16 | 2 | 4 | 12 | 0.00003 | 0.024 |

MM is a number of missense mutations
TM is a number of truncating mutations
SM is a number of silent mutations
FIS ≧ 2 is a number of missense mutations with the predicted functional score bigger than 2 [PMID: 21727090 PMCID: PMC3177186]
DD and D are, respectively, numbers of homozygous and hemizygous deletions
AA and A are, respectively, numbers DNA copy amplifications and DNA copy gains;
P-val (FM) and P-val (FTM) are, respectively, probabilities to observe the obtained distributions of predicted functional mutations and predicted functional and truncating mutations by chance.

TABLE 3
Statistics of genomic alterations of MYH9 across 10 cancer types
found in the TCGA data set.

| | | | | | | | | | | | | Cancers | Cancers | Cancers |
|---|---|---|---|---|---|---|---|---|---|---|---|---|---|---|
| | Cyto- | | Sam- | | | | FIS >= | | | | | with DFTM | with FM | with FTM |
| Gene | band | Cancer type | ples | MM | TM | SM | 2.0 | DD | D | AA | A | enrichment | enrichment | enrichment |
| MYH9 | 22q12.3 | BLCA/ | 3081 | 102 | 24 | 28 | 58 | 5 | 1076 | 16 | 323 | LUSC | LUSC/ | LUSC/ |
| | | LUSC/GBM/ | | | | | | | | | | | COADREAD/ | COADREAD/ |
| | | KIRC/ | | | | | | | | | | | UCEC/ | UCEC/ |
| | | COADREAD/ | | | | | | | | | | | HNSC | HNSC/ |
| | | UCEC/ | | | | | | | | | | | | BRCA/ |
| | | HNSC/ | | | | | | | | | | | | LUAD |
| | | BRCA/OVC/ | | | | | | | | | | | | |
| | | LUAD | | | | | | | | | | | | |

Abbreviations are as in Table 2 and:
BLCA: bladder carcinoma;
LUSC: lung squamous cell carcinoma;
GBM: gliobastoma;
KIRC: Kidney Renal Papillary Cell Carcinoma;
COADREAD: colorectal carcinoma;
UCEC: cervical SCC & endocervical carcinoma;
HNSCC: head and neck SCC;
BRCA: breast carcinoma;
OVC: ovarian carcinoma;
LUAD; lung adenocarcinoma

TABLE 4
List of 18.014 genes mutated in 306 HNSCC patients ranked
according to their p-value and false discovery rate analysed by MutSig2.0 and MutSigCV0.9.
Number of significant genes found: 35. Number of genes displayed: 50.

| rank | gene | description | n | npat | nsite | nsil | p_cons | p_joint | p | q |
|---|---|---|---|---|---|---|---|---|---|---|
| 1 | NSD1 | nuclear receptor binding SET domain protein 1 | 36 | 33 | 36 | 1 | 0.0694 | 0.00748 | 0 | 0.00 |
| 2 | PIK3CA | phosphoinositide-3-kinase, catalytic, alpha polypeptide | 65 | 64 | 24 | 0 | 0.000659 | 0 | 0 | 0.00 |
| 3 | CDKN2A | cyclin-dependent kinase inhibitor 2A | 65 | 65 | 31 | 0 | 0 | 0 | 0 | 0.00 |
| 4 | HRAS | v-Ha-ras Harvey rat sarcoma viral oncogene homolog | 11 | 10 | 6 | 0 | 0.00126 | 0 | 0 | 0.00 |
| 5 | TP53 | tumor protein p53 | 246 | 214 | 153 | 5 | 0 | 0 | 0 | 0.00 |
| 6 | NFE2L2 | nuclear factor (erythroid-derived 2)-like 2 | 18 | 17 | 13 | 0 | 1.00E−06 | 0 | 0 | 0.00 |
| 7 | NOTCH1 | Notch homolog 1, translocation-associated (Drosophila) | 62 | 57 | 62 | 5 | 0.729 | 0.00107 | 1.11E−16 | 0.00 |
| 8 | FAT1 | FAT tumor suppressor homolog 1 (Drosophila) | 80 | 72 | 80 | 2 | 0.0294 | 0.14 | 5.22E−15 | 0.00 |
| 9 | CASP8 | caspase 8, apoptosis-related cysteine peptidase | 27 | 27 | 24 | 0 | 0.0282 | 0.136 | 1.64E−14 | 0.00 |
| 10 | JUB | jub, ajuba homolog (Xenopus laevis) | 19 | 18 | 19 | 1 | 0.383 | 0.275 | 7.54E−14 | 0.00 |
| 11 | MLL2 | myeloid/lymphoid or mixed-lineage leukemia 2 | 58 | 56 | 58 | 3 | 0.242 | 0.519 | 8.58E−14 | 0.00 |
| 12 | FBXW7 | F-box and WD repeat domain containing 7 | 16 | 15 | 14 | 1 | 0.634 | 1.18E−05 | 3.97E−11 | 0.00 |
| 13 | EPHA2 | EPH receptor A2 | 16 | 14 | 15 | 0 | 0.29 | 0.108 | 4.58E−10 | 0.00 |
| 14 | ZNF750 | zinc finger protein 750 | 15 | 13 | 14 | 1 | 0.0158 | 7.38E−05 | 1.01E−09 | 0.00 |
| 15 | FLG | filaggrin | 59 | 48 | 59 | 9 | 0.449 | 0.0488 | 2.18E−09 | 0.00 |
| 16 | B2M | beta-2-microglobulin | 7 | 7 | 6 | 0 | 0.249 | 0.464 | 2.03E−08 | 0.00 |
| 17 | IL32 | interleukin 32 | 4 | 4 | 2 | 0 | 0.915 | 0.000263 | 3.18E−07 | 0.00 |
| 18 | EP300 | E1A binding protein p300 | 25 | 25 | 22 | 1 | 0.15 | 0.00532 | 4.53E−07 | 0.00 |
| 19 | RHOA | ras homolog gene family, member A | 4 | 4 | 1 | 0 | 0.0944 | 6.40E−06 | 2.64E−06 | 0.00 |
| 20 | HLA-A | major histocompatibility complex, class I, A | 9 | 9 | 8 | 2 | 0.176 | 0.22 | 2.80E−06 | 0.00 |
| 21 | CTCF | CCCTC-binding factor (zinc finger protein) | 13 | 11 | 13 | 1 | 0.253 | 0.0674 | 6.15E−06 | 0.01 |
| 22 | RB1 | retinoblastoma 1 (including osteosarcoma) | 10 | 10 | 10 | 2 | 0.155 | 0.493 | 9.84E−06 | 0.01 |
| 23 | TGFBR2 | transforming growth factor, beta receptor II | 11 | 10 | 9 | 1 | 0.591 | 0.54 | 1.40E−05 | 0.01 |
| 24 | CSMD3 | CUB and Sushi multiple domains 3 | 88 | 70 | 87 | 17 | 0.814 | 1 | 1.76E−05 | 0.01 |
| 25 | NECAB1 | N-terminal EF-hand calcium binding protein 1 | 6 | 6 | 6 | 2 | 0.899 | 1 | 1.90E−05 | 0.01 |
| 26 | KRTAP1-5 | keratin associated protein 1-5 | 3 | 3 | 1 | 1 | 0.899 | 0.000775 | 2.07E−05 | 0.01 |
| 27 | MAPK1 | mitogen-activated protein kinase 1 | 4 | 4 | 1 | 0 | 0.231 | 0.000176 | 2.39E−05 | 0.02 |
| 28 | PLSCR1 | phospholipid scramblase 1 | 5 | 5 | 4 | 0 | 0.976 | 0.0101 | 4.32E−05 | 0.03 |
| 29 | CNPY3 | canopy 3 homolog (zebrafish) | 3 | 3 | 1 | 0 | 0.666 | 0.000755 | 6.04E−05 | 0.04 |
| 30 | EPB41L3 | erythrocyte membrane protein band 4.1-like 3 | 16 | 16 | 16 | 5 | 0.96 | 0.0299 | 7.81E−05 | 0.05 |
| 31 | RAC1 | ras-related C3 botulinum toxin substrate 1 (rho family, | 10 | 9 | 8 | 0 | 0.332 | 0.458 | 8.82E−05 | 0.05 |
| | | small GTP binding protein Rac1) | | | | | | | | |
| 32 | CUL3 | cullin 3 | 10 | 10 | 10 | 1 | 0.576 | 0.159 | 0.00013 | 0.07 |
| 33 | TRPV4 | transient receptor potential cation channel, subfamily V | 7 | 7 | 7 | 4 | 0.172 | 0.000541 | 0.00013 | 0.07 |
| 34 | PRB2 | proline-rich protein BstNI subfamily 2 | 11 | 10 | 10 | 3 | 0.943 | 0.0784 | 0.00015 | 0.08 |
| 35 | PRB1 | proline-rich protein BstNI subfamily 1 | 8 | 7 | 7 | 1 | 0.283 | 0.453 | 0.00019 | 0.10 |
| 36 | WHSC1 | Wolf-Hirschhorn syndrome candidate 1 | 11 | 10 | 8 | 0 | 0.00368 | 0.0131 | 0.00026 | 0.13 |
| 37 | STEAP4 | STEAP family member 4 | 10 | 10 | 10 | 1 | 0.95 | 1 | 0.00037 | 0.18 |
| 38 | HIST1H1B | histone cluster 1, H1b | 7 | 7 | 7 | 2 | 0.149 | 0.245 | 0.00038 | 0.18 |
| 39 | KCNA3 | potassium voltage-gated channel, member 3 | 8 | 8 | 8 | 2 | 0.852 | 0.0764 | 0.00039 | 0.18 |
| 40 | EPDR1 | ependymin related protein 1 (zebrafish) | 6 | 6 | 6 | 2 | 0.0509 | 0.00472 | 0.00041 | 0.18 |
| 41 | SLC26A7 | solute carrier family 26, member 7 | 8 | 8 | 8 | 1 | 0.267 | 0.178 | 0.00042 | 0.18 |
| 42 | OR8D4 | olfactory receptor, family 8, subfamily D, member 4 | 6 | 6 | 5 | 0 | 0.967 | 0.192 | 0.00043 | 0.18 |
| 43 | POU4F2 | POU class 4 homeobox 2 | 7 | 7 | 4 | 3 | 0.996 | 0.104 | 0.00044 | 0.19 |
| 44 | FCRL4 | Fc receptor-like 4 | 14 | 13 | 14 | 1 | 0.97 | 0.404 | 0.00045 | 0.19 |
| 45 | TXK | TXK tyrosine kinase | 3 | 3 | 2 | 0 | 0.971 | 0.000725 | 0.00048 | 0.19 |
| 46 | C3orf59 | chromosome 3 open reading frame 59 | 8 | 8 | 4 | 1 | 0.187 | 0.0316 | 0.00056 | 0.22 |
| 47 | RAB32 | RAB32, member RAS oncogene family | 3 | 3 | 3 | 0 | 0.938 | 0.0017 | 0.00060 | 0.23 |
| 48 | KCNT2 | potassium channel, subfamily T, member 2 | 17 | 17 | 16 | 1 | 0.541 | 0.0461 | 0.00075 | 0.28 |
| 49 | MYH9 | myosin, heavy chain 9, non-muscle | 13 | 13 | 13 | 3 | 0.0226 | 0.00669 | 0.00077 | 0.28 |
| 50 | C5orf23 | chromosome 5 open reading frame 23 | 3 | 3 | 3 | 0 | 0.0402 | 0.019 | 0.00087 | 0.31 |

n = number of (nonsilent) mutations in this gene across the individual set;
npat = number of patients (individuals) with at least one nonsilent mutation;
nsite = number of unique sites having a non-silent mutation;
p_cons = p-value for enrichment of mutations at evolutionarily most-conserved sites in gene;
p_joint = p-value for clustering + conservation;
p = p-value (overall);
q = q-value, False Discovery Rate (Benjamini-Hochberg procedure)

TABLE 5
Full list of cancer types with their respective percentage of MYH9 hemizygosity

| | MYH9 | | MYH9 |
|---|---|---|---|
| Human Cancers: | hemizygosity | | hemizygosity |
| HNSCC | 15% | Lung Adenocarcinoma | 40% |
| | | Lung Squamous Cell Carcinoma | 9% |
| Acute Myeloid Leukemia | 1% | Lymphoid Neoplasm Diffuse Large B- | 6% |
| | | cell Lymphoma | |
| Bladder Urothelial Carcinoma | 35% | Ovarian Serous Cystadenocarcinoma | 79% |
| Brain Lower Grade Glioma | 10% | Pancreatic Adenocarcinoma | 15% |
| Breast Invasive Carcinoma | 46% | Prostate Adenocarcinoma | 8% |
| Cervical Squamous Cell | 26% | Sarcoma | 42% |
| Carcinoma and Endocervical | | | |
| Adenocarcinoma | | | |
| Colon and Rectum | 34% | Skin Cutaneous Melanoma | 10% |
| Adenocarcinoma | | | |
| Glioblastoma Multiforme | 38% | Stomach Adenocarcinoma | 29% |
| Kidney Renal Clear Cell | 8% | Thyroid Carcinoma | 17% |
| Carcinoma | | | |
| Kidney Renal Papillary Cell | 26% | Uterine Corpus Endometrial Carcinoma | 11% |
| Carcinoma | | | |
| | Tumor | | Tumor |
| Mouse: | incidence | | incidence |
| heterozygous Myh9 iKO TbRII- | ~26% | homozygous Myh9 iKO TbRII-iKO mice | 100% |
| iKO mice | | | |

While the disclosure has been particularly shown and described with reference to specific embodiments (some of which are preferred embodiments), it should be understood by those having skill in the art that various changes in form and detail may be made therein without departing from the spirit and scope of the present description as set forth herein.

What is claimed is:

1. A method of aiding in diagnosing whether a subject has an aggressive form of a cancer, comprising: testing a sample of a tumor from the subject to determine a mutation in the Myh9 gene or low expression of the Myh9 gene relative to a control, wherein the presence of the mutation and/or the low expression aids in the diagnosis that the individual has an aggressive form of cancer.

2. The method of claim 1, wherein the mutation in the Myh9 gene is selected from the group consisting of A454V, E457K, E465Q, N470S, E530K, T538K, D567N, G696S, L812X, E1131M, S1163X, K1249E, F1261L, A1351P, L1411P, L1485P, and combinations thereof.

3. The method of claim 1, wherein the testing the sample comprises determining a polynucleotide sequence of the Myh9 gene.

4. The method of claim 2, wherein at least one of the mutations are determined

5. The method of claim 1, wherein the cancer is a squamous cell carcinoma of the head and neck or the skin cancer or a breast cancer.

6. The method of claim 5, wherein the squamous cell carcinoma is a head and neck squamous cell carcinoma.

7. A method for identifying an individual as a candidate for treatment with a nuclear export inhibitor comprising testing a sample of a tumor from the subject to determine a mutation in the Myh9 gene and/or low expression of the Myh9 gene relative to a control, wherein the presence of the mutation in the Myh9 and/or the low expression of the Myh9 gene relative to a control indicates that the individual is a candidate for therapy with a nuclear export inhibitor.

8. The method of claim 7, wherein the mutation in the Myh9 gene selected from the group consisting of A454V, E457K, E465Q, N470S, E530K, T538K, D567N, G696S, L812X, E1131M, S1163X, K1249E, F1261L, A1351P, L1411P, L1485P, and combinations thereof.

9. The method of claim 8, further comprising administering to the individual a pharmaceutical composition comprising a nuclear export inhibitor.

10. A method for determining whether tumor cells have defective p53 nuclear transportation comprising testing the tumor cells for a mutation in the Myh9 gene, wherein the presence of the mutation in the Myh9 gene determines that the cells have defective p53 nuclear transportation.

11. The method of claim 10 wherein the mutation in the Myh9 gene is selected from the group consisting of A454V, E457K, E465Q, N470S, E530K, T538K, D567N, G696S, L812X, E1131M, S1163X, K1249E, F1261L, A1351P, L1411P, L1485P, and combinations thereof.

12. The method of claim 10, wherein the determining the defective p53 nuclear transportation further determines that the tumor cells are an aggressive form of tumor cells.

13. The method of claim 10, wherein the tumor cells are a component of a sample of a tumor obtained from an individual diagnosed with cancer.

14. The method of claim 13, wherein the cancer is a squamous cell carcinoma of the head and neck region or the skin or a breast cancer.

15. A method for treating an individual diagnosed with an aggressive cancer, wherein the aggressive cancer comprises cancer cells, which comprise a mutation in the Myh9 gene, comprising administering to the individual a composition comprising an effective amount of a nuclear export inhibitor.
